(19) United States
(12) Patent Application Publication
(10) Pub. No.: US 2026/0191943 A1
(43) Pub. Date: Jul. 9, 2026

(54) Methods for Preparing Conjugated Capsular Saccharide Antigens and Uses Thereof

(71) Applicant: PFIZER INC., New York, NY (US)

(72) Inventors: Caitlyn GALLAGHER, Ringwood, NJ (US); Mingming HAN, Holly Springs, NC (US); James Morgan HARRIS, Chapel Hill, NC (US); Katherine Maurine HORVATH, Durham, NC (US); John Michael JARNAGIN, Pittsboro, NC (US); Isis KANEVSKY, New York, NY (US)

(21) Appl. No.: 19/131,515

(22) PCT Filed: Nov. 17, 2023
(86) PCT No.: PCT/IB2023/061641

§ 371 Date: May 20, 2025

Related U.S. Application Data
(60) Provisional application No. 63/384,523, filed on Nov. 21, 2022.

(51) Int. Cl. A61K 39/09; A61K 39/00; A61K 39/385; A61K 47/64

(52) U.S. Cl. CPC A61K 39/092; A61K 39/385; A61K 47/6415; A61K 47/646; A61K 2039/6037; A61K 2039/605

(57) ABSTRACT

The present invention relates to methods for preparing conjugated capsular saccharide antigens (glycoconjugates), immunogenic compositions comprising said glycoconjugates and uses thereof.

### FIELD OF THE INVENTION

The present invention relates to methods for preparing conjugated capsular saccharide antigens (glycoconjugates), immunogenic compositions comprising said glycoconjugates and uses thereof. Immunogenic compositions of the present invention will typically comprise glycoconjugates, wherein the saccharides are derived from bacterial capsular polysaccharide antigens, in particular a capsular polysaccharide derived from pathogenic bacteria. The invention also relates to vaccination of human subjects, in particular infants and elderly, against infections using said glycoconjugates.

### BACKGROUND OF THE INVENTION

Bacterial cell surface polysaccharides, particularly capsular polysaccharides, have become increasingly important as therapeutic agents. Typically, a cell surface polysaccharide is associated with inducing an immune response in vivo.

Although polysaccharides are immunogenic on their own, conjugation of polysaccharides to protein carriers (glycoconjugate) has been used to improve immunogenicity, particularly in infants and the elderly. Glycoconjugate vaccines are typically obtained by covalent linkage of poorly immunogenic sugar antigens to a protein carrier and play an important role in the prevention of many deadly infectious diseases. In the preparation of conjugate vaccines, selected bacterial strains are grown to supply polysaccharides needed to produce the vaccine. The cells are often grown in fermentors with lysis induced at the end of the fermentation. The lysate broth is then harvested for downstream purification and recovery of the capsular polysaccharide. After conjugation with a carrier protein, the polysaccharide is included in the final vaccine product and confers immunity in the vaccine's target population to the bacteria.

Click conjugation (i.e. conjugation using click chemistry) offers several advantages over the current chemistries used to generate glycoconjugate, such as: increased speed, increased specificity, and lack of organic solvents. However, the azide moiety and alkyne groups which are often used in the click chemistry are absent in almost all naturally existing compounds. Therefore, functionalization of the polysaccharide and the protein carrier are required before conjugation can take place. Introduction of these groups in either the polysaccharide or the carrier protein can be laborious and can introduce structural modification of the polysaccharide which may have an impact on its immunogenicity.

Accordingly, improved methods for functionalization of capsular polysaccharides are needed. In particular methods with reduced number of steps prior to conjugation and which allow for minimal structural modifications of the antigen (i.e. the polysaccharide) are needed.

### SUMMARY OF THE INVENTION

In an aspect, the present invention pertains to a method of making a capsular saccharide glycoconjugate, comprising the steps of:
(a) contacting an isolated Streptococcus pneumoniae capsular saccharide with an azido linker and 4-(4,6-dimethoxy [1,3,5]triazin-2-yl)-4-methyl-morpholinium (DMTMM) to produce an azido incorporated saccharide;
(b) contacting a carrier protein with an agent bearing an N-hydroxysuccinimide (NHS) moiety and an alkyne group to produce an alkyne functionalized carrier protein; and
(c) reacting the azido incorporated saccharide with the alkyne functionalized carrier protein by Cu⁺¹ mediated azide-alkyne cycloaddition reaction, thereby forming the capsular saccharide glycoconjugate.

In an aspect, the Streptococcus pneumoniae is selected from the group consisting of Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.

In an aspect, the azido linker is a compound of formula (I):

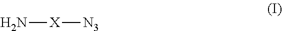

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂;
where n is selected from 1 to 10 and m is selected from 1 to 4.

In an aspect, the azido linker is a compound of formula (II):

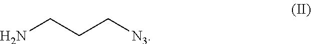

In an aspect, the agent bearing an N-Hydroxysuccinimide (NHS) moiety and an alkyne group is an agent bearing an N-Hydroxysuccinimide (NHS) moiety and a terminal alkyne.

In an aspect, the agent bearing an N-Hydroxysuccinimide (NHS) moiety and an alkyne group is a compound of formula (III):

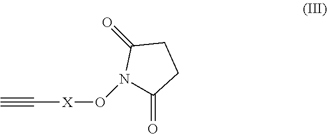

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

In an aspect, the agent bearing an N-Hydroxysuccinimide (NHS) moiety and an alkyne group is a compound of formula (IV):

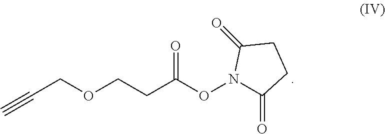 [SMILES: C#CCOCCC(=O)ON1C(=O)CCC1=O]

In an aspect, the carrier protein is selected from the group consisting of CRM197, Streptococcus C5a peptidase (SCP), diphtheria toxoid (DT), tetanus toxoid (TT), meningococcal outer membrane protein (OMP) complex, non-typeable Haemophilus influenzae protein D, and pan HLA DR-binding epitope (PADRE).

In an aspect, the capsular saccharide glycoconjugate has a molecular weight of >1000 kDA.

In an aspect, the capsular saccharide glycoconjugate has a free saccharide % of <30%.

In an aspect, the capsular saccharide glycoconjugate has a conjugate saccharide to protein ratio of 0.5-2.5.

In an aspect, the azido linker is present in an amount that is between 0.01-10 molar equivalent to an amount of polysaccharide Repeat Unit of the azido incorporated saccharide.

In an aspect, the agent bearing an N-Hydroxysuccinimide (NHS) moiety and an alkyne group is present in an amount that is between 0.1-10 molar equivalents to an amount of lysines on the carrier protein.

In an aspect, step (c) is carried out in an aqueous buffer in the presence of copper (I) as catalyst.

In an aspect, the method further comprises purifying the capsular saccharide glycoconjugate after step (c).

In a particular aspect, the invention is directed to a capsular saccharide glycoconjugate produced according to a method comprising the steps of:
(a) contacting an isolated Streptococcus pneumoniae capsular saccharide with an azido linker and 4-(4,6-dimethoxy [1,3,5]triazin-2-yl)-4-methyl-morpholinium (DMTMM) to produce an azido incorporated saccharide;
(b) contacting a carrier protein with an agent bearing an N-hydroxysuccinimide (NHS) moiety and an alkyne group to produce an alkyne functionalized carrier protein; and
(c) reacting the azido incorporated saccharide with the alkyne functionalized carrier protein by Cu⁺¹ mediated azide-alkyne cycloaddition reaction, thereby forming the capsular saccharide glycoconjugate.

In an aspect, the present invention pertains to a method of making a capsular saccharide glycoconjugate, comprising the steps of:
(a) contacting an isolated Streptococcus pneumoniae capsular saccharide with 1-cyano-4-dimethylaminopyridinium tetrafluoroborate (CDAP) to produce a reactive intermediate;
(b) contacting the reactive intermediate with an azido linker to produce an azido incorporated saccharide;
(c) contacting a carrier protein with an agent bearing an N-hydroxysuccinimide (NHS) moiety and an alkyne group to produce an alkyne functionalized carrier protein; and
(d) reacting the azido incorporated saccharide with the alkyne functionalized carrier protein by Cu⁺¹ mediated azide-alkyne cycloaddition reaction, thereby forming the capsular saccharide glycoconjugate.

In an aspect, the Streptococcus pneumoniae is selected from the group consisting of Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.

In an aspect, the azido linker is a compound of formula (I):

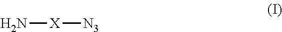

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂;
where n is selected from 1 to 10 and m is selected from 1 to 4.

In an aspect, the azido linker is a compound of formula (II):

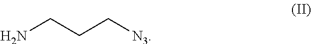

In an aspect, the agent bearing an N-Hydroxysuccinimide (NHS) moiety and an alkyne group is an agent bearing an N-Hydroxysuccinimide (NHS) moiety and a terminal alkyne.

In an aspect, the agent bearing an N-Hydroxysuccinimide (NHS) moiety and an alkyne group is a compound of formula (III):

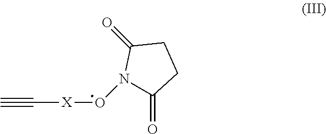

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

In an aspect, the agent bearing an N-Hydroxysuccinimide (NHS) moiety and an alkyne group is a compound of formula (IV):

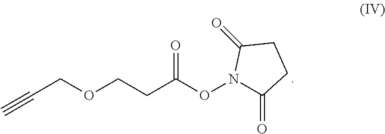 [SMILES: C#CCOCCC(=O)ON1C(=O)CCC1=O]

In an aspect, the carrier protein is selected from the group consisting of CRM197, Streptococcus C5a peptidase (SCP), diphtheria toxoid (DT), tetanus toxoid (TT), meningococcal outer membrane protein (OMP) complex, non-typeable Haemophilus influenzae protein D, and pan HLA DR-binding epitope (PADRE).

In an aspect, the capsular saccharide glycoconjugate has a molecular weight of >1000 kDA.

In an aspect, the capsular saccharide glycoconjugate has a free saccharide % of <30%.

In an aspect, the capsular saccharide glycoconjugate has a conjugate saccharide to protein ratio of 0.5-2.5.

In an aspect, the azido linker is present in an amount that is between 0.01-10 molar equivalent to an amount of polysaccharide Repeat Unit of the azido incorporated saccharide.

In an aspect, the agent bearing an N-Hydroxysuccinimide (NHS) moiety and an alkyne group is present in an amount that is between 0.1-10 molar equivalents to an amount of lysines on the carrier protein.

In an aspect, step (c) is carried out in an aqueous buffer in the presence of copper (I) as catalyst.

In an aspect, the method further comprises purifying the capsular saccharide glycoconjugate after step (c).

In a particular aspect, the invention is directed to a capsular saccharide glycoconjugate produced according to a method comprising the steps of:
(a) contacting an isolated Streptococcus pneumoniae capsular saccharide with 1-cyano-4-dimethylaminopyridinium tetrafluoroborate (CDAP) to produce a reactive intermediate;
(b) contacting the reactive intermediate with an azido linker to produce an azido incorporated saccharide;
(c) contacting a carrier protein with an agent bearing an N-hydroxysuccinimide (NHS) moiety and an alkyne group to produce an alkyne functionalized carrier protein; and
(d) reacting the azido incorporated saccharide with the alkyne functionalized carrier protein by Cu⁺¹ mediated azide-alkyne cycloaddition reaction, thereby forming the capsular saccharide glycoconjugate.

In an aspect, the present invention pertains to a method of making a capsular saccharide glycoconjugate, comprising the steps of:
(a) contacting a primary alcohol of an isolated Streptococcus pneumoniae capsular saccharide with 4-Acetamido-2,2,6,6-tetramethyl-1-oxopiperidinium tetrafluoroborate (TEMPOX) to produce a reactive saccharide intermediate;
(b) contacting the reactive saccharide intermediate with an azido linker to form an imine bond and produce an azido saccharide intermediate;
(c) contacting the azido saccharide intermediate with sodium cyanoborohydride (NaBH3CN) to reduce the imine bond to an amine bond and produce a reduced azido incorporated saccharide;
(d) contacting a carrier protein with an agent bearing an N-hydroxysuccinimide (NHS) moiety and an alkyne group to produce an alkyne functionalized carrier protein; and
(e) reacting the reduced azido incorporated saccharide with the alkyne functionalized carrier protein by Cu⁺¹ mediated azide-alkyne cycloaddition reaction, thereby forming the capsular saccharide glycoconjugate.

In an aspect, the Streptococcus pneumoniae is selected from the group consisting of Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.

In an aspect, the azido linker is a compound of formula (I):

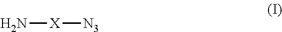

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂;
where n is selected from 1 to 10 and m is selected from 1 to 4.

In an aspect, the azido linker is a compound of formula (II):

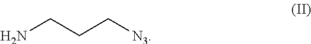

In an aspect, the agent bearing an N-Hydroxysuccinimide (NHS) moiety and an alkyne group is an agent bearing an N-Hydroxysuccinimide (NHS) moiety and a terminal alkyne.

In an aspect, the agent bearing an N-Hydroxysuccinimide (NHS) moiety and an alkyne group is a compound of formula (III):

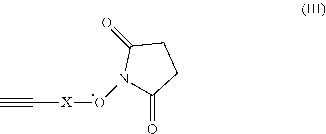

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

In an aspect, the agent bearing an N-Hydroxysuccinimide (NHS) moiety and an alkyne group is a compound of formula (IV):

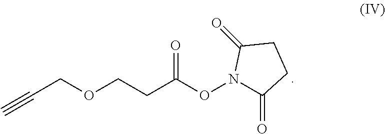 [SMILES: C#CCOCCC(=O)ON1C(=O)CCC1=O]

In an aspect, the carrier protein is selected from the group consisting of CRM197, Streptococcus C5a peptidase (SCP), diphtheria toxoid (DT), tetanus toxoid (TT), meningococcal outer membrane protein (OMP) complex, non-typeable Haemophilus influenzae protein D, and pan HLA DR-binding epitope (PADRE).

In an aspect, the capsular saccharide glycoconjugate has a molecular weight of >1000 kDA.

In an aspect, the capsular saccharide glycoconjugate has a free saccharide % of <30%.

In an aspect, the capsular saccharide glycoconjugate has a conjugate saccharide to protein ratio of 0.5-2.5.

In an aspect, the azido linker is present in an amount that is between 0.01-10 molar equivalent to an amount of polysaccharide Repeat Unit of the azido incorporated saccharide.

In an aspect, the agent bearing an N-Hydroxysuccinimide (NHS) moiety and an alkyne group is present in an amount that is between 0.1-10 molar equivalents to an amount of lysines on the carrier protein.

In an aspect, step (c) is carried out in an aqueous buffer in the presence of copper (I) as catalyst.

In an aspect, the method further comprises purifying the capsular saccharide glycoconjugate after step (c).

In a particular aspect, the invention is directed to a capsular saccharide glycoconjugate produced according to a method comprising the steps of:
(a) contacting a primary alcohol of an isolated Streptococcus pneumoniae capsular saccharide with 4-Acetamido-2,2,6,6-tetramethyl-1-oxopiperidinium tetrafluoroborate (TEMPOX) to produce a reactive saccharide intermediate;
(b) contacting the reactive saccharide intermediate with an azido linker to form an imine bond and produce an azido saccharide intermediate;
(c) contacting the azido saccharide intermediate with sodium cyanoborohydride (NaBH3CN) to reduce the imine bond to an amine bond and produce a reduced azido incorporated saccharide;
(d) contacting a carrier protein with an agent bearing an N-hydroxysuccinimide (NHS) moiety and an alkyne group to produce an alkyne functionalized carrier protein; and
(e) reacting the reduced azido incorporated saccharide with the alkyne functionalized carrier protein by Cu+1 mediated azide-alkyne cycloaddition reaction, thereby forming the capsular saccharide glycoconjugate.

### FIGURES

FIGS. 1A-C show general schemes for the synthetic approaches for the type 3 click conjugation including TEMPOx activation and reductive amination conjugation (RAC) (FIG. 1A), 1-cyano-4-dimethylaminopyridinium tetrafluoroborate (CDAP) hydroxyl group activation and subsequent amide coupling (FIG. 1B), and 4-(4,6-dimethoxy-1,3,5-triazin-2-yl)-4-methyl-morpholinium chloride (DMTMM) carboxylic acid activation and subsequent amide coupling (FIG. 1C).

FIG. 2 shows two proposed routes of CDAP incorporation including NMR analysis (left) and cyanogen bromide classic coupling mechanism (right).

FIG. 3 shows a model for 3-azidopropylamine incorporation into Pn3 polysaccharide based on Buffer:CDAP ratio. Model equation: % azide=19.93-2.902*[Buffer to CDAP ratio; linear fit.

FIG. 4 shows a general scheme for the reaction mechanism of DMTMM coupling of 3-azidopropylamine to Pn3 polysaccharide.

FIG. 5 shows a general scheme for TEMPO/NCS and TEMPOX mediated polysaccharide oxidation.

FIG. 6 shows a model for 3-azidopropylamine incorporation into Pn3 polysaccharide based on Pn3 polysaccharide:DMTMM ratio (g/g).

FIG. 7 shows a general scheme for the preparation of glycoconjugate of the invention prepared using click chemistry and using 3-Azido-1-propylamine as azido linker.

FIG. 8 shows murine immunogenicity (opsonophagocytic titer) results for Pn3-CRM197 glycoconjugates prepared using TEMPOx click and CDAP click chemistries compared to RAC/aqueous traditional chemistry.

FIG. 9 shows murine immunogenicity (opsonophagocytic titer) results for Pn3-CRM197 glycoconjugates prepared using DMTMM click chemistry at two different azide incorporation levels compared to RAC/aqueous traditional chemistry.

### DETAILED DESCRIPTION OF THE INVENTION

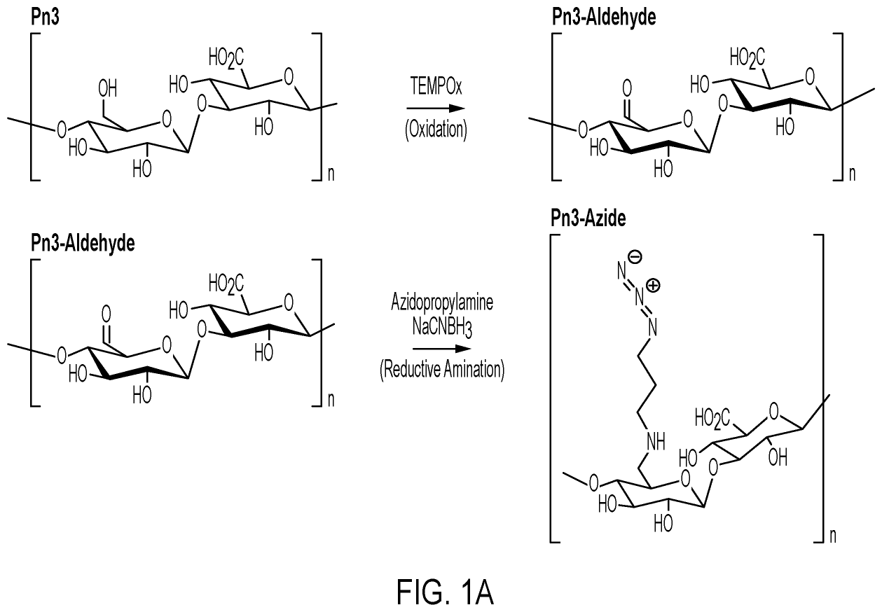

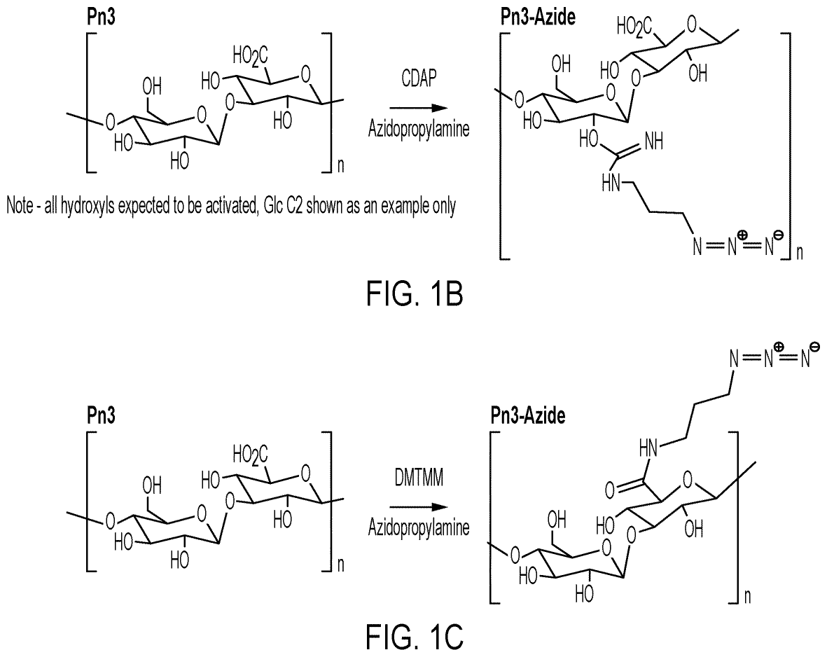

The present inventors have developed new methods for Streptococcus pneumoniae polysaccharide activation via synthetic chemical addition of azido groups and polysaccharide-carrier protein conjugates obtained via these methods. Exemplified herein are synthetic routes for 3-azidopropylamine incorporation in S. pneumoniae serotype 3 (pn3) polysaccharide using TEMPOx, CDAP, and DMTMM chemistries and novel click conjugation processes for Serotype 3-CRM197 (Pn3-CRM), Serotype 3-CRM197 derived peptide, and Serotype 3-PADRE peptide conjugates (FIG. 1). As disclosed herein, models were developed to control the level of azide incorporation and a slow addition CRM197/CuAAC methodology was used to increase the degree of click conjugation and reduce alkyne-alkyne homocoupling. Further, a carrier peptide utilizing a universal T helper epitope such as the pan DR-binding epitope (PADRE) and the MCHII epitopes for CRM197 were click conjugated successfully to the Pn3 azide activated polysaccharide via CDAP and DMTMM chemistries. These methods to generate glycoconjugates have been found to allow for producing glycoconjugates with very low free saccharide, superior immunogenicity, and a good yield.

### 1. Glycoconjugates of the Invention

The present invention is directed in part to conjugated bacterial capsular saccharide antigens (also named glycoconjugates). For the purpose of the invention the term ‘glycoconjugate’ indicates a capsular saccharide (in particular a bacterial capsular saccharide) linked covalently to a carrier protein.

### 1.1 Capsular Saccharide of the Invention

The term “saccharide” throughout this specification may indicate polysaccharide or oligosaccharide and includes both. In an embodiment, saccharide of the invention may be oligosaccharides. Oligosaccharides have a low number of repeat units (typically 5-15 repeat units) and are typically derived synthetically or by hydrolysis of polysaccharides. Preferably though, all of the saccharides of the present invention and in the immunogenic compositions of the present invention are polysaccharides. High molecular weight polysaccharides are able to induce certain antibody immune responses due to the epitopes present on the antigenic surface. The isolation and purification of high molecular weight capsular polysaccharides is preferably contemplated for use in the conjugates, compositions and methods of the present invention. Therefore, in a preferred embodiment of the present invention, the saccharide is a polysaccharide.

Preferably, the saccharide used in the present invention is a bacterial capsular saccharide (also named ‘capsular saccharide’ herein). Capsules are found in several bacteria of medical importance. Bacterial capsules are largely composed of polysaccharides. Capsular saccharides are prepared by standard techniques known to those of ordinary skill in the art.

In a most preferred embodiment of the present invention, the saccharide is a S. pneumoniae capsular polysaccharide.

In an embodiment, the capsular saccharide used in the present invention is a synthetic carbohydrate.

In a preferred embodiment though, the source of bacterial capsular saccharide according to this invention can be bacterial cells. Bacterial strains which can be used as source of capsular saccharide may be obtained from established culture collections (such as for example from the American Type Culture Collection (ATCC, Manassas, VA USA) or the Streptococcal Reference Laboratory (Centers for Disease Control and Prevention, Atlanta, GA USA)) or clinical specimens.

Bacterial capsular saccharides can be obtained directly from bacteria using isolation procedures known to one of ordinary skill in the art (see for example methods disclosed in US2006/0228380, US2006/0228381, US2007/0184071, US2007/0184072, US2007/0231340, and US2008/0102498 and WO2008/118752). They can also be produced using synthetic protocols known to the man skilled in the art.

In case the bacterial capsular saccharide is obtained directly from bacteria, the bacterial cells can be grown in a medium. Following fermentation of bacterial cells that produce the capsular saccharide, the bacterial cells can be lysed to produce a cell lysate. The capsular saccharide may then be isolated from the cell lysate using purification techniques known in the art, including the use of centrifugation, depth filtration, precipitation, ultra-filtration, treatment with activated carbon, diafiltration and/or column chromatography (see, for example, US2006/0228380, US2006/0228381 and WO2008/118752). The purified capsular saccharide can then be used for the preparation of immunogenic conjugates.

The isolated capsular saccharide obtained by purification can be characterized by different parameters including, for example the weight average molecular weight (Mw). The molecular weight of the polysaccharide can be measured by Size Exclusion Chromatography (SEC) combined with Multiangle Laser Light Scattering detector (MALLS).

In an embodiment, the capsular saccharide used in the method of making or part of the glycoconjugate of the present invention is a capsular saccharide from a pathogenic bacteria. Preferably, the capsular saccharide used in the present invention is a capsular saccharide from a pathogenic Streptococcus, a pathogenic Staphylococcus, a pathogenic Enterococcus, a pathogenic Bacillus, a pathogenic Corynebacterium, a pathogenic Listeria, a pathogenic Erysipelothrix, a pathogenic Clostridium, a pathogenic Haemophilus, a pathogenic Neisseria or a pathogenic Escherichia. More preferably, the capsular saccharide used in the present invention is a capsular saccharide from a pathogenic Streptococcus, a pathogenic Neisseria or a pathogenic Escherichia.

In an embodiment, the capsular saccharide used in the present invention is a capsular saccharide from Aeromonas hydrophila and other species (spp.); Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Chlamydia trachomatis, Clostridium botulinum; Clostridium dificile; Clostridium perfringens; Coccidioides immitis; Coccidioides posadasii; Cowdria ruminantium (Heartwater); Coxiella burnetii; Enterococcus faecalis; Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli-enteroinvasive (EIEC); Ehrlichia spp. such as Ehrlichia chajfeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerosporaphilippinensis; Phakopsora pachyrhizi; Plesiomonas shigelloides; Ralstonia solanacearum race 3, biovar 2; Rickettsia prowazekii; Rickettsia rickettsii; Salmonella spp.; Schlerophthora rayssiae var zeae; Shigella spp.; Staphylococcus aureus; Streptococcus; Synchytrium endobioticum; Vibrio cholerae non-01; Vibrio cholerae 01; Vibrio par ahaemo ly ticus and other Vibrios; Vibrio vulnificus; Xanthomonas oryzae; Xylella fastidiosa (citrus variegated chlorosis strain); Yersinia enterocolitica and Yersinia pseudotuberculosis; or Yersinia pestis. Preferably, the capsular saccharide used in the present invention is a capsular saccharide from Enterococcus faecalis, Escherichia coli, Staphylococcus aureus or Streptococcus.

In an embodiment, the capsular saccharide used in the present invention is a capsular saccharide from Haemophilus influenzae, Neisseria meningitidis, S. pneumoniae, S. pyogenes, S. agalactiae, Group C & G Streptococci or Escherichia coli.

More preferably, the capsular saccharide used in the present invention is a capsular saccharide from Neisseria meningitidis, S. pneumoniae, S. agalactiae or Escherichia coli. Even more preferably, the capsular saccharide used in the present invention is a capsular saccharide from S. pneumoniae or S. agalactiae. Even more preferably, the capsular saccharide used in the present invention is a capsular saccharide from S. pneumoniae.

In an embodiment, the capsular saccharide used in the present invention is a capsular saccharide from Staphylococcus aureus. In an embodiment, the capsular saccharide used in the present invention is a capsular saccharide from Staphylococcus aureus type 5 or Staphylococcus aureus type 8.

In an embodiment, the capsular saccharide used in the present invention is a capsular saccharide from Enterococcus faecalis. In yet a further embodiment, the capsular saccharide used in the present invention is a capsular saccharide from Haemophilus influenzae type b.

In a further embodiment, the capsular saccharide used in the present invention is a capsular saccharide from Neisseria meningitidis. In an embodiment the capsular saccharide used in the present invention is a capsular saccharide from N. meningitidis serogroup A (MenA), N. meningitidis serogroup W135 (MenW135), N. meningitidis serogroup Y (MenY), N. meningitidis serogroup X (MenX) or N. meningitidis serogroup C (MenC). In an embodiment the capsular saccharide used in the present invention is a capsular saccharide from N. meningitidis serogroup A (MenA). In an embodiment the capsular saccharide used in the present invention is a capsular saccharide from N. meningitidis serogroup W135 (MenW135). In an embodiment the capsular saccharide used in the present invention is a capsular saccharide from N. meningitidis serogroup Y (MenY). In an embodiment the capsular saccharide used in the present invention is a capsular saccharide from N. meningitidis serogroup C (MenC). In an embodiment the capsular saccharide used in the present invention is a capsular saccharide from N. meningitidis serogroup X (MenX).

In a further embodiment, the capsular saccharide used in the present invention is a capsular saccharide from Escherichia coli. In a further embodiment, the capsular saccharide used in the present invention is a capsular saccharide from Enterococcus faecalis.

In a further embodiment, the capsular saccharide used in the present invention is a capsular saccharide from Streptococcus agalactiae (Group B streptococcus (GBS)). In some embodiments, the capsular saccharide used in the present invention is a capsular saccharide from GBS type Ia, Ib, II, III, IV, V, VI, VII or VIII. In some embodiments, the capsular saccharide used in the present invention is a capsular saccharide from GBS types Ia, Ib, II, III, IV or V.

In a further embodiment, the capsular saccharide used in the present invention is a capsular saccharide from Escherichia coli. In an embodiment, the capsular saccharide used in the present invention is a capsular saccharide from an Escherichia coli part of the Enterovirulent Escherichia coli group (EEC Group) such as Escherichia coli-enterotoxigenic (ETEC), Escherichia coli-enteropathogenic (EPEC), Escherichia coli—O157:H7 enterohemorrhagic (EHEC), or Escherichia coli—enteroinvasive (EIEC). In an embodiment, the capsular saccharide used in the present invention is a capsular saccharide from an Uropathogenic Escherichia coli (UPEC).

In an embodiment, the capsular saccharide used in the present invention is a capsular saccharide from an Escherichia coli serotype selected from the group consisting of serotypes O157:H7, O26:H11, O111:H- and O103:H2. In an embodiment, the capsular saccharide used in the present invention is a capsular saccharide from an Escherichia coli serotype selected from the group consisting of serotypes O6:K2:H1 and O18:K1:H7. In an embodiment, the capsular saccharide used in the present invention is a capsular saccharide from an Escherichia coli serotype selected from the group consisting of serotypes O45:K1, O17:K52:H18, O19:H34 and O7:K1. In an embodiment, the capsular saccharide used in the present invention is a capsular saccharide from an Escherichia coli serotype O104:H4. In an embodiment, the capsular saccharide used in the present invention is a capsular saccharide from an Escherichia coli serotype O1:K12:H7. In an embodiment, the capsular saccharide used in the present invention is a capsular saccharide from an Escherichia coli serotype O127:H6. In an embodiment, the capsular saccharide used in the present invention is a capsular saccharide from an Escherichia coli serotype O139:H28. In an embodiment, the capsular saccharide used in the present invention is a capsular saccharide from an Escherichia coli serotype O128:H2.

In a preferred embodiment, the capsular saccharide used in the present invention is a capsular saccharide from Steptococcus pneumoniae. Preferably, the capsular saccharide used in the present invention is a capsular saccharide from a Streptococcus pneumoniae serotype selected from the group consisting of serotypes 1, 2, 3, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20, 21, 22A, 22F, 23A, 23B, 23F, 24B, 24F, 27, 29, 31, 33B, 33F, 34, 35B, 35F, 38, 72 and 73.

In a preferred embodiment, the capsular saccharide used in the present invention is a capsular saccharide from Streptococcus pneumoniae serotype 3.

In an embodiment, the capsular saccharide used in the present invention is a capsular saccharide from Streptococcus pneumoniae serotype 1. In an embodiment, the capsular saccharide used in the present invention is a capsular saccharide from Streptococcus pneumoniae serotype 2. In an embodiment, the capsular saccharide used in the present invention is a capsular saccharide from Streptococcus pneumoniae serotype 3. In an embodiment, the capsular saccharide used in the present invention is a capsular saccharide from Streptococcus pneumoniae serotype 4. In an embodiment, the capsular saccharide used in the present invention is a capsular saccharide from Streptococcus pneumoniae serotype 5. In an embodiment, the capsular saccharide used in the present invention is a capsular saccharide from Streptococcus pneumoniae serotype 6A. In an embodiment, the capsular saccharide used in the present invention is a capsular saccharide from Streptococcus pneumoniae serotype 6B. In an embodiment, the capsular saccharide used in the present invention is a capsular saccharide from Streptococcus pneumoniae serotype 7C. In an embodiment, the capsular saccharide used in the present invention is a capsular saccharide from Streptococcus pneumoniae serotype 7F. In an embodiment, the capsular saccharide used in the present invention is a capsular saccharide from Streptococcus pneumoniae serotype 8. In an embodiment, the capsular saccharide used in the present invention is a capsular saccharide from Streptococcus pneumoniae serotype 9V. In an embodiment, the capsular saccharide used in the present invention is a capsular saccharide from Streptococcus pneumoniae serotype 9N. In an embodiment, the capsular saccharide used in the present invention is a capsular saccharide from Streptococcus pneumoniae serotype 10A. In an embodiment, the capsular saccharide used in the present invention is a capsular saccharide from Streptococcus pneumoniae serotype 10B. In an embodiment, the capsular saccharide used in the present invention is a capsular saccharide from Streptococcus pneumoniae serotype 11A. In an embodiment, the capsular saccharide used in the present invention is a capsular saccharide from Streptococcus pneumoniae serotype 12F. In an embodiment, the capsular saccharide used in the present invention is a capsular saccharide from Streptococcus pneumoniae serotype 14. In an embodiment, the capsular saccharide used in the present invention is a capsular saccharide from Streptococcus pneumoniae serotype 15A. In an embodiment, the capsular saccharide used in the present invention is a capsular saccharide from Streptococcus pneumoniae serotype 15B. In an embodiment, the capsular saccharide used in the present invention is a capsular saccharide from Streptococcus pneumoniae serotype 15C. In an embodiment, the capsular saccharide used in the present invention is a capsular saccharide from Streptococcus pneumoniae serotype 16F. In an embodiment, the capsular saccharide used in the present invention is a capsular saccharide from Streptococcus pneumoniae serotype 17F. In an embodiment, the capsular saccharide used in the present invention is a capsular saccharide from Streptococcus pneumoniae serotype 18C. In an embodiment, the capsular saccharide used in the present invention is a capsular saccharide from Streptococcus pneumoniae serotype 19A. In an embodiment, the capsular saccharide used in the present invention is a capsular saccharide from Streptococcus pneumoniae serotype 19F. In an embodiment, the capsular saccharide used in the present invention is a capsular saccharide from Streptococcus pneumoniae serotype 20. In an embodiment, the capsular saccharide used in the present invention is a capsular saccharide from Streptococcus pneumoniae serotype 21. In an embodiment, the capsular saccharide used in the present invention is a capsular saccharide from Streptococcus pneumoniae serotype 22A. In an embodiment, the capsular saccharide used in the present invention is a capsular saccharide from Streptococcus pneumoniae serotype 22F. In an embodiment, the capsular saccharide used in the present invention is a capsular saccharide from Streptococcus pneumoniae serotype 23A. In an embodiment, the capsular saccharide used in the present invention is a capsular saccharide from Streptococcus pneumoniae serotype 23B. In an embodiment, the capsular saccharide used in the present invention is a capsular saccharide from Streptococcus pneumoniae serotype 23F. In an embodiment, the capsular saccharide used in the present invention is a capsular saccharide from Streptococcus pneumoniae serotype 24B. In an embodiment, the capsular saccharide used in the present invention is a capsular saccharide from Streptococcus pneumoniae serotype 24F. In an embodiment, the capsular saccharide used in the present invention is a capsular saccharide from Streptococcus pneumoniae serotype 27. In an embodiment, the capsular saccharide used in the present invention is a capsular saccharide from Streptococcus pneumoniae serotype 29. In an embodiment, the capsular saccharide used in the present invention is a capsular saccharide from Streptococcus pneumoniae serotype 31. In an embodiment, the capsular saccharide used in the present invention is a capsular saccharide from Streptococcus pneumoniae serotype 33B. In an embodiment, the capsular saccharide used in the present invention is a capsular saccharide from Streptococcus pneumoniae serotype 33F. In an embodiment, the capsular saccharide used in the present invention is a capsular saccharide from Streptococcus pneumoniae serotype 34. In an embodiment, the capsular saccharide used in the present invention is a capsular saccharide from Streptococcus pneumoniae serotype 35B. In an embodiment, the capsular saccharide used in the present invention is a capsular saccharide from Streptococcus pneumoniae serotype 35F. In an embodiment, the capsular saccharide used in the present invention is a capsular saccharide from Streptococcus pneumoniae serotype 38. In an embodiment, the capsular saccharide used in the present invention is a capsular saccharide from Streptococcus pneumoniae serotype 72. In an embodiment, the capsular saccharide used in the present invention is a capsular saccharide from Streptococcus pneumoniae serotype 73.

In a preferred embodiment, the capsular saccharide used in the present invention (purified before further treatment) has a weight average molecular weight between 50 kDa and 5000 kDa. In a preferred embodiment, the capsular saccharide used in the present invention has a weight average molecular weight between 500 kDa and 5000 kDa. In another preferred embodiment, the capsular saccharide used in the present invention has a weight average molecular weight between 1000 kDa and 5000 kDa.

Any whole number integer within any of the above ranges is contemplated as an embodiment of the disclosure.

Preferably, in order to generate glycoconjugates with advantageous filterability characteristics, immunogenicity and/or yields, sizing of the saccharide to a target molecular weight range is performed prior to the conjugation to a carrier protein.

Advantageously, the size of the purified capsular saccharide is reduced while preserving critical features of the structure of the polysaccharide. Mechanical or chemical sizing maybe employed.

In an embodiment, the size of the purified capsular saccharide is reduced by chemical hydrolysis. Chemical hydrolysis maybe conducted using a mild acid (e.g acetic acid, formic acid, propanoic acid). In an embodiment, chemical hydrolysis is conducted using formic acid. In an embodiment, chemical hydrolysis is conducted using propanoic acid. In a preferred embodiment, chemical hydrolysis is conducted using acetic acid. Chemical hydrolysis may also be conducted using a diluted strong acid (such as diluted hydrochloric acid, diluted sulfuric acid, diluted phosphoric acid, diluted nitric acid or diluted perchloric acid). In an embodiment, chemical hydrolysis is conducted using diluted hydrochloric acid. In an embodiment, chemical hydrolysis is conducted using diluted sulfuric acid. In an embodiment, chemical hydrolysis is conducted using diluted phosphoric acid. In an embodiment, chemical hydrolysis is conducted using diluted nitric acid. In an embodiment, chemical hydrolysis is conducted using diluted perchloric acid.

The size of the purified capsular saccharide can also be reduced by mechanical homogenization. In an embodiment, the size of the purified capsular saccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

The high-pressure homogenization process can be appropriate for reducing the size of the purified capsular saccharide while preserving the structural features of the saccharide.

In a preferred embodiment, the isolated capsular saccharide is sized to a weight average molecular weight between 10 kDa and 1000 kDa. In an embodiment, the isolated capsular saccharide is sized to a weight average molecular weight between 50 kDa and 500 kDa. In an embodiment, the isolated capsular saccharide is sized to a weight average molecular weight between 50 kDa and 400 kDa. In an embodiment, the isolated capsular saccharide is sized to a weight average molecular weight between 50 kDa and 250 kDa.

In an embodiment, the isolated capsular saccharide is sized to a weight average molecular weight between 250 kDa and 1000 kDa. In an embodiment, the isolated capsular saccharide is sized to a weight average molecular weight between 250 kDa and 500 kDa. In an embodiment, the isolated capsular saccharide is sized to a weight average molecular weight between 250 kDa and 400 kDa. In a preferred embodiment, the isolated capsular saccharide is sized to a weight average molecular weight between 200 kDa and 800 kDa.

In an embodiment, the isolated capsular saccharide is sized to a weight average molecular weight of about 250 kDa. In an embodiment, the isolated capsular saccharide is sized to a weight average molecular weight of about 300 kDa. In an embodiment, the isolated capsular saccharide is sized to a weight average molecular weight of about 350 kDa. In an embodiment, the isolated capsular saccharide is sized to a weight average molecular weight of about 400 kDa. In an embodiment, the isolated capsular saccharide is sized to a weight average molecular weight of about 450 kDa. In an embodiment, the isolated capsular saccharide is sized to a weight average molecular weight of about 500 kDa. In an embodiment, the isolated capsular saccharide is sized to a weight average molecular weight of about 550 kDa. In an embodiment, the isolated capsular saccharide is sized to a weight average molecular weight of about 600 kDa. In an embodiment, the isolated capsular saccharide is sized to a weight average molecular weight of about 700 kDa. In an embodiment, the isolated capsular saccharide is sized to a weight average molecular weight of about 800 kDa. In an embodiment, the isolated capsular saccharide is sized to a weight average molecular weight of about 900 kDa. In an embodiment, the isolated capsular saccharide is sized to a weight average molecular weight of about 1000 kDa.

In an embodiment, the isolated capsular saccharide is not sized.

The isolated capsular saccharide described above may be activated (e.g., chemically activated, optionally using any chemical method described for production of bioconjugates e.g. as described in Hermanson, Greg. Bioconjugate Techniques (2013)) to make them capable of reacting (e.g. with an azide-containing linker) and then incorporated into glycoconjugates, as further described herein.

For the purposes of the invention the term ‘glycoconjugate’ indicates a saccharide covalently linked to a carrier protein.

In general, covalent conjugation of saccharides to carriers enhances the immunogenicity of saccharides as it converts them from T-independent antigens to T-dependent antigens, thus allowing priming for immunological memory. Conjugation is particularly useful for pediatric vaccines.

### 1.2 Capsular Saccharide Glycoconjugates of the Invention

In some embodiments, the glycoconjugate of the present invention comprises a capsular saccharide wherein the weight average molecular weight (Mw) of said polysaccharide before conjugation is between 10 kDa and 2,000 kDa.

The weight average molecular weight (Mw) of the saccharide before conjugation refers to the Mw before the activation of the saccharide (i.e. after an eventual sizing step but before reacting the saccharide with an activating agent). In the context of the present invention the Mw of the saccharide is not substantially modified by the activation step and the Mw of the saccharide incorporated in the conjugate is similar to the Mw of the saccharide as measured before activation.

In an embodiment, the glycoconjugate of the present invention comprises a capsular saccharide wherein the weight average molecular weight (Mw) of said saccharide before conjugation is between 50 kDa and 1,000 kDa. In an embodiment, the weight average molecular weight (Mw) is between 50 kDa and 750 kDa. In an embodiment, the weight average molecular weight (Mw) is between 50 kDa and 500 kDa.

In an embodiment, the weight average molecular weight (Mw) is between 50 kDa and 250 kDa. In an embodiment, the weight average molecular weight (Mw) is between 50 kDa and 200 kDa. In an embodiment, the weight average molecular weight (Mw) is between 50 kDa and 150 kDa. In an embodiment, the weight average molecular weight (Mw) is between 50 kDa and 100 kDa.

In an embodiment, the glycoconjugate of the present invention comprises a capsular saccharide wherein the weight average molecular weight (Mw) of said saccharide before conjugation is between 75 kDa and 1,000 kDa. In an embodiment, the weight average molecular weight (Mw) is between 75 kDa and 750 kDa. In an embodiment, the weight average molecular weight (Mw) is between 75 kDa and 500 kDa. In an embodiment, the weight average molecular weight (Mw) is between 75 kDa and 250 kDa. In an embodiment, the weight average molecular weight (Mw) is between 75 kDa and 200 kDa. In an embodiment, the weight average molecular weight (Mw) is between 75 kDa and 150 kDa. In an embodiment, the weight average molecular weight (Mw) is between 75 kDa and 100 kDa.

In an embodiment, the glycoconjugate of the present invention comprises a capsular saccharide wherein the weight average molecular weight (Mw) of said saccharide before conjugation is between 100 kDa and 1,000 kDa. In an embodiment, the weight average molecular weight (Mw) is between 100 kDa and 750 kDa. In an embodiment, the weight average molecular weight (Mw) is between 100 kDa and 500 kDa. In an embodiment, the weight average molecular weight (Mw) is between 100 kDa and 250 kDa. In an embodiment, the weight average molecular weight (Mw) is between 100 kDa and 200 kDa. In an embodiment, the weight average molecular weight (Mw) is between 100 kDa and 150 kDa.

In an embodiment, the glycoconjugate of the present invention comprises a capsular saccharide wherein the weight average molecular weight (Mw) of said saccharide before conjugation is between 150 kDa and 1,000 kDa. In an embodiment, the weight average molecular weight (Mw) is between 150 kDa and 750 kDa. In an embodiment, the weight average molecular weight (Mw) is between 150 kDa and 500 kDa. In an embodiment, the weight average molecular weight (Mw) is between 150 kDa and 250 kDa. In an embodiment, the weight average molecular weight (Mw) is between 150 kDa and 200 kDa.

In an embodiment, the glycoconjugate of the present invention comprises a capsular saccharide wherein the weight average molecular weight (Mw) of said saccharide before conjugation is between 200 kDa and 1,000 kDa. In an embodiment, the weight average molecular weight (Mw) is between 200 kDa and 750 kDa. In an embodiment, the weight average molecular weight (Mw) is between 200 kDa and 500 kDa. In an embodiment, the weight average molecular weight (Mw) is between 200 kDa and 300 kDa. In an embodiment, the weight average molecular weight (Mw) is between 200 kDa and 250 kDa.

In an embodiment, the glycoconjugate of the present invention comprises a capsular saccharide wherein the weight average molecular weight (Mw) of said saccharide before conjugation is between 500 kDa and 1,000 kDa. In an embodiment, the weight average molecular weight (Mw) is between 500 kDa and 750 kDa. In an embodiment, the weight average molecular weight (Mw) is between 500 kDa and 700 kDa. In an embodiment, the weight average molecular weight (Mw) is between 500 kDa and 600 kDa.

Any whole number integer within any of the above ranges is contemplated as an embodiment of the disclosure.

In an embodiment, the glycoconjugate of the present invention comprises a capsular saccharide wherein the weight average molecular weight (Mw) of said saccharide before conjugation is about 1,000 kDa. In an embodiment, the weight average molecular weight (Mw) is about 750 kDa. In an embodiment, the weight average molecular weight (Mw) is about 700 kDa. In an embodiment, the weight average molecular weight (Mw) is about 600 kDa. In an embodiment, the weight average molecular weight (Mw) is about 500 kDa. In an embodiment, the weight average molecular weight (Mw) is about 400 kDa. In an embodiment, the weight average molecular weight (Mw) is about 300 kDa. In an embodiment, the weight average molecular weight (Mw) is about 200 kDa. In an embodiment, the weight average molecular weight (Mw) is about 150 kDa. In an embodiment, the weight average molecular weight (Mw) is about 125 kDa. In an embodiment, the weight average molecular weight (Mw) is about 100 kDa.

In some embodiments, the glycoconjugate of the invention has a weight average molecular weight (Mw) of between 250 kDa and 20,000 kDa. In other embodiments, the glycoconjugate of the invention has a weight average molecular weight (Mw) of between 500 kDa and 15,000 kDa. In yet other embodiments, the glycoconjugate of the invention has a weight average molecular weight (Mw) of between 500 kDa and 10,000 kDa.

In other embodiments, the glycoconjugate of the invention has a weight average molecular weight (Mw) of between 500 kDa and 10,000 kDa. In other embodiments, the glycoconjugate of the invention has a weight average molecular weight (Mw) of between 500 kDa and 7,500 kDa. In still other embodiments, the glycoconjugate of the invention has a weight average molecular weight (Mw) of between 500 kDa and 5,000 kDa. In still other embodiments, the glycoconjugate of the invention has a weight average molecular weight (Mw) of between 500 kDa and 2,500 kDa. In still other embodiments, the glycoconjugate of the invention has a weight average molecular weight (Mw) of between 500 kDa and 2,000 kDa. In still other embodiments, the glycoconjugate of the invention has a weight average molecular weight (Mw) of between 500 kDa and 1,500 kDa. In still other embodiments, the glycoconjugate of the invention has a weight average molecular weight (Mw) of between 500 kDa and 1,000 kDa. In still other embodiments, the glycoconjugate of the invention has a weight average molecular weight (Mw) of between 500 kDa and 750 kDa.

In other embodiments, the glycoconjugate of the invention has a weight average molecular weight (Mw) of between 750 kDa and 10,000 kDa. In other embodiments, the glycoconjugate of the invention has a weight average molecular weight (Mw) of between 750 kDa and 7,500 kDa. In still other embodiments, the glycoconjugate of the invention has a weight average molecular weight (Mw) of between 750 kDa and 5,000 kDa. In still other embodiments, the glycoconjugate of the invention has a weight average molecular weight (Mw) of between 750 kDa and 2,500 kDa. In still other embodiments, the glycoconjugate of the invention has a weight average molecular weight (Mw) of between 750 kDa and 2,000 kDa. In still other embodiments, the glycoconjugate of the invention has a weight average molecular weight (Mw) of between 750 kDa and 1,500 kDa. In still other embodiments, the glycoconjugate of the invention has a weight average molecular weight (Mw) of between 750 kDa and 1,000 kDa.

In other embodiments, the glycoconjugate of the invention has a weight average molecular weight (Mw) of between 1,000 kDa and 10,000 kDa. In other embodiments, the glycoconjugate of the invention has a weight average molecular weight (Mw) of between 1,000 kDa and 7,500 kDa. In still other embodiments, the glycoconjugate of the invention has a weight average molecular weight (Mw) of between 1,000 kDa and 5,000 kDa. In still other embodiments, the glycoconjugate of the invention has a weight average molecular weight (Mw) of between 1,000 kDa and 2,500 kDa. In still other embodiments, the glycoconjugate of the invention has a weight average molecular weight (Mw) of between 1,000 kDa and 2,000 kDa. In still other embodiments, the glycoconjugate of the invention has a weight average molecular weight (Mw) of between 1,000 kDa and 1,500 kDa.

In other embodiments, the glycoconjugate of the invention has a weight average molecular weight (Mw) of between 2,000 kDa and 10,000 kDa. In other embodiments, the glycoconjugate of the invention has a weight average molecular weight (Mw) of between 2,000 kDa and 7,500 kDa. In still other embodiments, the glycoconjugate of the invention has a weight average molecular weight (Mw) of between 2,000 kDa and 5,000 kDa. In still other embodiments, the glycoconjugate of the invention has a weight average molecular weight (Mw) of between 2,000 kDa and 4,000 kDa. In still other embodiments, the glycoconjugate of the invention has a weight average molecular weight (Mw) of between 2,000 kDa and 3,000 kDa. In still other embodiments, the glycoconjugate of the invention has a weight average molecular weight (Mw) of between 2,000 kDa and 3,500 kDa.

In still other embodiments, the glycoconjugate of the invention has a weight average molecular weight (Mw) of between 2,250 kDa and 3,500 kDa.

In preferred embodiment, the glycoconjugate of the invention has a weight average molecular weight (Mw) of between 1,000 kDa and 2,500 kDa.

Any whole number integer within any of the above ranges is contemplated as an embodiment of the disclosure.

In other embodiments, the glycoconjugate of the invention has a weight average molecular weight (Mw) of about 10,000 kDa. In other embodiments, the glycoconjugate of the invention has a weight average molecular weight (Mw) of about 9,000 kDa. In other embodiments, the glycoconjugate of the invention has a weight average molecular weight (Mw) of about 8,000 kDa. In still other embodiments, the glycoconjugate of the invention has a weight average molecular weight (Mw) of about 7,500 kDa. In still other embodiments, the glycoconjugate of the invention has a weight average molecular weight (Mw) of about 6,000 kDa. In still other embodiments, the glycoconjugate of the invention has a weight average molecular weight (Mw) of about 5,000 kDa. In still other embodiments, the glycoconjugate of the invention has a weight average molecular weight (Mw) of about 4,000 kDa. In still other embodiments, the glycoconjugate of the invention has a weight average molecular weight (Mw) of about 3,500 kDa. In still other embodiments, the glycoconjugate of the invention has a weight average molecular weight (Mw) of about 3,250 kDa. In still other embodiments, the glycoconjugate of the invention has a weight average molecular weight (Mw) of about 3,000 kDa. In still other embodiments, the glycoconjugate of the invention has a weight average molecular weight (Mw) of about 2,500 kDa. In still other embodiments, the glycoconjugate of the invention has a weight average molecular weight (Mw) of about 2,000 kDa. In still other embodiments, the glycoconjugate of the invention has a weight average molecular weight (Mw) of about 1,500 kDa. In still other embodiments, the glycoconjugate of the invention has a weight average molecular weight (Mw) of about 1,000 kDa. In still other embodiments, the glycoconjugate of the invention has a weight average molecular weight (Mw) of about 750 kDa.

Another way to characterize the glycoconjugates of the invention is by the number of lysine residues in the carrier protein (e.g., CRM197 or SCP) that become conjugated to the saccharide which can be characterized as a range of conjugated lysines (degree of conjugation). The evidence for lysine modification of the carrier protein, due to covalent linkages to the saccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the glycoconjugate of the invention is between 2 and 15. In an embodiment, the degree of conjugation of the glycoconjugate of the invention is between 2 and 13. In an embodiment, the degree of conjugation of the glycoconjugate of the invention is between 2 and 10. In an embodiment, the degree of conjugation of the glycoconjugate of the invention is between 2 and 8. In an embodiment, the degree of conjugation of the glycoconjugate of the invention is between 2 and 6. In an embodiment, the degree of conjugation of the glycoconjugate of the invention is between 2 and 5. In an embodiment, the degree of conjugation of the glycoconjugate of the invention is between 2 and 4. In an embodiment, the degree of conjugation of the glycoconjugate of the invention is between 3 and 15. In an embodiment, the degree of conjugation of the glycoconjugate of the invention is between 3 and 13. In an embodiment, the degree of conjugation of the glycoconjugate of the invention is between 3 and 10. In an embodiment, the degree of conjugation of the glycoconjugate of the invention is between 3 and 8. In an embodiment, the degree of conjugation of the glycoconjugate of the invention is between 3 and 6. In an embodiment, the degree of conjugation of the glycoconjugate of the invention is between 3 and 5. In an embodiment, the degree of conjugation of the glycoconjugate of the invention is between 3 and 4. In an embodiment, the degree of conjugation of the glycoconjugate of the invention is between 5 and 15. In an embodiment, the degree of conjugation of the glycoconjugate of the invention is between 5 and 10. In an embodiment, the degree of conjugation of the glycoconjugate of the invention is between 8 and 15. In an embodiment, the degree of conjugation of the glycoconjugate of the invention is between 8 and 12. In an embodiment, the degree of conjugation of the glycoconjugate of the invention is between 10 and 15. In an embodiment, the degree of conjugation of the glycoconjugate of the invention is between 10 and 12.

In an embodiment, the degree of conjugation of the glycoconjugate of the invention is about 2. In an embodiment, the degree of conjugation of the glycoconjugate of the invention is about 3. In an embodiment, the degree of conjugation of the glycoconjugate of the invention is about 4. In an embodiment, the degree of conjugation of the glycoconjugate of the invention is about 5. In an embodiment, the degree of conjugation of the glycoconjugate of the invention is about 6. In an embodiment, the degree of conjugation of the glycoconjugate of the invention is about 7. In an embodiment, the degree of conjugation of the glycoconjugate of the invention is about 8. In an embodiment, the degree of conjugation of the glycoconjugate of the invention is about 9. In an embodiment, the degree of conjugation of the glycoconjugate of the invention is about 10, about 11. In an embodiment, the degree of conjugation of the glycoconjugate of the invention is about 12. In an embodiment, the degree of conjugation of the glycoconjugate of the invention is about 13. In an embodiment, the degree of conjugation of the glycoconjugate of the invention is about 14. In an embodiment, the degree of conjugation of the glycoconjugate of the invention is about 15. In a preferred embodiment, the degree of conjugation of the glycoconjugate of the invention is between 4 and 7. In some such embodiments, the carrier protein is CRM197. In other such embodiments, the carrier protein is SCP. In other such emodiments, the carrier protein is diphtheria toxoid (DT). In other such embodiments, the carrier protein is tetanus toxoid (TT). In other such embodiments, the carrier protein is meningococcal outer membrane protein (OMP) complex. In other such embodiments, the carrier protein is non-typeable Haemophilus influenzae protein D. In other such embodiments, the carrier protein is pan HLA DR-binding epitope (PADRE).

The glycoconjugates of the invention may also be characterized by the ratio (weight/weight) of saccharide to carrier protein. In some embodiments, the ratio of saccharide to carrier protein in the glycoconjugate (w/w) is between 0.5 and 3.0. In other embodiments, the saccharide to carrier protein ratio (w/w) is between 0.5 and 2.0. In other embodiments, the saccharide to carrier protein ratio (w/w) is between 0.5 and 1.5. In other embodiments, the saccharide to carrier protein ratio (w/w) is between 0.8 and 1.2. In other embodiments, the saccharide to carrier protein ratio (w/w) is between 0.5 and 1.0. In other embodiments, the saccharide to carrier protein ratio (w/w) is between 1.0 and 1.5. In other embodiments, the saccharide to carrier protein ratio (w/w) is between 1.0 and 2.0. In further embodiments, the saccharide to carrier protein ratio (w/w) is between 0.8 and 1.2. In a preferred embodiment, the ratio of saccharide to carrier protein in the conjugate is between 0.9 and 1.1.

In an embodiment, the saccharide to carrier protein ratio (w/w) is about 0.5. In other embodiments, the saccharide to carrier protein ratio (w/w) is about 0.6. In other embodiments, the saccharide to carrier protein ratio (w/w) is about 0.7. In other embodiments, the saccharide to carrier protein ratio (w/w) is about 0.8. In other embodiments, the saccharide to carrier protein ratio (w/w) is about 0.9. In other embodiments, the saccharide to carrier protein ratio (w/w) is about 1.0. In other embodiments, the saccharide to carrier protein ratio (w/w) is about 1.1. In other embodiments, the saccharide to carrier protein ratio (w/w) is about 1.2. In other embodiments, the saccharide to carrier protein ratio (w/w) is about 1.3. In other embodiments, the saccharide to carrier protein ratio (w/w) is about 1.4. In other embodiments, the saccharide to carrier protein ratio (w/w) is about 1.5. In other embodiments, the saccharide to carrier protein ratio (w/w) is about 1.6. In other embodiments, the saccharide to carrier protein ratio (w/w) is about 1.7. In other embodiments, the saccharide to carrier protein ratio (w/w) is about 1.8. In other embodiments, the saccharide to carrier protein ratio (w/w) is about 1.9. In other embodiments, the saccharide to carrier protein ratio (w/w) is about 2.0. In other embodiments, the saccharide to carrier protein ratio (w/w) is about 2.1. In other embodiments, the saccharide to carrier protein ratio (w/w) is about 2.2. In other embodiments, the saccharide to carrier protein ratio (w/w) is about 2.5. In other embodiments, the saccharide to carrier protein ratio (w/w) is about 2.8. In other embodiments, the saccharide to carrier protein ratio (w/w) is about 3.0. In some such embodiments, the carrier protein is CRM197. In other such embodiments, the carrier protein is SCP.

The glycoconjugates of the invention may also be characterized by the number of covalent linkages between the carrier protein and the saccharide as a function of repeat units of the saccharide. In one embodiment, the glycoconjugate of the invention comprises at least one covalent linkage between the carrier protein and the saccharide for every 4 saccharide repeat units of the saccharide. In another embodiment, the covalent linkage between the carrier protein and the saccharide occurs at least once in every 10 saccharide repeat units of the saccharide. In another embodiment, the covalent linkage between the carrier protein and the saccharide occurs at least once in every 15 saccharide repeat units of the saccharide. In a further embodiment, the covalent linkage between the carrier protein and the saccharide occurs at least once in every 25 saccharide repeat units of the saccharide. In a further embodiment, the covalent linkage between the carrier protein and the saccharide occurs at least once in every 50 saccharide repeat units of the saccharide. In yet a further embodiment, the covalent linkage between the carrier protein and the saccharide occurs at least once in every 100 saccharide repeat units of the saccharide.

In other embodiments, the glycoconjugate of the invention comprises at least one covalent linkage between the carrier protein and the saccharide for every 5 to 10 saccharide repeat units of the saccharide.

In other embodiments, the glycoconjugate of the invention comprises at least one covalent linkage between the carrier protein and the saccharide for every 2 to 7 saccharide repeat units of the saccharide.

In other embodiments, the glycoconjugate of the invention comprises at least one covalent linkage between the carrier protein and the saccharide for every 6 to 11 saccharide repeat units of the saccharide.

In other embodiments, the glycoconjugate of the invention comprises at least one covalent linkage between the carrier protein and the saccharide for every 9 to 14 saccharide repeat units of the saccharide.

In other embodiments, the glycoconjugate of the invention comprises at least one covalent linkage between the carrier protein and the saccharide for every 10 to 20 saccharide repeat units of the saccharide.

In other embodiments, the glycoconjugate of the invention comprises at least one covalent linkage between the carrier protein and the saccharide for every 4 to 25 saccharide repeat units of the saccharide.

In frequent embodiments, the carrier protein is CRM197. In other embodiments, the carrier protein is SCP. In other emodiments, the carrier protein is diphtheria toxoid (DT). In other embodiments, the carrier protein is tetanus toxoid (TT). In other embodiments, the carrier protein is meningococcal outer membrane protein (OMP) complex. In other embodiments, the carrier protein is non-typeable Haemophilus influenzae protein D. In other embodiments, the carrier protein is pan HLA DR-binding epitope (PADRE).

In some embodiments, the carrier protein is CRM197 and the covalent linkage between the CRM197 and the saccharide occurs at least once in every 4, 10, 15 or 25 saccharide repeat units of the saccharide. In other embodiments, the carrier protein is SCP and the covalent linkage between the SCP and the saccharide occurs at least once in every 4, 10, 15 or 25 saccharide repeat units of the saccharide.

The glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In a preferred embodiment, the glycoconjugate comprises less than about 50% of free saccharide compared to the total amount of said saccharide. In a preferred embodiment the glycoconjugate comprises less than about 40% of free saccharide compared to the total amount of said saccharide. In a yet preferred embodiment, the glycoconjugate comprises less than about 25% of free saccharide compared to the total amount of said saccharide. In an even preferred embodiment, the glycoconjugate comprises less than about 20% of free saccharide compared to the total amount of said saccharide. In a yet preferred embodiment, the glycoconjugate comprises less than about 15% of free saccharide compared to the total amount of said saccharide.

The glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules.

Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In a preferred embodiment, at least 30% of the glycoconjugate of the invention has a K_{d} below or equal to 0.3 in a CL-4B column. In a preferred embodiment, at least 40% of the glycoconjugate of the invention has a K_{d} below or equal to 0.3 in a CL-4B column. In a preferred embodiment, at least 45%, 50%, 55%, 60%, 65%, 70%, 75%, 80%, or 85% of the glycoconjugate of the invention has a K_{d} below or equal to 0.3 in a CL-4B column. In a preferred embodiment, at least 60% of the glycoconjugate of the invention has a K_{d} below or equal to 0.3 in a CL-4B column. In a preferred embodiment, between 50% and 80% of the glycoconjugate of the invention has a K_{d} below or equal to 0.3 in a CL-4B column. In a preferred embodiment, between 65% and 80% of the glycoconjugate of the invention has a K_{d} below or equal to 0.3 in a CL-4B column.

### 1.3 Capsular Saccharide Glycoconjugates of the Invention Prepared Using Click Chemistry

The glycoconjugates of the present invention are prepared using click chemistry. The invention also relates to a method of making a glycoconjugate, as disclosed herein.

According to an embodiment of the present invention, the click chemistry comprises three steps, (a) reacting an isolated capsular saccharide with an azido linker and 4-(4,6-dimethoxy [1,3,5]triazin-2-yl)-4-methyl-morpholinium (DMTMM) to produce an azido incorporated saccharide (activation of the saccharide), (b) reacting a carrier protein with an agent bearing an N-Hydroxysuccinimide (NHS) moiety and an alkyne group where the NHS moiety reacts with the amino groups to form an amide linkage thereby obtaining an alkyne functionalized carrier protein (activation of the carrier protein), (c) reacting the azido incorporated saccharide of step (a) with the activated alkyne-carrier protein of step (b) by Cu⁺¹ mediated azide-alkyne cycloaddition reaction to form a capsular saccharide glycoconjugate.

Following step (a) the saccharide is said to be activated and is referred to herein as an “azido incorporated saccharide,” or “activated azido saccharide”.

Following step (b) the carrier is said to be activated and is referred to as “activated carrier”.

As mentioned above, before the activation (a), sizing of the saccharide to a target molecular weight (MW) range can be performed. Therefore, in an embodiment, the isolated saccharide is sized before activation with DMTMM and an azido linker. In an embodiment, the isolated saccharide is sized to any of the target molecular weight (MW) range defined above.

In an embodiment, said azido linker is a compound of formula (I),

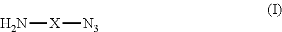

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂;
where n is selected from 1 to 10 and m is selected from 1 to 4.

In an embodiment, said azido linker is a compound of formula (I), wherein X is CH₂(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (I), wherein X is (CH₂CH₂O)ₘCH₂CH₂, wherein m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said azido linker is a compound of formula (I), wherein X is NHCO(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (I), wherein X is NHCO(CH₂CH₂O)ₘCH₂CH₂, where m is selected from 1 to 4. In an embodiment, m is selected from 1 to 3. In an embodiment, m is selected from 1 to 2. In a particular embodiment, m is 1. In another embodiment, m is 2. In yet another embodiment, m is 3.

In yet a further embodiment, m is 4.

In an embodiment, said azido linker is a compound of formula (I), wherein X is OCH₂(CH₂)ₙ, and n is selected from 1 to 10. In an embodiment, n is selected from 1 to 5.

In an embodiment, n is selected from 1 to 4. In an embodiment, n is selected from 1 to 3.

In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (I), wherein X is O(CH₂CH₂O)ₘCH₂CH₂, where m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said azido linker is a compound of formula (II),

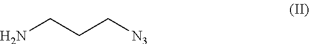

In a preferred embodiment, said azido linker is 3-azido-propylamine.

In an embodiment, said agent bearing an N-Hydroxysuccinimide (NHS) moiety and an alkyne group is an agent bearing an N-Hydroxysuccinimide (NHS) moiety and a terminal alkyne.

In an embodiment, said agent bearing an N-Hydroxysuccinimide (NHS) moiety and an alkyne group is a compound of formula (III),

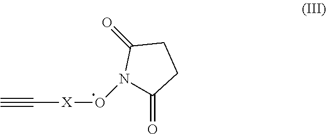

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

In an embodiment, said agent bearing an N-Hydroxysuccinimide (NHS) moiety and an alkyne group is a compound of formula (III), wherein X is CH₂O(CH₂)ₙCH₂C═O, where n is selected from 0 to 10. In an embodiment, n is selected from 0 to 5. In an embodiment, n is selected from 0 to 4. In an embodiment, n is selected from 0 to 3. In an embodiment, n is selected from 0 to 2. In a particular embodiment, n is 0. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said agent bearing an N-Hydroxysuccinimide (NHS) moiety and an alkyne group is a compound of formula (III), wherein X is CH₂O(CH₂CH₂O)ₘ(CH₂)ₙCH₂C═O, where n is selected from 0 to 10 and m is selected from 0 to 4. In an embodiment, n is selected from 0 to 5. In an embodiment, n is selected from 0 to 4. In an embodiment, n is selected from 0 to 3. In an embodiment, n is selected from 0 to 2. In a particular embodiment, n is 0. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10. In an embodiment, m is selected from 0 to 3. In an embodiment, m is selected from 0 to 2. In a particular embodiment, m is 1. In a particular embodiment, m is 1. In another embodiment, m is 2. In yet another embodiment, m is 3. In yet a further embodiment, m is 4.

In an embodiment, n is selected from 0 to 5 and m is selected from 0 to 3. In an embodiment, n is selected from 0 to 5 and m is selected from 0 to 2.

In an embodiment, n is selected from 0 to 4 and m is selected from 0 to 3. In an embodiment, n is selected from 0 to 4 and m is selected from 0 to 2.

In an embodiment, n is selected from 0 to 3 and m is selected from 0 to 3. In an embodiment, n is selected from 0 to 3 and m is selected from 0 to 2.

In an embodiment, n is selected from 0 to 2 and m is selected from 0 to 3. In an embodiment, n is selected from 0 to 2 and m is selected from 0 to 2.

In an embodiment, n is selected from 0 to 1 and m is selected from 0 to 3. In an embodiment, n is selected from 0 to 1 and m is selected from 0 to 2.

In an embodiment, n is 0 and m is 0. In an embodiment, n is 1 and m is 0. In an embodiment, n is 2 and m is 0. In an embodiment, n is 3 and m is 0. In an embodiment, n is 4 and m is 0. In an embodiment, n is 5 and m is 0. In an embodiment, n is 6 and m is 0. In an embodiment, n is 7 and m is 0. In an embodiment, n is 8 and m is 0. In an embodiment, n is 9 and m is 0. In an embodiment, n is 10 and m is 0.

In an embodiment, n is 0 and m is 1. In an embodiment, n is 1 and m is 1. In an embodiment, n is 2 and m is 1. In an embodiment, n is 3 and m is 1. In an embodiment, n is 4 and m is 1. In an embodiment, n is 5 and m is 1. In an embodiment, n is 6 and m is 1. In an embodiment, n is 7 and m is 1. In an embodiment, n is 8 and m is 1. In an embodiment, n is 9 and m is 1. In an embodiment, n is 10 and m is 1.

In an embodiment, n is 0 and m is 2. In an embodiment, n is 1 and m is 2. In an embodiment, n is 2 and m is 2. In an embodiment, n is 3 and m is 2. In an embodiment, n is 4 and m is 2. In an embodiment, n is 5 and m is 2. In an embodiment, n is 6 and m is 2. In an embodiment, n is 7 and m is 2. In an embodiment, n is 8 and m is 2. In an embodiment, n is 9 and m is 2. In an embodiment, n is 10 and m is 2.

In an embodiment, n is 0 and m is 3. In an embodiment, n is 1 and m is 3. In an embodiment, n is 2 and m is 3. In an embodiment, n is 3 and m is 3. In an embodiment, n is 4 and m is 3. In an embodiment, n is 5 and m is 3. In an embodiment, n is 6 and m is 3. In an embodiment, n is 7 and m is 3. In an embodiment, n is 8 and m is 3. In an embodiment, n is 9 and m is 3. In an embodiment, n is 10 and m is 3.

In an embodiment, n is 0 and m is 4. In an embodiment, n is 1 and m is 4. In an embodiment, n is 2 and m is 4. In an embodiment, n is 3 and m is 4. In an embodiment, n is 4 and m is 4. In an embodiment, n is 5 and m is 4. In an embodiment, n is 6 and m is 4. In an embodiment, n is 7 and m is 4. In an embodiment, n is 8 and m is 4. In an embodiment, n is 9 and m is 4. In an embodiment, n is 10 and m is 4.

In an embodiment, said agent bearing an N-Hydroxysuccinimide (NHS) moiety and an alkyne group is a compound of formula (IV):

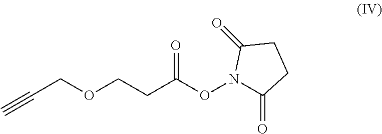 [SMILES: C#CCOCCC(=O)ON1C(=O)CCC1=O]

In an embodiment, step a) comprises contacting the saccharide with DMTMM followed by reacting the saccharide with an azido linker to produce an azido incorporated saccharide. In an embodiment, step a) is carried out in aqueous buffer.

In one embodiment step a) comprises reacting the saccharide with an amount of DMTMM that is between 0.01-10 molar equivalent to the amount of saccharide present in the reaction mixture.

In one embodiment step a) comprises reacting the saccharide with an amount of DMTMM that is between 0.01-5 molar equivalent to the amount of saccharide present in the reaction mixture.

Once the saccharide has been contacted with DMTMM, the activated saccharide is reacted with an azido linker.

In one embodiment step a) further comprises reacting the activated saccharide with an amount of azido linker that is between 0.01-10 molar equivalent to the amount of polysaccharide Repeat Unit of the activated saccharide (molar equivalent of RU).

In one embodiment step a) further comprises reacting the activated saccharide with an amount of azido linker that is between 0.01-8 molar equivalent to the amount of polysaccharide repeat unit of the activated saccharide.

In one embodiment step a) further comprises reacting the activated saccharide with an amount of azido linker that is between 0.01-5 molar equivalent to the amount of polysaccharide repeat unit of the activated saccharide.

In one embodiment step a) further comprises reacting the activated saccharide with an amount of azido linker that is between 0.01-4 molar equivalent to the amount of polysaccharide repeat unit of the activated saccharide.

In one embodiment step a) further comprises reacting the activated saccharide with an amount of azido linker that is between 0.01-3 molar equivalent to the amount of polysaccharide repeat unit of the activated saccharide.

In one embodiment step a) further comprises reacting the activated saccharide with an amount of azido linker that is between 0.01-2 molar equivalent to the amount of polysaccharide repeat unit of the activated saccharide.

In one embodiment step a) further comprises reacting the activated saccharide with an amount of azido linker that is between 0.01-1 molar equivalent to the amount of polysaccharide repeat unit of the activated saccharide.

In one embodiment step a) further comprises reacting the activated saccharide with an amount of azido linker that is between 0.01-0.5 molar equivalent to the amount of polysaccharide repeat unit of the activated saccharide.

In one embodiment step a) further comprises reacting the activated saccharide with an amount of azido linker that is between 0.01-0.1 molar equivalent to the amount of polysaccharide repeat unit of the activated saccharide.

In one embodiment step a) further comprises reacting the activated saccharide with an amount of azido linker that is between 0.05-10 molar equivalent to the amount of polysaccharide repeat unit of the activated saccharide.

In one embodiment step a) further comprises reacting the activated saccharide with an amount of azido linker that is between 0.05-8 molar equivalent to the amount of polysaccharide repeat unit of the activated saccharide.

In one embodiment step a) further comprises reacting the activated saccharide with an amount of azido linker that is between 0.05-5 molar equivalent to the amount of polysaccharide repeat unit of the activated saccharide.

In one embodiment step a) further comprises reacting the activated saccharide with an amount of azido linker that is between 0.05-4 molar equivalent to the amount of polysaccharide repeat unit of the activated saccharide.

In one embodiment step a) further comprises reacting the activated saccharide with an amount of azido linker that is between 0.05-3 molar equivalent to the amount of polysaccharide repeat unit of the activated saccharide.

In one embodiment step a) further comprises reacting the activated saccharide with an amount of azido linker that is between 0.05-2 molar equivalent to the amount of polysaccharide repeat unit of the activated saccharide.

In one embodiment step a) further comprises reacting the activated saccharide with an amount of azido linker that is between 0.05-1 molar equivalent to the amount of polysaccharide repeat unit of the activated saccharide.

In one embodiment step a) further comprises reacting the activated saccharide with an amount of azido linker that is between 0.05-0.5 molar equivalent to the amount of polysaccharide repeat unit of the activated saccharide.

In one embodiment step a) further comprises reacting the activated saccharide with an amount of azido linker that is between 0.05-0.1 molar equivalent to the amount of polysaccharide repeat unit of the activated saccharide.

In one embodiment step a) further comprises reacting the activated saccharide with an amount of azido linker that is between 0.1-10 molar equivalent to the amount of polysaccharide repeat unit of the activated saccharide.

In one embodiment step a) further comprises reacting the activated saccharide with an amount of azido linker that is between 0.1-8 molar equivalent to the amount of polysaccharide repeat unit of the activated saccharide.

In one embodiment step a) further comprises reacting the activated saccharide with an amount of azido linker that is between 0.1-5 molar equivalent to the amount of polysaccharide repeat unit of the activated saccharide.

In one embodiment step a) further comprises reacting the activated saccharide with an amount of azido linker that is between 0.1-4 molar equivalent to the amount of polysaccharide repeat unit of the activated saccharide.

In one embodiment step a) further comprises reacting the activated saccharide with an amount of azido linker that is between 0.1-3 molar equivalent to the amount of polysaccharide repeat unit of the activated saccharide.

In one embodiment step a) further comprises reacting the activated saccharide with an amount of azido linker that is between 0.1-2 molar equivalent to the amount of polysaccharide repeat unit of the activated saccharide.

In one embodiment step a) further comprises reacting the activated saccharide with an amount of azido linker that is between 0.1-1 molar equivalent to the amount of polysaccharide repeat unit of the activated saccharide.

In one embodiment step a) further comprises reacting the activated saccharide with an amount of azido linker that is between 0.1-0.5 molar equivalent to the amount of polysaccharide repeat unit of the activated saccharide.

In one embodiment step a) further comprises reacting the activated saccharide with an amount of azido linker that is between 0.5-10 molar equivalent to the amount of polysaccharide repeat unit of the activated saccharide.

In one embodiment step a) further comprises reacting the activated saccharide with an amount of azido linker that is between 0.5-8 molar equivalent to the amount of polysaccharide repeat unit of the activated saccharide.

In one embodiment step a) further comprises reacting the activated saccharide with an amount of azido linker that is between 0.5-5 molar equivalent to the amount of polysaccharide repeat unit of the activated saccharide.

In one embodiment step a) further comprises reacting the activated saccharide with an amount of azido linker that is between 0.5-4 molar equivalent to the amount of polysaccharide repeat unit of the activated saccharide.

In one embodiment step a) further comprises reacting the activated saccharide with an amount of azido linker that is between 0.5-3 molar equivalent to the amount of polysaccharide repeat unit of the activated saccharide.

In one embodiment step a) further comprises reacting the activated saccharide with an amount of azido linker that is between 0.5-2 molar equivalent to the amount of polysaccharide repeat unit of the activated saccharide.

In one embodiment step a) further comprises reacting the activated saccharide with an amount of azido linker that is between 0.5-1 molar equivalent to the amount of polysaccharide repeat unit of the activated saccharide.

In one embodiment step a) further comprises reacting the activated saccharide with an amount of azido linker that is between 1-10 molar equivalent to the amount of polysaccharide repeat unit of the activated saccharide.

In one embodiment step a) further comprises reacting the activated saccharide with an amount of azido linker that is between 1-8 molar equivalent to the amount of polysaccharide repeat unit of the activated saccharide.

In one embodiment step a) further comprises reacting the activated saccharide with an amount of azido linker that is between 1-5 molar equivalent to the amount of polysaccharide repeat unit of the activated saccharide.

In one embodiment step a) further comprises reacting the activated saccharide with an amount of azido linker that is between 1-4 molar equivalent to the amount of polysaccharide repeat unit of the activated saccharide.

In one embodiment step a) further comprises reacting the activated saccharide with an amount of azido linker that is between 1-3 molar equivalent to the amount of polysaccharide repeat unit of the activated saccharide.

In one embodiment step a) further comprises reacting the activated saccharide with an amount of azido linker that is between 1-2 molar equivalent to the amount of polysaccharide repeat unit of the activated saccharide.

In one embodiment step a) further comprises reacting the activated saccharide with an amount of azido linker that is between 2-10 molar equivalent to the amount of polysaccharide repeat unit of the activated saccharide.

In one embodiment step a) further comprises reacting the activated saccharide with an amount of azido linker that is between 2-8 molar equivalent to the amount of polysaccharide repeat unit of the activated saccharide.

In one embodiment step a) further comprises reacting the activated saccharide with an amount of azido linker that is between 2-5 molar equivalent to the amount of polysaccharide repeat unit of the activated saccharide.

In one embodiment step a) further comprises reacting the activated saccharide with an amount of azido linker that is between 2-4 molar equivalent to the amount of polysaccharide repeat unit of the activated saccharide.

In one embodiment step a) further comprises reacting the activated saccharide with an amount of azido linker that is between 2-3 molar equivalent to the amount of polysaccharide repeat unit of the activated saccharide.

In one embodiment step a) further comprises reacting the activated saccharide with an amount of azido linker that is between 3-10 molar equivalent to the amount of polysaccharide repeat unit of the activated saccharide.

In one embodiment step a) further comprises reacting the activated saccharide with an amount of azido linker that is between 3-8 molar equivalent to the amount of polysaccharide repeat unit of the activated saccharide.

In one embodiment step a) further comprises reacting the activated saccharide with an amount of azido linker that is between 3-5 molar equivalent to the amount of polysaccharide repeat unit of the activated saccharide.

In one embodiment step a) further comprises reacting the activated saccharide with an amount of azido linker that is between 3-4 molar equivalent to the amount of polysaccharide repeat unit of the activated saccharide.

In one embodiment step a) further comprises reacting the activated saccharide with an amount of azido linker that is between 4-10 molar equivalent to the amount of polysaccharide repeat unit of the activated saccharide.

In one embodiment step a) further comprises reacting the activated saccharide with an amount of azido linker that is between 4-8 molar equivalent to the amount of polysaccharide repeat unit of the activated saccharide.

In one embodiment step a) further comprises reacting the activated saccharide with an amount of azido linker that is between 4-5 molar equivalent to the amount of polysaccharide repeat unit of the activated saccharide.

In one embodiment step a) further comprises reacting the activated saccharide with an amount of azido linker that is between 5-10 molar equivalent to the amount of polysaccharide repeat unit of the activated saccharide.

In one embodiment step a) further comprises reacting the activated saccharide with an amount of azido linker that is between 5-8 molar equivalent to the amount of polysaccharide repeat unit of the activated saccharide.

In one embodiment step a) further comprises reacting the activated saccharide with an amount of azido linker that is between 8-10 molar equivalent to the amount of polysaccharide repeat unit of the activated saccharide.

In one embodiment step a) further comprises reacting the activated saccharide with an amount of azido linker that is about 0.01 molar equivalent to the amount of polysaccharide repeat unit of the activated saccharide.

In one embodiment step a) further comprises reacting the activated saccharide with an amount of azido linker that is about 0.05 molar equivalent to the amount of polysaccharide repeat unit of the activated saccharide.

In one embodiment step a) further comprises reacting the activated saccharide with an amount of azido linker that is about 0.1 molar equivalent to the amount of polysaccharide repeat unit of the activated saccharide.

In one embodiment step a) further comprises reacting the activated saccharide with an amount of azido linker that is about 0.5 molar equivalent to the amount of polysaccharide repeat unit of the activated saccharide.

In one embodiment step a) further comprises reacting the activated saccharide with an amount of azido linker that is about 1 molar equivalent to the amount of polysaccharide repeat unit of the activated saccharide.

In one embodiment step a) further comprises reacting the activated saccharide with an amount of azido linker that is about 2 molar equivalents to the amount of polysaccharide repeat unit of the activated saccharide.

In one embodiment step a) further comprises reacting the activated saccharide with an amount of azido linker that is about 3 molar equivalents to the amount of polysaccharide repeat unit of the activated saccharide.

In one embodiment step a) further comprises reacting the activated saccharide with an amount of azido linker that is about 4 molar equivalents to the amount of polysaccharide repeat unit of the activated saccharide.

In one embodiment step a) further comprises reacting the activated saccharide with an amount of azido linker that is about 5 molar equivalents to the amount of polysaccharide repeat unit of the activated saccharide.

In one embodiment step a) further comprises reacting the activated saccharide with an amount of azido linker that is about 8 molar equivalents to the amount of polysaccharide repeat unit of the activated saccharide.

In one embodiment step a) further comprises reacting the activated saccharide with an amount of azido linker that is about 10 molar equivalents to the amount of polysaccharide repeat unit of the activated saccharide.

In one embodiment the degree of activation of the activated saccharide following step a) is between 0.5 to 50%. The degree of activation of the azido saccharide being defined as the percentage of Repeating Unit linked to an azido linker.

In one embodiment the degree of activation of the activated saccharide following step a) is between 1 to 30%. In another embodiment the degree of activation of the activated saccharide following step a) is between 2 to 25%. In another embodiment the degree of activation of the activated saccharide following step a) is between 3 to 20%.

In another embodiment the degree of activation of the activated saccharide following step a) is between 3 to 15%. In another embodiment the degree of activation of the activated saccharide following step a) is between 4 to 15%. In an embodiment the degree of activation of the activated saccharide following step a) is between 1 to 6%.

In an embodiment the degree of activation of the activated saccharide following step a) is between 3 to 6%. In an embodiment the degree of activation of the activated saccharide following step a) is between 10 to 15%.

In an embodiment the degree of activation of the activated saccharide following step a) is about 1%. In an embodiment the degree of activation of the activated saccharide following step a) is about 2%. In an embodiment the degree of activation of the activated saccharide following step a) is about 3%. In an embodiment the degree of activation of the activated saccharide following step a) is about 4%. In an embodiment the degree of activation of the activated saccharide following step a) is about 5%. In an embodiment the degree of activation of the activated saccharide following step a) is about 6%. In an embodiment the degree of activation of the activated saccharide following step a) is about 7%. In an embodiment the degree of activation of the activated saccharide following step a) is about 8%. In an embodiment the degree of activation of the activated saccharide following step a) is about 9%. In an embodiment the degree of activation of the activated saccharide following step a) is about 10%. In an embodiment the degree of activation of the activated saccharide following step a) is about 11%. In an embodiment the degree of activation of the activated saccharide following step a) is about 12%. In an embodiment the degree of activation of the activated saccharide following step a) is about 13%. In an embodiment the degree of activation of the activated saccharide following step a) is about 14%. In an embodiment the degree of activation of the activated saccharide following step a) is about 15%. In an embodiment the degree of activation of the activated saccharide following step a) is about 16%. In an embodiment the degree of activation of the activated saccharide following step a) is about 17%. In an embodiment the degree of activation of the activated saccharide following step a) is about 18%. In an embodiment the degree of activation of the activated saccharide following step a) is about 19%. In an embodiment the degree of activation of the activated saccharide following step a) is about 20%.

In one embodiment step b) comprises reacting the carrier protein with an amount of agent bearing an N-Hydroxysuccinimide (NHS) moiety and an alkyne group that is 0.1-10 molar equivalents to the lysines on the carrier.

In one embodiment step b) comprises reacting the carrier protein with an amount of agent bearing an N-Hydroxysuccinimide (NHS) moiety and an alkyne group that is 0.5-10 molar equivalents to the lysines on the carrier.

In one embodiment step b) comprises reacting the carrier protein with an amount of agent bearing an N-Hydroxysuccinimide (NHS) moiety and an alkyne group that is 1-10 molar equivalents to the lysines on the carrier.

In one embodiment step b) comprises reacting the carrier protein with an amount of agent bearing an N-Hydroxysuccinimide (NHS) moiety and an alkyne group that is 1.5-10 molar equivalents to the lysines on the carrier.

In one embodiment step b) comprises reacting the carrier protein with an amount of agent bearing an N-Hydroxysuccinimide (NHS) moiety and an alkyne group that is 2-10 molar equivalents to the lysines on the carrier.

In one embodiment step b) comprises reacting the carrier protein with an amount of agent bearing an N-Hydroxysuccinimide (NHS) moiety and an alkyne group that is 2.5-10 molar equivalents to the lysines on the carrier.

In one embodiment step b) comprises reacting the carrier protein with an amount of agent bearing an N-Hydroxysuccinimide (NHS) moiety and an alkyne group that is 3-10 molar equivalents to the lysines on the carrier.

In one embodiment step b) comprises reacting the carrier protein with an amount of agent bearing an N-Hydroxysuccinimide (NHS) moiety and an alkyne group that is 5-10 molar equivalents to the lysines on the carrier.

In one embodiment step b) comprises reacting the carrier protein with an amount of agent bearing an N-Hydroxysuccinimide (NHS) moiety and an alkyne group that is 7.5-10 molar equivalents to the lysines on the carrier.

In one embodiment step b) comprises reacting the carrier protein with an amount of agent bearing an N-Hydroxysuccinimide (NHS) moiety and an alkyne group that is 0.1-7.5 molar equivalents to the lysines on the carrier.

In one embodiment step b) comprises reacting the carrier protein with an amount of agent bearing an N-Hydroxysuccinimide (NHS) moiety and an alkyne group that is 0.5-7.5 molar equivalents to the lysines on the carrier.

In one embodiment step b) comprises reacting the carrier protein with an amount of agent bearing an N-Hydroxysuccinimide (NHS) moiety and an alkyne group that is 1-7.5 molar equivalents to the lysines on the carrier.

In one embodiment step b) comprises reacting the carrier protein with an amount of agent bearing an N-Hydroxysuccinimide (NHS) moiety and an alkyne group that is 1.5-7.5 molar equivalents to the lysines on the carrier.

In one embodiment step b) comprises reacting the carrier protein with an amount of agent bearing an N-Hydroxysuccinimide (NHS) moiety and an alkyne group that is 2-7.5 molar equivalents to the lysines on the carrier.

In one embodiment step b) comprises reacting the carrier protein with an amount of agent bearing an N-Hydroxysuccinimide (NHS) moiety and an alkyne group that is 2.5-7.5 molar equivalents to the lysines on the carrier.

In one embodiment step b) comprises reacting the carrier protein with an amount of agent bearing an N-Hydroxysuccinimide (NHS) moiety and an alkyne group that is 3-7.5 molar equivalents to the lysines on the carrier.

In one embodiment step b) comprises reacting the carrier protein with an amount of agent bearing an N-Hydroxysuccinimide (NHS) moiety and an alkyne group that is 5-7.5 molar equivalents to the lysines on the carrier.

In one embodiment step b) comprises reacting the carrier protein with an amount of agent bearing an N-Hydroxysuccinimide (NHS) moiety and an alkyne group that is 0.1-5 molar equivalents to the lysines on the carrier.

In one embodiment step b) comprises reacting the carrier protein with an amount of agent bearing an N-Hydroxysuccinimide (NHS) moiety and an alkyne group that is 0.5-5 molar equivalents to the lysines on the carrier.

In one embodiment step b) comprises reacting the carrier protein with an amount of agent bearing an N-Hydroxysuccinimide (NHS) moiety and an alkyne group that is 1-5 molar equivalents to the lysines on the carrier.

In one embodiment step b) comprises reacting the carrier protein with an amount of agent bearing an N-Hydroxysuccinimide (NHS) moiety and an alkyne group that is 1.5-5 molar equivalents to the lysines on the carrier.

In one embodiment step b) comprises reacting the carrier protein with an amount of agent bearing an N-Hydroxysuccinimide (NHS) moiety and an alkyne group that is 2-5 molar equivalents to the lysines on the carrier.

In one embodiment step b) comprises reacting the carrier protein with an amount of agent bearing an N-Hydroxysuccinimide (NHS) moiety and an alkyne group that is 2.5-5 molar equivalents to the lysines on the carrier.

In one embodiment step b) comprises reacting the carrier protein with an amount of agent bearing an N-Hydroxysuccinimide (NHS) moiety and an alkyne group that is 3-5 molar equivalents to the lysines on the carrier.

In one embodiment step b) comprises reacting the carrier protein with an amount of agent bearing an N-Hydroxysuccinimide (NHS) moiety and an alkyne group that is 0.1-3 molar equivalents to the lysines on the carrier.

In one embodiment step b) comprises reacting the carrier protein with an amount of agent bearing an N-Hydroxysuccinimide (NHS) moiety and an alkyne group that is 0.5-3 molar equivalents to the lysines on the carrier.

In one embodiment step b) comprises reacting the carrier protein with an amount of agent bearing an N-Hydroxysuccinimide (NHS) moiety and an alkyne group that is 1-3 molar equivalents to the lysines on the carrier.

In one embodiment step b) comprises reacting the carrier protein with an amount of agent bearing an N-Hydroxysuccinimide (NHS) moiety and an alkyne group that is 1.5-3 molar equivalents to the lysines on the carrier.

In one embodiment step b) comprises reacting the carrier protein with an amount of agent bearing an N-Hydroxysuccinimide (NHS) moiety and an alkyne group that is 2-3 molar equivalents to the lysines on the carrier.

In one embodiment step b) comprises reacting the carrier protein with an amount of agent bearing an N-Hydroxysuccinimide (NHS) moiety and an alkyne group that is 2.5-3 molar equivalents to the lysines on the carrier.

In one embodiment step b) comprises reacting the carrier protein with an amount of agent bearing an N-Hydroxysuccinimide (NHS) moiety and an alkyne group that is 0.1-2.5 molar equivalents to the lysines on the carrier.

In one embodiment step b) comprises reacting the carrier protein with an amount of agent bearing an N-Hydroxysuccinimide (NHS) moiety and an alkyne group that is 0.5-2.5 molar equivalents to the lysines on the carrier.

In one embodiment step b) comprises reacting the carrier protein with an amount of agent bearing an N-Hydroxysuccinimide (NHS) moiety and an alkyne group that is 1-2.5 molar equivalents to the lysines on the carrier.

In one embodiment step b) comprises reacting the carrier protein with an amount of agent bearing an N-Hydroxysuccinimide (NHS) moiety and an alkyne group that is 1.5-2.5 molar equivalents to the lysines on the carrier.

In one embodiment step b) comprises reacting the carrier protein with an amount of agent bearing an N-Hydroxysuccinimide (NHS) moiety and an alkyne group that is 2-2.5 molar equivalents to the lysines on the carrier.

In one embodiment step b) comprises reacting the carrier protein with an amount of agent bearing an N-Hydroxysuccinimide (NHS) moiety and an alkyne group that is 0.1-2 molar equivalents to the lysines on the carrier.

In one embodiment step b) comprises reacting the carrier protein with an amount of agent bearing an N-Hydroxysuccinimide (NHS) moiety and an alkyne group that is 0.5-2 molar equivalents to the lysines on the carrier.

In one embodiment step b) comprises reacting the carrier protein with an amount of agent bearing an N-Hydroxysuccinimide (NHS) moiety and an alkyne group that is 1-2 molar equivalents to the lysines on the carrier.

In one embodiment step b) comprises reacting the carrier protein with an amount of agent bearing an N-Hydroxysuccinimide (NHS) moiety and an alkyne group that is 1.5-2 molar equivalents to the lysines on the carrier.

In one embodiment step b) comprises reacting the carrier protein with an amount of agent bearing an N-Hydroxysuccinimide (NHS) moiety and an alkyne group that is 0.1-1.5 molar equivalents to the lysines on the carrier.

In one embodiment step b) comprises reacting the carrier protein with an amount of agent bearing an N-Hydroxysuccinimide (NHS) moiety and an alkyne group that is 0.5-1.5 molar equivalents to the lysines on the carrier.

In one embodiment step b) comprises reacting the carrier protein with an amount of agent bearing an N-Hydroxysuccinimide (NHS) moiety and an alkyne group that is 1-1.5 molar equivalents to the lysines on the carrier.

In one embodiment step b) comprises reacting the carrier protein with an amount of agent bearing an N-Hydroxysuccinimide (NHS) moiety and an alkyne group that is 0.1-1 molar equivalents to the lysines on the carrier.

In one embodiment step b) comprises reacting the carrier protein with an amount of agent bearing an N-Hydroxysuccinimide (NHS) moiety and an alkyne group that is 0.5-1 molar equivalents to the lysines on the carrier.

In one embodiment step b) comprises reacting the carrier protein with an amount of agent bearing an N-Hydroxysuccinimide (NHS) moiety and an alkyne group that is 0.1-0.5 molar equivalents to the lysines on the carrier.

In one embodiment step b) comprises reacting the carrier protein with an amount of agent bearing an N-Hydroxysuccinimide (NHS) moiety and an alkyne group that is about 10 molar equivalents to the lysines on the carrier.

In one embodiment step b) comprises reacting the carrier protein with an amount of agent bearing an N-Hydroxysuccinimide (NHS) moiety and an alkyne group that is about 7.5 molar equivalents to the lysines on the carrier.

In one embodiment step b) comprises reacting the carrier protein with an amount of agent bearing an N-Hydroxysuccinimide (NHS) moiety and an alkyne group that is about 5 molar equivalents to the lysines on the carrier.

In one embodiment step b) comprises reacting the carrier protein with an amount of agent bearing an N-Hydroxysuccinimide (NHS) moiety and an alkyne group that is about 3 molar equivalents to the lysines on the carrier.

In one embodiment step b) comprises reacting the carrier protein with an amount of agent bearing an N-Hydroxysuccinimide (NHS) moiety and an alkyne group that is about 2.5 molar equivalents to the lysines on the carrier.

In one embodiment step b) comprises reacting the carrier protein with an amount of agent bearing an N-Hydroxysuccinimide (NHS) moiety and an alkyne group that is about 2 molar equivalents to the lysines on the carrier.

In one embodiment step b) comprises reacting the carrier protein with an amount of agent bearing an N-Hydroxysuccinimide (NHS) moiety and an alkyne group that is about 1.5 molar equivalents to the lysines on the carrier.

In one embodiment step b) comprises reacting the carrier protein with an amount of agent bearing an N-Hydroxysuccinimide (NHS) moiety and an alkyne group that is about 1 molar equivalent to the lysines on the carrier.

In one embodiment step b) comprises reacting the carrier protein with an amount of agent bearing an N-Hydroxysuccinimide (NHS) moiety and an alkyne group that is about 0.5 molar equivalents to the lysines on the carrier.

In one embodiment step b) comprises reacting the carrier protein with an amount of agent bearing an N-Hydroxysuccinimide (NHS) moiety and an alkyne group that is about 0.1 molar equivalents to the lysines on the carrier.

In one embodiment the degree of activation of the activated carrier following step b) is between 1 and 50. The degree of activation of the activated carrier being defined as the number of lysine residues in the carrier protein that become linked to the agent bearing an N-Hydroxysuccinimide (NHS) moiety and an alkyne group.

In an embodiment, the carrier protein is CRM197, which contains 39 lysine residues.

In said embodiment the degree of activation of the activated carrier following step b) may be between 1 to 30. In another embodiment the degree of activation of the activated carrier (CRM197) following step b) is between 5 to 20. In another embodiment the degree of activation of the activated carrier (CRM197) following step b) is between 9 to 18. In another embodiment the degree of activation of the activated carrier (CRM197) following step b) is between 8 to 11. In another embodiment the degree of activation of the activated carrier (CRM197) following step b) is between 15 to 20. In another embodiment the degree of activation of the activated carrier (CRM197) following step b) is about 5. In another embodiment the degree of activation of the activated carrier (CRM197) following step b) is about 6. In another embodiment the degree of activation of the activated carrier (CRM197) following step b) is about 7. In another embodiment the degree of activation of the activated carrier (CRM197) following step b) is about 8. In another embodiment the degree of activation of the activated carrier (CRM197) following step b) is about 9. In another embodiment the degree of activation of the activated carrier (CRM197) following step b) is about 10. In another embodiment the degree of activation of the activated carrier (CRM197) following step b) is about 11. In another embodiment the degree of activation of the activated carrier (CRM197) following step b) is about 12. In another embodiment the degree of activation of the activated carrier (CRM197) following step b) is about 13. In another embodiment the degree of activation of the activated carrier (CRM197) following step b) is about 14. In another embodiment the degree of activation of the activated carrier (CRM197) following step b) is about 15. In another embodiment the degree of activation of the activated carrier (CRM197) following step b) is about 16. In another embodiment the degree of activation of the activated carrier (CRM197) following step b) is about 17. In another embodiment the degree of activation of the activated carrier (CRM197) following step b) is about 18. In another embodiment the degree of activation of the activated carrier (CRM197) following step b) is about 19. In another embodiment the degree of activation of the activated carrier (CRM197) following step b) is about 20. In another embodiment the degree of activation of the activated carrier (CRM197) following step b) is about 21. In another embodiment the degree of activation of the activated carrier (CRM197) following step b) is about 22. In another embodiment the degree of activation of the activated carrier (CRM197) following step b) is about 23. In another embodiment the degree of activation of the activated carrier (CRM197) following step b) is about 24. In another embodiment the degree of activation of the activated carrier (CRM197) following step b) is about 25.

In an embodiment, the carrier protein is SCP or a fragment thereof. In said embodiment the degree of activation of the activated carrier following step b) may be between 1 to 50. In another embodiment the degree of activation of the activated carrier (SCP) following step b) is between 5 to 50. In another embodiment the degree of activation of the activated carrier (SCP) following step b) is between 7 to 45. In another embodiment the degree of activation of the activated carrier (SCP) following step b) is between 5 to 15. In another embodiment the degree of activation of the activated carrier (SCP) following step b) is between 20 to 30. In another embodiment the degree of activation of the activated carrier (SCP) following step b) is between 30 to 50. In another embodiment the degree of activation of the activated carrier (SCP) following step b) is between 30 to 40. In another embodiment the degree of activation of the activated carrier (SCP) following step b) is between 10 to 40. In another embodiment the degree of activation of the activated carrier (SCP) following step b) is about 5. In another embodiment the degree of activation of the activated carrier (SCP) following step b) is about 7. In another embodiment the degree of activation of the activated carrier (SCP) following step b) is about 10. In another embodiment the degree of activation of the activated carrier (SCP) following step b) is about 13. In another embodiment the degree of activation of the activated carrier (SCP) following step b) is about 15. In another embodiment the degree of activation of the activated carrier (SCP) following step b) is about 20. In another embodiment the degree of activation of the activated carrier (SCP) following step b) is about 26. In another embodiment the degree of activation of the activated carrier (SCP) following step b) is about 30. In another embodiment the degree of activation of the activated carrier (SCP) following step b) is about 35. In another embodiment the degree of activation of the activated carrier (SCP) following step b) is about 37. In another embodiment the degree of activation of the activated carrier (SCP) following step b) is about 40. In another embodiment the degree of activation of the activated carrier (SCP) following step b) is about 45. In another embodiment the degree of activation of the activated carrier (SCP) following step b) is about 50.

In an embodiment, the carrier protein is TT or a fragment thereof. In said embodiment the degree of activation of the activated carrier following step b) may be between 1 to 30. In another embodiment the degree of activation of the activated carrier (TT) following step b) is between 5 to 25. In another embodiment the degree of activation of the activated carrier (TT) following step b) is between 7 to 25. In another embodiment the degree of activation of the activated carrier (TT) following step b) is between 10 to 20.

In another embodiment the degree of activation of the activated carrier (TT) following step b) is about 5. In another embodiment the degree of activation of the activated carrier (TT) following step b) is about 7. In another embodiment the degree of activation of the activated carrier (TT) following step b) is about 10. In another embodiment the degree of activation of the activated carrier (TT) following step b) is about 12. In another embodiment the degree of activation of the activated carrier (TT) following step b) is about 15. In another embodiment the degree of activation of the activated carrier (TT) following step b) is about 20. In another embodiment the degree of activation of the activated carrier (TT) following step b) is about 25. In another embodiment the degree of activation of the activated carrier (TT) following step b) is about 30.

In an embodiment, the carrier protein is diphtheria toxoid (DT) or a fragment thereof. In said embodiment the degree of activation of the activated carrier following step b) may be between 1-30 mol Alkyne/mol carrier protein.

In an embodiment, the carrier protein is meningococcal outer membrane protein (OMP) complex or a fragment thereof. In said embodiment the degree of activation of the activated carrier following step b) may be between 1-20 mol Alkyne/mol carrier protein.

In an embodiment, the carrier protein is non-typeable Haemophilus influenzae protein D (PD) or a fragment thereof. In said embodiment the degree of activation of the activated carrier following step b) may be between 1-30 mol Alkyne/mol carrier protein. In an embodiment, the carrier protein is pan HLA DR-binding epitope (PADRE) or a fragment thereof. In said embodiment the degree of activation of the activated carrier following step b) may be between 1-10 mol Alkyne/mol PADRE. In a preferred embodiment the degree of activation of the activated carrier following step b) is about 1 mol alkyne/mol PADRE.

In an embodiment, the conjugation reaction c) is carried out in aqueous buffer. In an embodiment, the conjugation reaction c) is carried out in aqueous buffer in the presence of copper (I) as catalyst. In an embodiment, the conjugation reaction c) is carried out in aqueous buffer in the presence of a reducing agent and copper (II) is added to generate Cu(I) in situ as catalyst. In an embodiment, the conjugation reaction c) is carried out in aqueous buffer in the presence a reducing agent and of copper (I) as catalyst. In a preferred embodiment, the conjugation reaction c) is carried out in aqueous buffer in the presence of copper (I) as catalyst and ascorbate as reducing agent. In an embodiment, THPTA (tris(3-hydroxypropyltriazolylmethyl)amine) and aminoguanidine may be further added to accelerate the click reaction and to protect the protein from side reactions. Therefore, in a preferred embodiment, the conjugation reaction c) is carried out in aqueous buffer in the presence of copper (I) as catalyst and ascorbate as reducing agent, wherein the reaction mixture further comprises THPTA (tris(3-hydroxypropyltriazolylmethyl)amine) and aminoguanidine.

In an embodiment the initial input ratio (weight by weight) of activated azido saccharide to activated alkyne-carrier at step c) is between 0.1 and 3. In an embodiment the initial input ratio (weight by weight) of activated azido saccharide to activated alkyne-carrier at setp c) is between 0.5 and 2. In an embodiment the initial input ratio (weight by weight) of activated azido saccharide to activated alkyne-carrier at step c) is between 0.6 and 1.5. In a preferred embodiment the initial input ratio (weight by weight) of activated azido saccharide to activated alkyne-carrier at setp c) is between 0.8 and 1. In an embodiment the initial input ratio (weight by weight) of activated azido saccharide to activated alkyne-carrier at setp c) is about 0.5. In an embodiment the initial input ratio (weight by weight) of activated azido saccharide to activated alkyne-carrier at setp c) is about 0.6. In an embodiment the initial input ratio (weight by weight) of activated azido saccharide to activated alkyne-carrier at setp c) is about 0.7. In an embodiment the initial input ratio (weight by weight) of activated azido saccharide to activated alkyne-carrier at setp c) is about 0.8. In an embodiment the initial input ratio (weight by weight) of activated azido saccharide to activated alkyne-carrier at setp c) is about 0.9. In an embodiment the initial input ratio (weight by weight) of activated azido saccharide to activated alkyne-carrier at setp c) is about 1. In an embodiment the initial input ratio (weight by weight) of activated azido saccharide to activated alkyne-carrier at setp c) is about 1.1. In an embodiment the initial input ratio (weight by weight) of activated azido saccharide to activated alkyne-carrier at setp c) is about 1.2. In an embodiment the initial input ratio (weight by weight) of activated azido saccharide to activated alkyne-carrier at setp c) is about 1.3. In an embodiment the initial input ratio (weight by weight) of activated azido saccharide to activated alkyne-carrier at setp c) is about 1.4. In an embodiment the initial input ratio (weight by weight) of activated azido saccharide to activated alkyne-carrier at setp c) is about 1.5. In an embodiment the initial input ratio (weight by weight) of activated azido saccharide to activated alkyne-carrier at setp c) is about 1.6. In an embodiment the initial input ratio (weight by weight) of activated azido saccharide to activated alkyne-carrier at setp c) is about 1.7. In an embodiment the initial input ratio (weight by weight) of activated azido saccharide to activated alkyne-carrier at setp c) is about 1.8. In an embodiment the initial input ratio (weight by weight) of activated azido saccharide to activated alkyne-carrier at setp c) is about 1.9. In an embodiment the initial input ratio (weight by weight) of activated azido saccharide to activated alkyne-carrier at setp c) is about 2.

Following the click conjugation reaction, there may remain unreacted azido groups in the conjugates, these may be capped using a suitable azido group capping agent.

Therefore, in an embodiment, following step c), unreacted azido groups in the conjugates, are capped using a suitable azido group capping agent. In one embodiment this azido group capping agent is an agent bearing an alkyne group. In one embodiment this azido group capping agent is an agent bearing a terminal alkyne. In one embodiment this azido group capping agent is an agent bearing a cycloalkyne.

In an embodiment, said azido group capping agent is a compound of formula (V),

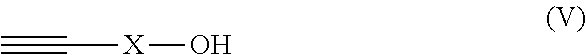

wherein X is (CH₂)ₙ wherein n is selected from 1 to 15.

In one embodiment this azido group capping agent is propargyl alcohol.

Therefore, in an embodiment, following step (c) the process further comprises a step of capping the unreacted azido groups remained in the conjugates with an azido group capping agent.

In an embodiment the capping of the unreacted azido groups is performed with an amount of capping agent that is between 0.05 to 20 molar equivalents to the amount of polysaccharide repeat unit of the activated saccharide.

In an embodiment the capping of the unreacted azido groups is performed with an amount of capping agent that is between 0.1 to 15 molar equivalents to the amount of polysaccharide repeat unit of the activated saccharide.

In an embodiment the capping of the unreacted azido groups is performed with an amount of capping agent that is between 0.5 to 10 molar equivalents to the amount of polysaccharide repeat unit of the activated saccharide.

In an embodiment the capping of the unreacted azido groups is performed with an amount of capping agent that is between 0.5 to 5 molar equivalents to the amount of polysaccharide repeat unit of the activated saccharide.

In an embodiment the capping of the unreacted azido groups is performed with an amount of capping agent that is between 0.5 to 2 molar equivalents to the amount of polysaccharide repeat unit of the activated saccharide.

In an embodiment the capping of the unreacted azido groups is performed with an amount of capping agent that is between 0.5 to 1 molar equivalents to the amount of polysaccharide repeat unit of the activated saccharide.

In an embodiment the capping of the unreacted azido groups is performed with an amount of capping agent that is between 1 to 2 molar equivalents to the amount of polysaccharide repeat unit of the activated saccharide.

In an embodiment the capping of the unreacted azido groups is performed with an amount of capping agent that is between 0.75 to 1.5 molar equivalents to the amount of polysaccharide repeat unit of the activated saccharide.

In an embodiment the capping of the unreacted azido groups is performed with an amount of capping agent that is about 1 molar equivalent to the amount of polysaccharide repeat unit of the activated saccharide.

In an embodiment the capping of the unreacted azido groups is performed with an amount of capping agent that is about 1.5 molar equivalent to the amount of polysaccharide repeat unit of the activated saccharide.

In an embodiment the capping of the unreacted azido groups is performed with an amount of capping agent that is about 0.5 molar equivalent to the amount of polysaccharide repeat unit of the activated saccharide.

In an embodiment the capping of the unreacted azido groups is performed with an amount of capping agent that is about 2 molar equivalents to the amount of polysaccharide repeat unit of the activated saccharide.

Following the click conjugation reaction, unreacted alkyne groups may remain present in the conjugates, these may be capped using a suitable alkyne group capping agent. In one embodiment this alkyne group capping agent is an agent bearing an azido group.

In an embodiment, said alkyne group capping agent is a compound of formula (VI),

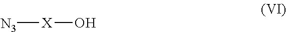

wherein X is (CH₂)ₙ wherein n is selected from 1 to 15.

In one embodiment this alkyne group capping agent is 3-azido-1-propanol.

Therefore, in an embodiment, following step (c) the process further comprises a step of capping the unreacted alkyne groups remained in the conjugates with an alkyne group capping agent.

In an embodiment the capping of the unreacted alkyne groups is performed with an amount of capping agent that is between 0.05 to 20 molar equivalents to the amount of polysaccharide repeat unit of the activated saccharide.

In an embodiment the capping of the unreacted alkyne groups is performed with an amount of capping agent that is between 0.1 to 15 molar equivalents to the amount of polysaccharide repeat unit of the activated saccharide.

In an embodiment the capping of the unreacted alkyne groups is performed with an amount of capping agent that is between 0.5 to 10 molar equivalents to the amount of polysaccharide repeat unit of the activated saccharide.

In an embodiment the capping of the unreacted alkyne groups is performed with an amount of capping agent that is between 0.5 to 5 molar equivalents to the amount of polysaccharide repeat unit of the activated saccharide.

In an embodiment the capping of the unreacted alkyne groups is performed with an amount of capping agent that is between 0.5 to 2 molar equivalents to the amount of polysaccharide repeat unit of the activated saccharide.

In an embodiment the capping of the unreacted alkyne groups is performed with an amount of capping agent that is between 0.5 to 1 molar equivalents to the amount of polysaccharide repeat unit of the activated saccharide.

In an embodiment the capping of the unreacted alkyne groups is performed with an amount of capping agent that is between 1 to 5 molar equivalents to the amount of polysaccharide repeat unit of the activated saccharide.

In an embodiment the capping of the unreacted alkyne groups is performed with an amount of capping agent that is between 1 to 2 molar equivalents to the amount of polysaccharide repeat unit of the activated saccharide.

In an embodiment the capping of the unreacted alkyne groups is performed with an amount of capping agent that is between 1.5 to 2.5 molar equivalents to the amount of polysaccharide repeat unit of the activated saccharide.

In an embodiment the capping of the unreacted alkyne groups is performed with an amount of capping agent that about 0.5 molar equivalent to the amount of polysaccharide repeat unit of the activated saccharide.

In an embodiment the capping of the unreacted alkyne groups is performed with an amount of capping agent that about 1 molar equivalent to the amount of polysaccharide repeat unit of the activated saccharide.

In an embodiment the capping of the unreacted alkyne groups is performed with an amount of capping agent that about 1.5 molar equivalent to the amount of polysaccharide repeat unit of the activated saccharide.

In an embodiment the capping of the unreacted alkyne groups is performed with an amount of capping agent that about 2 molar equivalents to the amount of polysaccharide repeat unit of the activated saccharide.

In an embodiment the capping of the unreacted alkyne groups is performed with an amount of capping agent that about 2.5 molar equivalents to the amount of polysaccharide repeat unit of the activated saccharide.

In an embodiment the capping of the unreacted alkyne groups is performed with an amount of capping agent that about 5 molar equivalents to the amount of polysaccharide repeat unit of the activated saccharide.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (ion exchange chromatography, DEAE or hydrophobic interaction chromatography), and depth filtration. Therefore, in one embodiment the process for producing the glycoconjugate of the present invention comprises the step of purifying the glycoconjugate after it is produced.

In an aspect, the invention provides a glycoconjugate produced according to any of the methods disclosed herein.

### 1.5 Carrier Protein of the Glycoconjugates of the Invention

A component of the glycoconjugate is a carrier protein to which the saccharide is conjugated. The terms “protein carrier” or “carrier protein” or “carrier” may be used interchangeably herein. The terms “protein carrier” or “carrier protein” or “carrier” include proteins and peptides (such as the exemplified PADRE peptide) Carrier proteins should be amenable to standard conjugation procedures.

In a preferred embodiment, the carrier protein of the glycoconjugate of the invention is selected in the group consisting of: DT (Diphtheria toxoid), TT (tetanus toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP). Another suitable carrier protein is pan HLA DR-binding epitope (PADRE).

In a preferred embodiment, the carrier protein of the glycoconjugate of the invention is TT, DT, DT mutants (such as CRM₁₉₇), a C5a peptidase from Streptococcus (SCP), meningococcal outer membrane protein complex (OMPC), non-typeable Haemophilus influenzae protein D (PD), and pan HLA DR-binding epitope (PADRE).

In an embodiment, the carrier protein of the glycoconjugate of the invention is DT (Diphtheria toxoid). In another embodiment, the carrier protein of the glycoconjugate of the invention is TT (tetanus toxoid).

In another embodiment, the carrier protein of the glycoconjugate of the invention is PD (H. influenzae protein D; see, e.g., EP0594610 B).

In a preferred embodiment, the carrier protein of the glycoconjugate of the invention is CRM₁₉₇ or a C5a peptidase from Streptococcus (SCP).

In a preferred embodiment, the carrier protein of the glycoconjugate of the invention is CRM₁₉₇. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the carrier protein of the glycoconjugate of the invention is the A chain of CRM₁₉₇ (see CN103495161). In an embodiment, the carrier protein of the glycoconjugate of the invention is the A chain of CRM₁₉₇ obtained via expression by genetically recombinant E. coli (see CN103495161).

In other preferred embodiments, the carrier protein of the glycoconjugate of the invention is SCP (Streptococcal C5a Peptidase). Two important species of P-hemolytic streptococci, Streptococcus pyogenes (group A Streptococcus, GAS) and Streptococcus agalactiae (group B Streptococcus, GBS), which cause a variety of serious human infections that range from mild cases of pharyngitis and impetigo to serious invasive diseases such as necrotizing fasciitis (GAS) and neonatal sepsis (GBS) have developed a way to defeat this immune response. All human isolates of P-hemolytic streptococci, including GAS and GBS, produce a highly conserved cell-wall protein SCP (Streptococcal C5a Peptidase) that specifically inactivates C5a. The scp genes from GAS and GBS encode a polypeptide containing between 1,134 and 1,181 amino acids (Brown et al., PNAS, 2005, vol. 102, no. 51 pages 18391-18396). The first 31 residues are the export signal presequence and are removed upon passing through the cytoplasmic membrane.

The next 68 residues serve as a pro-sequence and must be removed to produce active SCP. The next 10 residues can be removed without loss of protease activity. At the other end, starting with Lys-1034, are four consecutive 17-residue motifs followed by a cell sorting and cell-wall attachment signal. This combined signal is composed of a 20-residue hydrophilic sequence containing an LPTTND sequence, a 17-residue hydrophobic sequence, and a short basic carboxyl terminus.

SCP can be divided in domains (see FIG. 1B of Brown et al., PNAS, 2005, vol. 102, no. 51 pages 18391-18396). These domains are the Pre/Pro domain (which comprises the export signal presequence (commonly the first 31 residues) and the pro-sequence (commonly the next 68 residues)), the protease domain (which is splitted in two part (protease part 1 commonly residues 89-333/334 and protease domain part 2 and commonly residues 467/468-583/584), the protease-associated domain (PA domain) (commonly residues 333/334-467/468), three fibronectin type III (Fn) domains (Fn1, commonly residues 583/584-712/713; Fn2, commonly residues 712/713-928/929/930; commonly Fn3, residues 929/930-1029/1030/1031) and a cell wall anchor domain (commonly redisues 1029/1030/1031 to the C-terminus).

In an embodiment, the carrier protein of the glycoconjugate of the invention is an SCP from GBS (SCPB). An example of SCPB is provided at SEQ ID.NO: 3 of WO97/26008. See also SEQ ID NO: 3 of WO00/34487.

In another preferred embodiments, the carrier protein of the glycoconjugate of the invention is an SCP from GAS (SCPA). Examples of SCPA can be found at SEQ ID.No.1 and SEQ ID.No.2 of WO97/26008. See also SEQ ID NO: 1, 2 and 23 of WO00/34487.

In a preferred embodiment, the carrier protein of the glycoconjugate of the invention is an enzymatically inactive SCP.

In other preferred embodiments, the carrier protein of the glycoconjugate of the invention is an enzymatically inactive SCP from GBS (SCPB).

In another preferred embodiments, the carrier protein of the glycoconjugate of the invention is an enzymatically inactive SCP from GAS (SCPA).

In an embodiment, the carrier protein of the glycoconjugate of the invention is a fragment of an SCP. In an embodiment, the carrier protein of the glycoconjugate of the invention is a fragment of an SCPA. Preferably, the carrier protein of the glycoconjugate of the invention is a fragment of an SCPB.

In an embodiment, the carrier protein of the glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the glycoconjugate of the invention is an enzymatically inactive fragment of an SCP which comprises the protease domain, the protease-associated domain (PA domain) and two of the three fibronectin type III (Fn) domains but does not comprise the export signal presequence, the pro-sequence and the cell wall anchor domain.

In an embodiment, the carrier protein of the glycoconjugate of the invention is an enzymatically inactive fragment of an SCP. In an embodiment, said enzymatically inactive fragment of SCP comprises the protease domain, the protease-associated domain (PA domain) and the three fibronectin type III (Fn) domains but does not comprise the export signal presequence, the pro-sequence and the cell wall anchor domain.

In an embodiment, the carrier protein of the glycoconjugate of the invention is an enzymatically inactive fragment of an SCPA. In an embodiment, said enzymatically inactive fragment of an SCPA comprises the protease domain, the protease-associated domain (PA domain) and the three fibronectin type III (Fn) domains but does not comprise the export signal presequence, the pro-sequence and the cell wall anchor domain.

In a preferred embodiment, the carrier protein of the glycoconjugate of the invention is an enzymatically inactive fragment of SCPB. Preferably, said enzymatically inactive fragment of SCPB comprises the protease domain, the protease-associated domain (PA domain) and the three fibronectin type III (Fn) domains but does not comprise the export signal presequence, the pro-sequence and the cell wall anchor domain. In an embodiment, the enzymatic activity of SCP is inactivated by replacing at least one amino acid of the wild type sequence. In an embodiment, said replacement is selected from the group consisting of D130A, H193A, N295A and S512A. The numbers indicate the amino acid residue position in the peptidase according to the numbering of SEQ ID NO: 1 of WO00/34487.

Therefore, in an embodiment, the carrier protein of the glycoconjugate of the invention is an enzymatically inactive SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive SCPA where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A.

In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive SCPB where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A.

In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of an SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the enzymatic activity of SCP is inactivated by replacing at least two amino acids of the wild type sequence. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In an embodiment, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

Therefore, in an embodiment, the carrier protein of the glycoconjugate of the invention is an enzymatically inactive SCP where said inactivation is accomplished by replacing at least two amino acids of the wild type sequence. Preferably, said replacement of at least two amino acids is in the protease domain. In an embodiment, said replacement of at least two amino acid is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive SCPA where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive SCPB where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of an SCP where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least one amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the enzymatic activity of SCP is inactivated by replacing at least three amino acids of the wild type sequence. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

Therefore, in an embodiment, the carrier protein of the glycoconjugate of the invention is an enzymatically inactive SCP where said inactivation is accomplished by replacing at least three amino acids of the wild type sequence. Preferably, said replacement of at least three amino acids is in the protease domain. In an embodiment, said replacement of at least three amino acid is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive SCPA where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive SCPB where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of an SCP where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the enzymatic activity of SCP is inactivated by replacing at least four amino acids of the wild type sequence. In an embodiment, said at least four amino acids replacements are D130A, H193A, N295A and S512A.

Therefore, in an embodiment, the carrier protein of the glycoconjugate of the invention is an enzymatically inactive SCP where said inactivation is accomplished by replacing at least four amino acids of the wild type sequence. Preferably, said replacement of at least four amino acids is in the protease domain. In an embodiment, said replacement of at least four amino acid is in part 1 of the protease domain. In an embodiment, said replacement of at least four amino acid is in part 2 of the protease domain. In an embodiment, said at least four amino acids replacements are D130A, H193A, N295A and S512A In an embodiment, the carrier protein of the glycoconjugate of the invention is an enzymatically inactive SCPA where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A In an embodiment, the carrier protein of the glycoconjugate of the invention is an enzymatically inactive SCPB where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A In an embodiment, the carrier protein of the glycoconjugate of the invention is an enzymatically inactive fragment of an SCP where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A In an embodiment, the carrier protein of the glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A In an embodiment, the carrier protein of the glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least one amino acids is in part 2 of the protease domain. In an embodiment, said at least four amino acids replacements are D130A, H193A, N295A and S512A In an embodiment, the carrier protein of the glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acids is in part 2 of the protease domain. In an embodiment, said at least four amino acids replacements are D130A, H193A, N295A and S512A In a particular embodiment, the carrier protein of the glycoconjugate of the invention is an enzymatically inactive fragment of SCP which consists of SEQ ID NO: 41.

In a particular embodiment, the carrier protein of the glycoconjugate of the invention is an enzymatically inactive fragment of SCP which consists of SEQ ID NO: 42.

SEQ ID NO: 41:
MAKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKVVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYVVEDMAGNITYTPVTKLLEGHSNKPEQ
SEQ ID NO: 41 is 950 amino acids long.
SEQ ID NO: 42:
AKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKVVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYVVEDMAGNITYTPVTKLLEGHSNKPEQ
SEQ ID NO: 42 is 949 amino acids long.

In a particular embodiment, the carrier protein of the glycoconjugate of the invention is an enzymatically inactive fragment of SCP consisting of a polypeptide having at least 90% identity with SEQ ID NO: 41.

In a particular embodiment, the carrier protein of the glycoconjugate of the invention is an enzymatically inactive fragment of SCP consisting of a polypeptide having at least 95% identity with SEQ ID NO: 41.

In a particular embodiment, the carrier protein of the glycoconjugate of the invention is an enzymatically inactive fragment of SCP consisting of a polypeptide having at least 99% identity with SEQ ID NO: 41.

In a particular embodiment, the carrier protein of the glycoconjugate of the invention is an enzymatically inactive fragment of SCP consisting of a polypeptide having at least 99.5% identity with SEQ ID NO: 41.

In a particular embodiment, the carrier protein of the glycoconjugate of the invention is an enzymatically inactive fragment of SCP consisting of a polypeptide having at least 99.8% identity with SEQ ID NO: 41.

In a particular embodiment, the carrier protein of the glycoconjugate of the invention is an enzymatically inactive fragment of SCP consisting of a polypeptide having at least 99.85% identity with SEQ ID NO: 41.

In a particular embodiment, the carrier protein of the glycoconjugate of the invention is an enzymatically inactive fragment of SCP consisting of a polypeptide having at least 90% identity with SEQ ID NO: 42.

In a particular embodiment, the carrier protein of the glycoconjugate of the invention is an enzymatically inactive fragment of SCP consisting of a polypeptide having at least 95% identity with SEQ ID NO: 42.

In a particular embodiment, the carrier protein of the glycoconjugate of the invention is an enzymatically inactive fragment of SCP consisting of a polypeptide having at least 99% identity with SEQ ID NO: 42.

In a particular embodiment, the carrier protein of the glycoconjugate of the invention is an enzymatically inactive fragment of SCP consisting of a polypeptide having at least 99.5% identity with SEQ ID NO: 42.

In a particular embodiment, the carrier protein of the glycoconjugate of the invention is an enzymatically inactive fragment of SCP consisting of a polypeptide having at least 99.8% identity with SEQ ID NO: 42.

In a particular embodiment, the carrier protein of the glycoconjugate of the invention is an enzymatically inactive fragment of SCP consisting of a polypeptide having at least 99.85% identity with SEQ ID NO: 42.

### 2 Immunogenic Compositions

### 2.1 Combinations of Glycoconjugates of the Invention

In an embodiment the invention relates to an immunogenic composition comprising a glycoconjugate of the invention (as disclosed at section 1 above).

In an embodiment the invention relates to an immunogenic composition comprising a glycoconjugate of the invention and comprising from 1 to 25 different glycoconjugates.

In an embodiment the invention relates to an immunogenic composition comprising a glycoconjugate of the invention and comprising from 1 to 25 glycoconjugates from different serotypes of S. pneumoniae (1 to 25 pneumococcal conjugates). In one embodiment the invention relates to an immunogenic composition comprising glycoconjugates from 7, 8, 9, 10, 11, 12, 13, 14, 15, 16, 17, 18, 19, 20, 21, 22, 23, 24 or 25 different serotypes of S. pneumoniae. In one embodiment the immunogenic composition comprises glycoconjugates from 16 or 20 different serotypes of S. pneumoniae. In an embodiment the immunogenic composition is a 7, 8, 9, 10, 11, 12, 13, 14, 15, 16, 17, 18, 19 or 20-valent pneumococcal conjugate compositions. In an embodiment the immunogenic composition is a 14, 15, 16, 17, 18 or 19-valent pneumococcal conjugate compositions. In an embodiment the immunogenic composition is a 16-valent pneumococcal conjugate composition. In an embodiment the immunogenic composition is a 19-valent pneumococcal conjugate composition. In an embodiment the immunogenic composition is a 20-valent pneumococcal conjugate composition.

In an embodiment the immunogenic composition is a 21, 22, 23, 24 or 25-valent pneumococcal conjugate compositions. In an embodiment the immunogenic composition is a 21-valent pneumococcal conjugate composition. In an embodiment the immunogenic composition is a 22-valent pneumococcal conjugate composition. In an embodiment the immunogenic composition is a 23-valent pneumococcal conjugate composition. In an embodiment the immunogenic composition is a 24-valent pneumococcal conjugate composition. In an embodiment the immunogenic composition is a 25-valent pneumococcal conjugate composition.

In an embodiment the invention relates to an immunogenic composition comprising a glycoconjugate of the invention and comprising glycoconjugates from S. pneumoniae serotypes 1, 3, 4, 5, 6B, 7F, 9V, 14, 18C, 19F and 23F. In an embodiment the immunogenic composition is an 11-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 9V, 14, 18C, 19A, 19F and 23F. In an embodiment the immunogenic composition is a 13-valent pneumococcal conjugate compositions.

In an embodiment the invention relates to an immunogenic composition comprising a glycoconjugate of the invention and further comprising glycoconjugates from S. pneumoniae serotypes 1, 3, 4, 5, 6A, 6B, 7F, 9V, 14, 18C, 19A, 19F, 22F, 23F and 33F.

In an embodiment the immunogenic composition is a 15-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F. In an embodiment the immunogenic composition is a 20-valent pneumococcal conjugate compositions.

In an embodiment the invention relates to an immunogenic composition comprising a glycoconjugate of the invention and further comprising glycoconjugates from S. pneumoniae serotypes 1, 2, 3, 4, 5, 6A, 6B, 7F, 8, 9V, 10A, 11A, 12F, 14, 15B, 18C, 19A, 19F, 22F, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 9N, 10A, 11A, 12F, 14, 15B, 18C, 19A, 19F, 22F, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 2, 4, 5, 6A, 6B, 7F, 8, 9V, 9N, 10A, 11A, 12F, 14, 15B, 18C, 19A, 19F, 22F, 23F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 2, 4, 5, 6A, 6B, 7F, 8, 9V, 9N, 10A, 11A, 12F, 14, 15B, 17F, 18C, 19A, 19F, 22F, 23F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 2, 3, 4, 5, 6A, 6B, 7F, 8, 9V, 9N, 10A, 11A, 12F, 14, 15B, 17F, 18C, 19A, 19F, 20, 22F, 23F and 33F. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F and 33F. In an embodiment the immunogenic composition is a 22-valent pneumococcal conjugate compositions.

In an embodiment the invention relates to an immunogenic composition comprising a glycoconjugate of the invention and further comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 24F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 33F and 35B. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 24F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 33F and 35B. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 24F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 33F and 35B. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F, 33F and 35B. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 24F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 24F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 24F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 24F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 24F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 24F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F and 33F. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 33F and 35B. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 24F, 33F and 35B. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 24F, 33F and 35B. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In an embodiment the immunogenic composition is a 25-valent pneumococcal conjugate compositions.

In an embodiment the invention relates to an immunogenic composition comprising a glycoconjugate of the invention selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment the invention relates to an immunogenic composition comprising a glycoconjugate of the invention selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 21-valent pneumococcal conjugate compositions.

In an embodiment the invention relates to an immunogenic composition comprising a glycoconjugate of the invention and comprising twenty one glycoconjugates selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 22-valent pneumococcal conjugate compositions.

In an embodiment the invention relates to an immunogenic composition comprising a glycoconjugate of the invention and comprising at least one glycoconjugate selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38.

In an embodiment the invention relates to an immunogenic composition comprising a glycoconjugate of the invention and comprising twenty two glycoconjugates selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a glycoconjugate of the invention and comprising twenty three glycoconjugates selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 2, 9N, 15A, 17F, 20, 23A, 23B, 24F and 35B. In an embodiment the immunogenic composition is a 10-valent pneumococcal conjugate compositions.

In an embodiment the invention relates to an immunogenic composition comprising a glycoconjugate of the invention and comprising glycoconjugates from S. pneumoniae serotypes 2, 9N, 15A, 17F, 19A, 19F, 20, 23A, 23B, 24F and 35B. In an embodiment the immunogenic composition is a 12-valent pneumococcal conjugate compositions.

In an embodiment the invention relates to an immunogenic composition comprising a glycoconjugate of the invention and comprising glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a glycoconjugate of the invention and omprising glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 25-valent pneumococcal conjugate compositions.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate of the invention is conjugated to CRM₁₉₇. In an embodiment of any of the above immunogenic compositions, the glycoconjugate of the invention is conjugated to SCP. In an embodiment of any of the above immunogenic compositions, the glycoconjugate of the invention is conjugated to DT. In an embodiment of any of the above immunogenic compositions, the glycoconjugate of the invention is conjugated to TT. In an embodiment of any of the above immunogenic compositions, the glycoconjugate of the invention is conjugated to OMPC. In an embodiment of any of the above immunogenic compositions, the glycoconjugate of the invention is conjugated to PD. In an embodiment of any of the above immunogenic compositions, the glycoconjugate of the invention is conjugated to PADRE.

In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM₁₉₇. In an embodiment of any of the above immunogenic compositions, the glycoconjugate from S. pneumoniae serotype 12F is conjugated to CRM₁₉₇. In an embodiment of any of the above immunogenic compositions, the glycoconjugate from S. pneumoniae serotype 10A is conjugated to CRM₁₉₇. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇.

In an embodiment of any of the above immunogenic compositions, the glycoconjugates of any of the above immunogenic compositions are all individually conjugated to CRM₁₉₇.

In an embodiment of any of the above immunogenic compositions, the glycoconjugate of the invention is conjugated to SCP and the other glycoconjugate(s) is/are all individually conjugated to CRM₁₉₇.

In an embodiment, the glycoconjugates from S. pneumoniae serotypes 1, 4, 5, 6B, 7F, 9V, 14 and/or 23F of any of the above immunogenic compositions are individually conjugated to PD.

In an embodiment, the glycoconjugate from S. pneumoniae serotype 18C of any of the above immunogenic compositions is conjugated to TT.

In an embodiment, the glycoconjugate from S. pneumoniae serotype 19F of any of the above immunogenic compositions is conjugated to DT.

In an embodiment, the glycoconjugates from S. pneumoniae serotypes 1, 4, 5, 6B, 7F, 9V, 14 and/or 23F of any of the above immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment the above immunogenic compositions comprise from 8 to 25 different serotypes of S. pneumoniae.

Compositions of the invention may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

### 2.2 Dosage of the Immunogenic Compositions of the Invention

The amount of glycoconjugate(s) in each dose is selected as an amount which induces an immunoprotective response without significant, adverse side effects in typical vaccinees. Such amount will vary depending upon which specific immunogen is employed and how it is presented.

The amount of a particular glycoconjugate in an immunogenic composition can be calculated based on total saccharide for that conjugate (conjugated and non-conjugated).

For example, a glycoconjugate with 20% free saccharide will have about 80 μg of conjugated saccharide and about 20 μg of nonconjugated saccharide in a 100 μg saccharide dose. The amount of glycoconjugate can vary depending upon the bacteria and bacteria serotype. The saccharide concentration can be determined by the uronic acid assay.

The “immunogenic amount” of the different saccharide components in the immunogenic composition, may diverge and each may comprise about 0.5 μg, about 0.75 μg, about 1 μg, about 2 μg, about 3 μg, about 4 μg, about 5 μg, about 6 μg, about 7 μg, about 8 μg, about 9 μg, about 10 μg, about 15 μg, about 20 μg, about 30 μg, about 40 μg, about 50 μg, about 60 μg, about 70 μg, about 80 μg, about 90 μg, or about 100 μg of any particular saccharide antigen.

Generally, each dose will comprise 0.1 μg to 100 μg of saccharide. In an embodiment each dose will comprise 0.1 μg to 100 μg of saccharide. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of serotype 3 polysaccharide. Any whole number integer within any of the above ranges is contemplated as an embodiment of the disclosure.

In an embodiment, each dose will comprise about 0.5 μg of saccharide. In an embodiment, each dose will comprise about 0.55 μg of saccharide. In an embodiment, each dose will comprise about 0.75 μg of saccharide. In an embodiment, each dose will comprise about 1.0 μg of saccharide. In an embodiment, each dose will comprise about 1.1 μg of saccharide. In an embodiment, each dose will comprise about 1.5 μg of saccharide. In an embodiment, each dose will comprise about 2.0 μg of saccharide. In an embodiment, each dose will comprise about 2.2 μg of saccharide. In an embodiment, each dose will comprise about 2.5 μg of saccharide. In an embodiment, each dose will comprise about 3.0 μg of saccharide. In an embodiment, each dose will comprise about 3.5 μg of saccharide. In an embodiment, each dose will comprise about 4.0 μg of saccharide. In an embodiment, each dose will comprise about 4.4 μg of saccharide. In an embodiment, each dose will comprise about 5.0 μg of saccharide. In an embodiment, each dose will comprise about 5.5 μg of saccharide. In an embodiment, each dose will comprise about 6.0 μg of saccharide.

Generally, each dose will comprise 0.1 μg to 100 μg of saccharide for a given bacteria or serotype. In an embodiment each dose will comprise 0.1 μg to 100 μg of saccharide for a given bacteria or serotype. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of accharide for a given bacteria or serotype. Any whole number integer within any of the above ranges is contemplated as an embodiment of the disclosure.

In an embodiment, each dose will comprise about 0.5 μg of saccharide for each particular glycoconjugate. In an embodiment, each dose will comprise about 0.55 μg of saccharide for each particular glycoconjugate. In an embodiment, each dose will comprise about 0.75 μg of saccharide for each particular glycoconjugate. In an embodiment, each dose will comprise about 1.0 μg of saccharide for each particular glycoconjugate. In an embodiment, each dose will comprise about 1.1 μg of saccharide for each particular glycoconjugate. In an embodiment, each dose will comprise about 1.5 μg of saccharide for each particular glycoconjugate. In an embodiment, each dose will comprise about 2.0 μg of saccharide for each particular glycoconjugate. In an embodiment, each dose will comprise about 2.2 μg of saccharide for each particular glycoconjugate. In an embodiment, each dose will comprise about 2.5 μg of saccharide for each particular glycoconjugate. In an embodiment, each dose will comprise about 3.0 μg of saccharide for each particular glycoconjugate. In an embodiment, each dose will comprise about 3.5 μg of saccharide for each particular glycoconjugate. In an embodiment, each dose will comprise about 4.0 μg of saccharide for each particular glycoconjugate. In an embodiment, each dose will comprise about 4.4 μg of saccharide for each particular glycoconjugate. In an embodiment, each dose will comprise about 5.0 μg of saccharide for each particular glycoconjugate. In an embodiment, each dose will comprise about 5.5 μg of saccharide for each particular glycoconjugate. In an embodiment, each dose will comprise about 6.0 μg of saccharide for each particular glycoconjugate.

2.3 Carrier Amount Generally, each dose will comprise 10 μg to 150 μg of carrier protein, particularly 15 μg to 100 μg of carrier protein, more particularly 25 μg to 75 μg of carrier protein, and even more particularly 40 μg to 60 μg of carrier protein. In an embodiment, said carrier protein is CRM₁₉₇. In an embodiment, said carrier protein is SCP. In an embodiment, said carrier protein is DT. In an embodiment, said carrier protein is TT. In an embodiment, said carrier protein is OMPC. In an embodiment, said carrier protein is PD. In an embodiment, said carrier protein is PADRE.

In an embodiment, each dose will comprise about 10 μg, about 15 μg, about 20 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg or about 75 μg of carrier protein.

In an embodiment, each dose will comprise about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg or about 75 μg of carrier protein.

In an embodiment, each dose will comprise about 30 μg of carrier protein. In an embodiment, each dose will comprise about 31 μg of carrier protein. In an embodiment, each dose will comprise about 32 μg of carrier protein. In an embodiment, each dose will comprise about 33 μg of carrier protein. In an embodiment, each dose will comprise about 34 μg of carrier protein. In an embodiment, each dose will comprise about 45 μg of carrier protein.

In an embodiment, each dose will comprise about 40 μg of carrier protein. In an embodiment, each dose will comprise about 41 μg of carrier protein. In an embodiment, each dose will comprise about 42 μg of carrier protein. In an embodiment, each dose will comprise about 43 μg of carrier protein. In an embodiment, each dose will comprise about 44 μg of carrier protein. In an embodiment, each dose will comprise about 45 μg of carrier protein.

In an embodiment, each dose will comprise about 48 μg of carrier protein. In an embodiment, each dose will comprise about 49 μg of carrier protein. In an embodiment, each dose will comprise about 50 μg of carrier protein. In an embodiment, each dose will comprise about 51 μg of carrier protein. In an embodiment, each dose will comprise about 52 μg of carrier protein. In an embodiment, each dose will comprise about 53 μg of carrier protein.

In an embodiment, said carrier protein is CRM₁₉₇.

In an embodiment, said carrier protein is SCP.

In an embodiment, said carrier protein is DT.

In an embodiment, said carrier protein is TT.

In an embodiment, said carrier protein is OMPC.

In an embodiment, said carrier protein is PD.

In an embodiment, said carrier protein is PADRE.

### 2.4 Further Antigens

Immunogenic compositions of the invention comprise conjugated saccharide antigen(s) (glycoconjugate(s)). They may also further include antigen(s) from other pathogen(s), particularly from bacteria and/or viruses. Preferred further antigens are selected from: a diphtheria toxoid (D), a tetanus toxoid (T), a pertussis antigen (P), which is typically acellular (Pa), a hepatitis B virus (HBV) surface antigen (HBsAg), a hepatitis A virus (HAV) antigen, a conjugated Haemophilus influenzae type b capsular saccharide (Hib), inactivated poliovirus vaccine (IPV).

In an embodiment, the immunogenic compositions of the invention comprise D-T-Pa. In an embodiment, the immunogenic compositions of the invention comprise D-T-Pa-Hib, D-T-Pa-IPV or D-T-Pa-HBsAg. In an embodiment, the immunogenic compositions of the invention comprise D-T-Pa-HBsAg-IPV or D-T-Pa-HBsAg-Hib. In an embodiment, the immunogenic compositions of the invention comprise D-T-Pa-HBsAg-IPV-Hib.

Pertussis antigens: Bordetella pertussis causes whooping cough. Pertussis antigens in vaccines are either cellular (whole cell, in the form of inactivated B. pertussis cells) or acellular. Preparation of cellular pertussis antigens is well documented (e.g., it may be obtained by heat inactivation of phase I culture of B. pertussis). Preferably, however, the invention uses acellular antigens. Where acellular antigens are used, it is preferred to use one, two or (preferably) three of the following antigens: (1) detoxified pertussis toxin (pertussis toxoid, or PT); (2) filamentous hemagglutinin (FHA); (3) pertactin (also known as the 69 kilodalton outer membrane protein). FHA and pertactin may be treated with formaldehyde prior to use according to the invention. PT is preferably detoxified by treatment with formaldehyde and/or glutaraldehyde. Acellular pertussis antigens are preferably adsorbed onto one or more aluminum salt adjuvants. As an alternative, they may be added in an unadsorbed state. Where pertactin is added then it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is most preferred. Inactivated poliovirus vaccine: Poliovirus causes poliomyelitis. Rather than use oral poliovirus vaccine, preferred embodiments of the invention use IPV. Prior to administration to patients, polioviruses must be inactivated, and this can be achieved by treatment with formaldehyde. Poliomyelitis can be caused by one of three types of poliovirus. The three types are similar and cause identical symptoms, but they are antigenically different and infection by one type does not protect against infection by others. It is therefore preferred to use three poliovirus antigens in the invention: poliovirus Type 1 (e.g., Mahoney strain), poliovirus Type 2 (e.g., MEF-1 strain), and poliovirus Type 3 (e.g., Saukett strain). The viruses are preferably grown, purified and inactivated individually, and are then combined to give a bulk trivalent mixture for use with the invention.

Diphtheria toxoid: Corynebacterium diphtheriae causes diphtheria. Diphtheria toxin can be treated (e.g., using formalin or formaldehyde) to remove toxicity while retaining the ability to induce specific anti-toxin antibodies after injection. These diphtheria toxoids are used in diphtheria vaccines. Preferred diphtheria toxoids are those prepared by formaldehyde treatment. The diphtheria toxoid can be obtained by growing C. diphtheriae in growth medium, followed by formaldehyde treatment, ultrafiltration and precipitation. The toxoided material may then be treated by a process comprising sterile filtration and/or dialysis. The diphtheria toxoid is preferably adsorbed onto an aluminum hydroxide adjuvant.

Tetanus toxoid: Clostridium tetani causes tetanus. Tetanus toxin can be treated to give a protective toxoid. The toxoids are used in tetanus vaccines. Preferred tetanus toxoids are those prepared by formaldehyde treatment. The tetanus toxoid can be obtained by growing C. tetani in growth medium, followed by formaldehyde treatment, ultrafiltration and precipitation. The material may then be treated by a process comprising sterile filtration and/or dialysis.

Hepatitis A virus antigens: Hepatitis A virus (HAV) is one of the known agents which causes viral hepatitis. A preferred HAV component is based on inactivated virus, and inactivation can be achieved by formalin treatment.

Hepatitis B virus (HBV) is one of the known agents which causes viral hepatitis. The major component of the capsid is a protein known as HBV surface antigen or, more commonly, HBsAg, which is typically a 226-amino acid polypeptide with a molecular weight of ~24 kDa. All existing hepatitis B vaccines contain HBsAg, and when this antigen is administered to a normal vaccinee it stimulates the production of anti-HBsAg antibodies which protect against HBV infection.

For vaccine manufacture, HBsAg has been made in two ways: purification of the antigen in particulate form from the plasma of chronic hepatitis B carriers or expression of the protein by recombinant DNA methods (e.g., recombinant expression in yeast cells). Unlike native HBsAg (i.e., as in the plasma-purified product), yeast-expressed HBsAg is generally non-glycosylated, and this is the most preferred form of HBsAg for use with the invention.

Conjugated Haemophilus influenzae type b antigens: Haemophilus influenzae type b (Hib) causes bacterial meningitis. Hib vaccines are typically based on the capsular saccharide antigen, the preparation of which is well documented. The Hib saccharide can be conjugated to a carrier protein in order to enhance its immunogenicity, especially in children. Typical carrier proteins are tetanus toxoid, diphtheria toxoid, CRM₁₉₇, H.influenzae protein D, and an outer membrane protein complex from serogroup B meningococcus. The saccharide moiety of the conjugate may comprise full-length polyribosylribitol phosphate (PRP) as prepared from Hib bacteria, and/or fragments of full-length PRP. Hib conjugates may or may not be adsorbed to an aluminum salt adjuvant.

### 2.5 Adjuvant(s)

In some embodiments, the immunogenic compositions disclosed herein may further comprise at least one, two or three adjuvants. In some embodiments, the immunogenic compositions disclosed herein may further comprise at least one adjuvant. In some embodiments, the immunogenic compositions disclosed herein may further comprise one adjuvant. In some embodiments, the immunogenic compositions disclosed herein may further comprise two adjuvants. The term “adjuvant” refers to a compound or mixture that enhances the immune response to an antigen. Antigens may act primarily as a delivery system, primarily as an immune modulator or have strong features of both.

Suitable adjuvants include those suitable for use in mammals, including humans. Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant. In a preferred embodiment, the immunogenic compositions disclosed herein comprise aluminum phosphate as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipaimitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068. In an embodiment of the present invention, the immunogenic compositions as disclosed herein comprise any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

In an embodiment of the present invention, the immunogenic compositions as disclosed herein comprise an A class CpG oligonucleotide. Preferably, the “A class” CpG oligonucleotide of the invention has the following nucleic acid sequence: 5′ GGGGACGACGTCGTGGGGGGG 3′ (SEQ ID NO: 1). Some non-limiting examples of A-Class oligonucleotides include: 5′ G*G*G_G_A_C_G_A_C_G_T_C_G_T_G_G*G*G*G*G*G 3′ (SEQ ID NO: 2); wherein refers to a phosphorothioate bond and “_” refers to a phosphodiester bond.

In an embodiment of the present invention, the immunogenic compositions as disclosed herein comprise a B class CpG Oligonucleotide. In one embodiment, the CpG oligonucleotide for use in the present invention is a B class CpG oligonucleotide represented by at least the formula: 5′ X₁X₂CGX₃X₄ 3′, wherein X₁, X₂, X₃, and X₄ are nucleotides. In one embodiment, X₂ is adenine, guanine, or thymine. In another embodiment, X₃ is cytosine, adenine, or thymine.

The B class CpG oligonucleotide sequences of the invention are those broadly described above as well as disclosed in WO 96/02555, WO 98/18810 and U.S. Pat. Nos. 6,194,388; 6,207,646; 6,214,806; 6,218,371; 6,239,116 and 6,339,068. Exemplary sequences include but are not limited to those disclosed in these latter applications and patents.

In an embodiment, the “B class” CpG oligonucleotide of the invention has the following nucleic acid sequence:

(SEQ ID NO: 3)
5′ TCGTCGTTTTTCGGTGCTTTT 3′,
or
(SEQ ID NO: 4)
5′ TCGTCGTTTTTCGGTCGTTTT 3′,
or
(SEQ ID NO: 5)
5′ TCGTCGTTTTGTCGTTTTGTCGTT 3′,
or
(SEQ ID NO: 6)
5′ TCGTCGTTTCGTCGTTTTGTCGTT 3′,
or
(SEQ ID NO: 7)
5′ TCGTCGTTTTGTCGTTTTTTTCGA 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

Some non-limiting examples of B-Class oligonucleotides include:

(SEQ ID NO: 8)
5′ T*C*G*T*C*G*T*T*T*T*T*C*G*G*T*G*C*T*T*T*T 3′,
or
(SEQ ID NO: 9)
5′ T*C*G*T*C*G*T*T*T*T*T*C*G*G*T*C*G*T*T*T*T 3′,
or
(SEQ ID NO: 10)
5′ T*C*G*T*C*G*T*T*T*T*G*T*C*G*T*T*T*T*G*T*C*G*T*
T 3′,
or
(SEQ ID NO: 11)
5′ T*C*G*T*C*G*T*T*T*C*G*T*C*G*T*T*T*T*G*T*C*G*T*
T 3′,
or
(SEQ ID NO: 12)
5′ T*C*G*T*C*G*T*T*T*T*G*T*C*G*T*T*T*T*T*T*T*C*G*
A 3′.

wherein “*” refers to a phosphorothioate bond.

In an embodiment of the present invention, the immunogenic compositions as disclosed herein comprise a C class CpG Oligonucleotide. In an embodiment, the “C class” CpG oligonucleotides of the invention have the following nucleic acid sequence:

(SEQ ID NO: 13)
5′ TCGCGTCGTTCGGCGCGCGCCG 3′,
or
(SEQ ID NO: 14)
5′ TCGTCGACGTTCGGCGCGCGCCG 3′,
or
(SEQ ID NO: 15)
5′ TCGGACGTTCGGCGCGCGCCG 3′,
or
(SEQ ID NO: 16)
5′ TCGGACGTTCGGCGCGCCG 3′,
or
(SEQ ID NO: 17)
5′ TCGCGTCGTTCGGCGCGCCG 3′,
or
(SEQ ID NO: 18)
5′ TCGACGTTCGGCGCGCGCCG 3′,
or
(SEQ ID NO: 19)
5′ TCGACGTTCGGCGCGCCG 3′,
or
(SEQ ID NO: 20)
5′ TCGCGTCGTTCGGCGCCG 3′,
or
(SEQ ID NO: 21)
5′ TCGCGACGTTCGGCGCGCGCCG 3′,
or
(SEQ ID NO: 22)
5′ TCGTCGTTTTCGGCGCGCGCCG 3′,
or
(SEQ ID NO: 23)
5′ TCGTCGTTTTCGGCGGCCGCCG 3′,
or
(SEQ ID NO: 24)
5′ TCGTCGTTTTACGGCGCCGTGCCG 3′,
or
(SEQ ID NO: 25)
5′ TCGTCGTTTTCGGCGCGCGCCGT 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide.

Some non-limiting examples of C-Class oligonucleotides include:

(SEQ ID NO: 26)
5′ T*C_G*C_G*T*C_G*T*T*C_G*G*C*G*C_G*C*G*C*C*G 3′,
or
(SEQ ID NO: 27)
5′ T*C_G*T*C_G*A*C_G*T*T*C_G*G*C*G*C_G*C*G*C*C*G
3′,
or
(SEQ ID NO: 28)
5′ T*C_G*G*A*C_G*T*T*C_G*G*C*G*C_G*C*G*C*C*G 3′,
or
(SEQ ID NO: 29)
5′ T*C_G*G*A*C_G*T*T*C_G*G*C*G*C*G*C*C*G 3′,
or
(SEQ ID NO: 30)
5′ T*C_G*C_G*T*C_G*T*T*C_G*G*C*G*C*G*C*C*G 3′,
or
(SEQ ID NO: 31)
5′ T*C_G*A*C_G*T*T*C_G*G*C*G*C_G*C*G*C*C*G 3′,
or
(SEQ ID NO: 32)
5′ T*C_G*A*C_G*T*T*C_G*G*C*G*C*G*C*C*G 3′,
or
(SEQ ID NO: 33)
5′ T*C_G*C_G*T*C_G*T*T*C_G*G*C*G*C*C*G 3′,
or
(SEQ ID NO: 34)
5′ T*C_G*C_G*A*C_G*T*T*C_G*G*C*G*C_G*C*G*C*C*G 3′,
or
(SEQ ID NO: 35)
5′ T*C*G*T*C*G*T*T*T*T*C*G*G*C*G*C*G*C*G*C*C*G 3′,
or
(SEQ ID NO: 36)
5′ T*C*G*T*C*G*T*T*T*T*C*G*G*C*G*G*C*C*G*C*C*G 3′,
or
(SEQ ID NO: 37)
5′ T*C*G*T*C_G*T*T*T*T*A*C_G*G*C*G*C*C_G*T*G*C*C*
G 3′,
or
(SEQ ID NO: 38)
5′ T*C_G*T*C*G*T*T*T*T*C*G*G*C*G*C*G*C*G*C*C*G*T
3′

wherein “*” refers to a phosphorothioate bond and “_” refers to a phosphodiester bond. In any of these sequences, an ethyl-uridine or a halogen may substitute for the 5′ T, examples of halogen substitutions include but are not limited to bromo-uridine or iodo-uridine substitutions.

In an embodiment of the present invention, the immunogenic compositions as disclosed herein comprise a P class CpG Oligonucleotide. In an embodiment, the CpG oligonucleotide for use in the present invention is a P class CpG oligonucleotide containing a 5′ TLR activation domain and at least two palindromic regions, one palindromic region being a 5′ palindromic region of at least 6 nucleotides in length and connected to a 3′ palindromic region of at least 8 nucleotides in length either directly or through a spacer, wherein the oligonucleotide includes at least one YpR dinucleotide. In an embodiment, said oligonucleotide is not T*C_G*T*C_G*A*C_G*T*T*C_G*G*C*G*C_G*C*G*C*C*G (SEQ ID NO: 27). In one embodiment the P class CpG oligonucleotide includes at least one unmethylated CpG dinucleotide. In another embodiment the TLR activation domain is TCG, TTCG, TTTCG, TYpR, TTYpR, TTTYpR, UCG, UUCG, UUUCG, TTT, or TTTT. In yet another embodiment the TLR activation domain is within the 5′ palindromic region. In another embodiment the TLR activation domain is immediately 5′ to the 5′ palindromic region. In an embodiment, the “P class” CpG oligonucleotides of the invention have the following nucleic acid sequence: 5′ TCGTCGACGATCGGCGCGCGCCG 3′ (SEQ ID NO: 39).

In said sequences, all of the linkages may be all phosphorothioate bonds. In another embodiment, one or more of the linkages may be phosphodiester, preferably between the “C” and the “G” of the CpG motif making a semi-soft CpG oligonucleotide. In any of these sequences, an ethyl-uridine or a halogen may substitute for the 5′ T, examples of halogen substitutions include but are not limited to bromo-uridine or iodo-uridine substitutions.

A non-limiting example of P-Class oligonucleotides include: 5′ T*C_G*T*C_G*A*C_G*A*T*C_G*G*C*G*C_G*C*G*C*C*G 3′ (SEQ ID NO: 40) wherein “*” refers to a phosphorothioate bond and “_” refers to a phosphodiester bond. In one embodiment the oligonucleotide includes at least one phosphorothioate linkage. In another embodiment all internucleotide linkages of the oligonucleotide are phosphorothioate linkages. In another embodiment the oligonucleotide includes at least one phosphodiester-like linkage. In another embodiment the phosphodiester-like linkage is a phosphodiester linkage. In another embodiment a lipophilic group is conjugated to the oligonucleotide. In one embodiment the lipophilic group is cholesterol.

In an embodiment, all the internucleotide linkages of the CpG oligonucleotides disclosed herein are phosphodiester bonds (“soft” oligonucleotides, as described in WO 2007/026190). In another embodiment, CpG oligonucleotides of the invention are rendered resistant to degradation (e.g., are stabilized). A “stabilized oligonucleotide” refers to an oligonucleotide that is relatively resistant to in vivo degradation (e.g., via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases.

The immunostimulatory oligonucleotides may have a chimeric backbone, which have combinations of phosphodiester and phosphorothioate linkages. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. When the phosphodiester linkage is preferentially located within the CpG motif such molecules are called “semi-soft” as described in WO 2007/026190.

Other modified oligonucleotides include combinations of phosphodiester, phosphorothioate, methylphosphonate, methylphosphorothioate, phosphorodithioate, and/or p-ethoxy linkages.

Mixed backbone modified ODN may be synthesized as described in WO 2007/026190.

The size of the CpG oligonucleotide (i.e., the number of nucleotide residues along the length of the oligonucleotide) also may contribute to the stimulatory activity of the oligonucleotide. For facilitating uptake into cells, CpG oligonucleotide of the invention preferably have a minimum length of 6 nucleotide residues. Oligonucleotides of any size greater than 6 nucleotides (even many kb long) are capable of inducing an immune response if sufficient immunostimulatory motifs are present, because larger oligonucleotides are degraded inside cells. In certain embodiments, the CpG oligonucleotides are 6 to 100 nucleotides long, preferentially 8 to 30 nucleotides long. In important embodiments, nucleic acids and oligonucleotides of the invention are not plasmids or expression vectors.

In an embodiment, the CpG oligonucleotide disclosed herein comprise substitutions or modifications, such as in the bases and/or sugars as described at paragraphs 134 to 147 of WO 2007/026190.

In an embodiment, the CpG oligonucleotide of the present invention is chemically modified. Examples of chemical modifications are known to the skilled person and are described, for example in Uhlmann et al. (1990) Chem. Rev. 90:543; S. Agrawal, Ed., Humana Press, Totowa, USA 1993; Crooke et al. (1996) Annu. Rev. Pharmacol. Toxicol. 36:107-129; and Hunziker et al. (1995) Mod. Synth. Methods 7:331-417. An oligonucleotide according to the invention may have one or more modifications, wherein each modification is located at a particular phosphodiester internucleoside bridge and/or at a particular p-D-ribose unit and/or at a particular natural nucleoside base position in comparison to an oligonucleotide of the same sequence which is composed of natural DNA or RNA.

In some embodiments of the invention, CpG-containing nucleic acids might be simply mixed with immunogenic carriers according to methods known to those skilled in the art (see, e.g., WO 03/024480).

In a particular embodiment of the present invention, any of the immunogenic compositions disclosed herein comprise from 2 μg to 100 mg of CpG oligonucleotide. In a particular embodiment of the present invention, the immunogenic composition of the invention comprises 0.1 mg to 50 mg of CpG oligonucleotide, preferably from 0.2 mg to 10 mg CpG oligonucleotide, more preferably from 0.3 mg to 5 mg CpG oligonucleotide.

In a particular embodiment of the present invention, the immunogenic composition of the invention comprises from 0.3 mg to 5 mg CpG oligonucleotide. Even preferably, the immunogenic composition of the invention may comprise from 0.5 to 2 mg CpG oligonucleotide. Most preferably, the immunogenic composition of the invention may comprise from 0.75 to 1.5 mg CpG oligonucleotide. In a preferred embodiment, any of the immunogenic composition disclosed herein may comprise about 1 mg CpG oligonucleotide.

### 3 Formulation

The immunogenic compositions of the invention may be formulated in liquid form (i.e., solutions or suspensions) or in a lyophilized form. In an embodiment, the immunogenic composition of the invention is formulated in a liquid form. In an embodiment, the immunogenic composition of the invention is formulated in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In some embodiments, the buffer is succinate. In some embodiments, the buffer is histidine. In certain embodiments, the buffer is succinate at a final concentration of 1 mM to 10 mM.

In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR®EL), soy lecithin and a poloxamer.

In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0.

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL. In an embodiment, the immunogenic composition of the invention for injection has a volume of 0.2 mL to 1 mL, even more preferably a volume of about 0.5 mL.

### 4 Uses of the Glycoconjugate and Immunogenic Compositions of the Invention

The glycoconjugates disclosed herein may be use as antigens. For example, they may be part of a vaccine.

Therefore, in an embodiment, the immunogenic compositions of the invention are for use as a medicament.

In an embodiment, the immunogenic compositions of the invention are for use as a vaccine.

Therefore, in an embodiment, the immunogenic compositions described herein are for use in generating an immune response in a subject. In one aspect, the subject is a mammal, such as a human, non-human primate, cat, sheep, pig, horse, bovine or dog. In one aspect, the subject is a human.

The immunogenic compositions described herein may be used in therapeutic or prophylactic methods for preventing, treating or ameliorating a bacterial infection, disease or condition in a subject. Thus, in one aspect, the disclosure provides a method of preventing, treating or ameliorating an infection, disease or condition associated with a bacterial infection in a subject, comprising administering to the subject an immunologically effective amount of an immunogenic composition of the disclosure.

The immunogenic composition of the present disclosure can be used to protect or treat a human susceptible to a bacterial infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

### 5 Subject to be Treated with the Immunogenic Compositions of the Invention

As disclosed herein, the immunogenic compositions described herein may be used in various therapeutic or prophylactic methods for preventing, treating or ameliorating a bacterial infection, disease or condition in a subject.

In a preferred embodiment, said subject is a human. In a most preferred embodiment, said subject is a newborn (i.e., under three months of age), an infant (i.e., from 3 months to one year of age) or a toddler (i.e., from one year to four years of age). In an embodiment, the immunogenic compositions disclosed herein are for use as a vaccine.

In such embodiment, the subject to be vaccinated may be less than 1 year of age. For example, the subject to be vaccinated can be about 1, about 2, about 3, about 4, about 5, about 6, about 7, about 8, about 9, about 10, about 11 or about 12 months of age. In an embodiment, the subject to be vaccinated is about 2, about 4 or about 6 months of age. In another embodiment, the subject to be vaccinated is less than 2 years of age.

For example, the subject to be vaccinated can be about 12 to about 15 months of age. In some cases, as little as one dose of the immunogenic composition according to the invention is needed, but under some circumstances, a second, third or fourth dose may be given (see section 8 below).

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

In an embodiment of the present invention, the immunocompromised subject to be vaccinated suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is selected from the one disclosed on page 24, line 11, to page 25, line 19, of WO 2010/125480.

In a particular embodiment of the present invention, the immunocompromised subject to be vaccinated suffers from a disease selected from the groups consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised subject to be vaccinated suffers from malnutrition.

In a particular embodiment of the present invention, the immunocompromised subject to be vaccinated is taking a drug or treatment that lowers the body's resistance to infection. In an embodiment, said drug is selected from the one disclosed on page 26, line 33, to page 26, line 4, of WO 2010/125480.

In a particular embodiment of the present invention, the immunocompromised subject to be vaccinated is a smoker.

In a particular embodiment of the present invention, the immunocompromised subject to be vaccinated has a white blood cell count (leukocyte count) below 5×10⁹ cells per liter, or below 4×10⁹ cells per liter, or below 3×10⁹ cells per liter, or below 2×10⁹ cells per liter, or below 1×10⁹ cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised subject to be vaccinated suffers from neutropenia. In a particular embodiment of the present invention, the immunocompromised subject to be vaccinated has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter.

A low white blood cell count or “neutropenia” is a condition characterized by abnormally low levels of neutrophils in the circulating blood. Neutrophils are a specific kind of white blood cell that help to prevent and fight infections. The most common reason that cancer patients experience neutropenia is as a side effect of chemotherapy.

Chemotherapy-induced neutropenia increases a patient's risk of infection and disrupts cancer treatment.

In a particular embodiment of the present invention, the immunocompromised subject to be vaccinated has a CD4+ cell count below 500/mm³, or CD4+ cell count below 300/mm³, or CD4+ cell count below 200/mm³, CD4+ cell count below 100/mm³, CD4+ cell count below 75/mm³, or CD4+ cell count below 50/mm³.

CD4 cell tests are normally reported as the number of cells in mm³. Normal CD4 counts are between 500 and 1,600, and CD8 counts are between 375 and 1,100. CD4 counts drop dramatically in people with HIV.

In an embodiment of the invention, any of the immunocompromised subjects disclosed herein is a human male. In an embodiment of the invention, any of the immunocompromised subjects disclosed herein is a human female.

### 6 Regimen

In some cases, as little as one dose of the immunogenic composition according to the invention is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the invention is a single dose. In a particular embodiment, said single dose schedule is for healthy persons being at least 2 years of age.

In an embodiment, the schedule of vaccination of the immunogenic composition according to the invention is a multiple dose schedule. In a particular embodiment, said multiple dose schedule consists of a series of 2 doses separated by an interval of about 1 month to about 2 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses separated by an interval of about 1 month to about 2 months. In another embodiment, said multiple dose schedule consists of a series of 3 doses separated by an interval of about 1 month, or a series of 3 doses separated by an interval of about 2 months.

In another embodiment, said multiple dose schedule consists of a series of 3 doses separated by an interval of about 1 month to about 2 months followed by a fourth dose about 10 months to about 13 months after the first dose. In another embodiment, said multiple dose schedule consists of a series of 3 doses separated by an interval of about 1 month followed by a fourth dose about 10 months to about 13 months after the first dose, or a series of 3 doses separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses separated by an interval of about 1 month to about 2 months (for example 28-56 days between doses), starting at 2 months of age, and followed by a toddler dose at 12-18 months of age. In an embodiment, said multiple dose schedule consists of a series of 3 doses separated by an interval of about 1 month to about 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more boosts are given at intervals that range from about 2 to about 24 weeks, preferably with a dosing interval of 4-8 weeks.

In an embodiment, a prime dose is given at day 0 and a boost is given about 3 months later.

### 7. The Invention Also Provides the Following Embodiments as Defined in the Following Numbered Paragraphs 1 to 48

1. A method of making a capsular saccharide glycoconjugate, comprising the steps of:
   (a) contacting an isolated Streptococcus pneumoniae capsular saccharide with an azido linker and 4-(4,6-dimethoxy [1,3,5]triazin-2-yl)-4-methyl-morpholinium (DMTMM) to produce an azido incorporated saccharide;
   (b) contacting a carrier protein with an agent bearing an N-hydroxysuccinimide (NHS) moiety and an alkyne group to produce an alkyne functionalized carrier protein; and
   (c) reacting the azido incorporated saccharide with the alkyne functionalized carrier protein by Cu⁺¹ mediated azide-alkyne cycloaddition reaction,
   thereby forming the capsular saccharide glycoconjugate.
2. The method of paragraph 1, wherein the Streptococcus pneumoniae is selected from the group consisting of Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.
3. The method of any one of paragraphs 1-2, wherein the azido linker is a compound of formula (I):

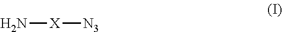

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.
4. The method of any one of paragraphs 1-2, wherein the azido linker is a compound of formula (II):

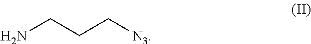

5. The method of any one of paragraphs 1-4, wherein the agent bearing an N-Hydroxysuccinimide (NHS) moiety and an alkyne group is an agent bearing an N-Hydroxysuccinimide (NHS) moiety and a terminal alkyne.
6. The method of any one of paragraphs 1-4, wherein the agent bearing an N-Hydroxysuccinimide (NHS) moiety and an alkyne group is a compound of formula (III):

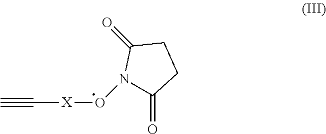

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.
7. The method of any one of paragraphs 1-4, wherein the agent bearing an N-Hydroxysuccinimide (NHS) moiety and an alkyne group is a compound of formula (IV):

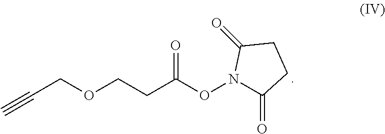 [SMILES: C#CCOCCC(=O)ON1C(=O)CCC1=O]

8. The method of any one of paragraphs 1-7, wherein the carrier protein is selected from the group consisting of CRM₁₉₇, Streptococcus C5a peptidase (SCP), diphtheria toxoid (DT), tetanus toxoid (TT), meningococcal outer membrane protein (OMP) complex, non-typeable Haemophilus influenzae protein D, and pan HLA DR-binding epitope (PADRE).
9. The method of any one of paragraphs 1-8, wherein the capsular saccharide glycoconjugate has a molecular weight of >1000 kDA.
10. The method of any one of paragraphs 1-9, wherein the capsular saccharide glycoconjugate has a free saccharide % of <30%.
11. The method of any one of paragraphs 1-10, wherein the capsular saccharide glycoconjugate has a conjugate saccharide to protein ratio of 0.5-2.5.
12. The method of any one of paragraphs 1-11, wherein the azido linker is present in an amount that is between 0.01-10 molar equivalent to an amount of polysaccharide Repeat Unit of the azido incorporated saccharide.
13. The method of any one of paragraphs 1-12, wherein the agent bearing an N-Hydroxysuccinimide (NHS) moiety and an alkyne group is present in an amount that is between 0.1-10 molar equivalents to an amount of lysines on the carrier protein.
14. The method of any one of paragraphs 1-13, wherein step (c) is carried out in an aqueous buffer in the presence of copper (I) as catalyst.
15. The method of any one of paragraphs 1-14, further comprising purifying the capsular saccharide glycoconjugate after step (c).
16. A capsular saccharide glycoconjugate produced according to any one of the methods of paragraphs 1 to 15.
17. A method of making a capsular saccharide glycoconjugate, comprising the steps of:
   (a) contacting an isolated Streptococcus pneumoniae capsular saccharide with 1-cyano-4-dimethylaminopyridinium tetrafluoroborate (CDAP) to produce a reactive intermediate;
   (b) contacting the reactive intermediate with an azido linker to produce an azido incorporated saccharide;
   (c) contacting a carrier protein with an agent bearing an N-hydroxysuccinimide (NHS) moiety and an alkyne group to produce an alkyne functionalized carrier protein; and
   (d) reacting the azido incorporated saccharide with the alkyne functionalized carrier protein by Cu⁺¹ mediated azide-alkyne cycloaddition reaction,
   thereby forming the capsular saccharide glycoconjugate.
18. The method of paragraph 17, wherein the Streptococcus pneumoniae is selected from the group consisting of Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.
19. The method of any one of paragraphs 17-18, wherein the azido linker is a compound of formula (I):

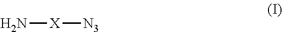

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.
20. The method of any one of paragraphs 17-18, wherein the azido linker is a compound of formula (II):

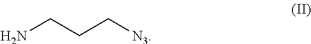

21. The method of any one of paragraphs 17-20, wherein the agent bearing an N-Hydroxysuccinimide (NHS) moiety and an alkyne group is an agent bearing an N-Hydroxysuccinimide (NHS) moiety and a terminal alkyne.
22. The method of any one of paragraphs 17-20, wherein the agent bearing an N-Hydroxysuccinimide (NHS) moiety and an alkyne group is a compound of formula (III):

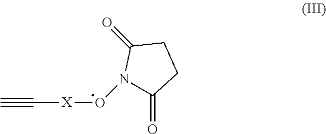

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.
23. The method of any one of paragraphs 17-20, wherein the agent bearing an N-Hydroxysuccinimide (NHS) moiety and an alkyne group is a compound of formula (IV):

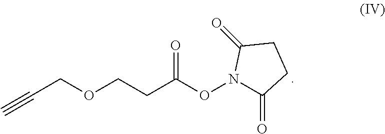 [SMILES: C#CCOCCC(=O)ON1C(=O)CCC1=O]

24. The method of any one of paragraphs 17-23, wherein the carrier protein is selected from the group consisting of CRM₁₉₇, Streptococcus C5a peptidase (SCP), diphtheria toxoid (DT), tetanus toxoid (TT), meningococcal outer membrane protein (OMP) complex, non-typeable Haemophilus influenzae protein D, and pan HLA DR-binding epitope (PADRE).
25. The method of any one of paragraphs 17-24, wherein the capsular saccharide glycoconjugate has a molecular weight of >1000 kDA.
26. The method of any one of paragraphs 17-25, wherein the capsular saccharide glycoconjugate has a free saccharide % of <30%.
27. The method of any one of paragraphs 17-26, wherein the capsular saccharide glycoconjugate has a conjugate saccharide to protein ratio of 0.5-2.5.
28. The method of any one of paragraphs 17-27, wherein the azido linker is present in an amount that is between 0.01-10 molar equivalent to an amount of polysaccharide Repeat Unit of the azido incorporated saccharide.
29. The method of any one of paragraphs 17-28, wherein the agent bearing an N-Hydroxysuccinimide (NHS) moiety and an alkyne group is present in an amount that is between 0.1-10 molar equivalents to an amount of lysines on the carrier protein.
30. The method of any one of paragraphs 17-29, wherein step (c) is carried out in an aqueous buffer in the presence of copper (I) as catalyst.
31. The method of any one of paragraphs 17-30, further comprising purifying the capsular saccharide glycoconjugate after step (c).
32. A capsular saccharide glycoconjugate produced according to any one of the methods of paragraphs 17 to 31.
33. A method of making a capsular saccharide glycoconjugate, comprising the steps of:
   (a) contacting a primary alcohol of an isolated Streptococcus pneumoniae capsular saccharide with 4-Acetamido-2,2,6,6-tetramethyl-1-oxopiperidinium tetrafluoroborate (TEMPOX) to produce a reactive saccharide intermediate;
   (b) contacting the reactive saccharide intermediate with an azido linker to form an imine bond and produce an azido saccharide intermediate;
   (c) contacting the azido saccharide intermediate with sodium cyanoborohydride (NaBH₃CN) to reduce the imine bond to an amine bond and produce a reduced azido incorporated saccharide;
   (d) contacting a carrier protein with an agent bearing an N-hydroxysuccinimide (NHS) moiety and an alkyne group to produce an alkyne functionalized carrier protein; and
   (e) reacting the reduced azido incorporated saccharide with the alkyne functionalized carrier protein by Cu⁺¹ mediated azide-alkyne cycloaddition reaction,
   thereby forming the capsular saccharide glycoconjugate.
34. The method of paragraph 33, wherein the Streptococcus pneumoniae is selected from the group consisting of Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.
35. The method of any one of paragraphs 33-34, wherein the azido linker is a compound of formula (I):

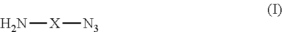

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.
36. The method of any one of paragraphs 33-34, wherein the azido linker is a compound of formula (II):

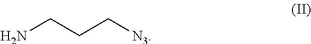

37. The method of any one of paragraphs 33-36, wherein the agent bearing an N-Hydroxysuccinimide (NHS) moiety and an alkyne group is an agent bearing an N-Hydroxysuccinimide (NHS) moiety and a terminal alkyne.
38. The method of any one of paragraphs 33-36, wherein the agent bearing an N-Hydroxysuccinimide (NHS) moiety and an alkyne group is a compound of formula (III):

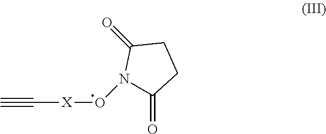

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.
39. The method of any one of paragraphs 33-36, wherein the agent bearing an N-Hydroxysuccinimide (NHS) moiety and an alkyne group is a compound of formula (IV):

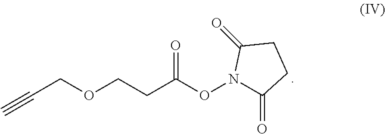 [SMILES: C#CCOCCC(=O)ON1C(=O)CCC1=O]

40. The method of any one of paragraphs 33-39, wherein the carrier protein is selected from the group consisting of CRM₁₉₇, Streptococcus C5a peptidase (SCP), diphtheria toxoid (DT), tetanus toxoid (TT), meningococcal outer membrane protein (OMP) complex, non-typeable Haemophilus influenzae protein D, and pan HLA DR-binding epitope (PADRE).
41. The method of any one of paragraphs 33-40, wherein the capsular saccharide glycoconjugate has a molecular weight of >1000 kDA.
42. The method of any one of paragraphs 33-41, wherein the capsular saccharide glycoconjugate has a free saccharide % of <30%.
43. The method of any one of paragraphs 33-42, wherein the capsular saccharide glycoconjugate has a conjugate saccharide to protein ratio of 0.5-2.5.
44. The method of any one of paragraphs 33-43, wherein the azido linker is present in an amount that is between 0.01-10 molar equivalent to an amount of polysaccharide Repeat Unit of the azido incorporated saccharide.
45. The method of any one of paragraphs 33-44, wherein the agent bearing an N-Hydroxysuccinimide (NHS) moiety and an alkyne group is present in an amount that is between 0.1-10 molar equivalents to an amount of lysines on the carrier protein.
46. The method of any one of paragraphs 33-45, wherein step (c) is carried out in an aqueous buffer in the presence of copper (I) as catalyst.
47. The method of any one of paragraphs 33-46, further comprising purifying the capsular saccharide glycoconjugate after step (c).
48. A capsular saccharide glycoconjugate produced according to any one of the methods of paragraphs 33 to 47.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLES

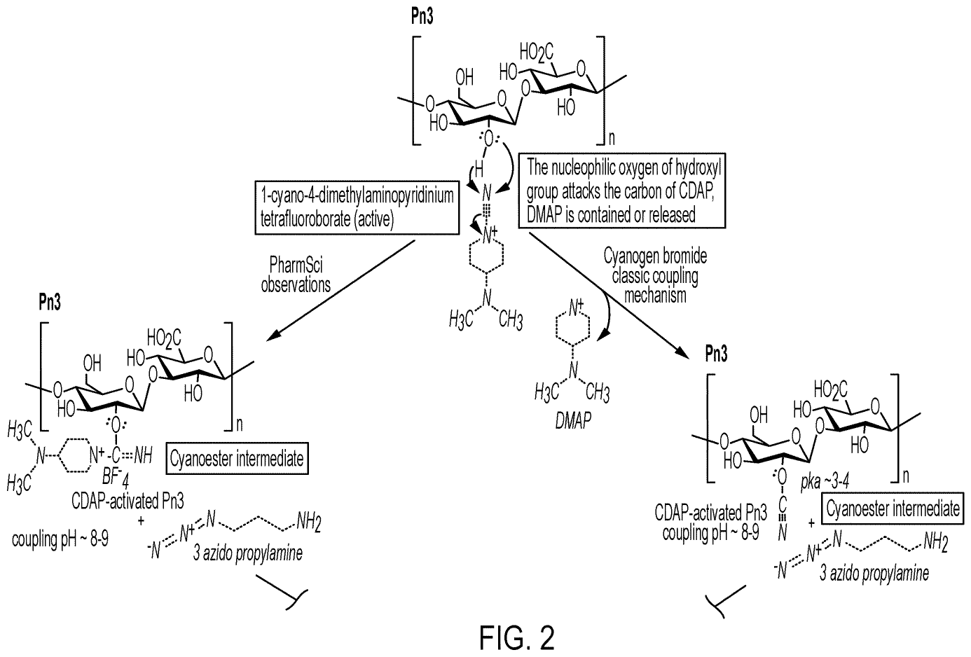

### Example 1: CDAP Activation and Incorporation of 3-azidopropylamine of S. Pneumoniae Serotype 3 Capsular Polysaccharide Using Click Chemistry (See FIG. 2)

1. Activation of Serotype 3 Capsular Polysaccharide with Azido Linker

In the presence of 1-cyano-4-dimethylaminopyridinium tetrafluroborate (CDAP), the nucleophilic oxygen of the Pn3 hydroxyl group attacks the carbon of CDAP which couples a cyano group to the nucleophilic oxygen and creates a leaving group of 4-Dimethylaminopyridine (DMAP) [Lees et al. (1996) Vaccine 14(3):190-198]. This exchange of the cyano group at the targeted hydroxyl site creates a cyanoester intermediate which is highly reactive. The cyanoester intermediate then reacts under basic conditions with the amino group of 3-azidopropylamine to form a stable O-alkyl-isourea linkage. The resulting intermediate is the activated Pn3 polysaccharide referred to as Pn3CDAP. The mechanism of Pn3CDAP activation and incorporation is depicted in FIG. 2, as well as a proposed alternative mechanism for CDAP activation which deviates from the classic cyanogen bromide coupling mechanism. The alternative mechanism suggests that the DMAP group combines with the cyanoester intermediate and then the 3-azidopropylamine amine group would attack the carbon adjacent to the DMAP group and subsequently displace the DMAP group resulting in a stable O-alkyl-isourea linkage [Kuzmenko et al. (2013) Materials Science and Engineering 33(8):4599-4607].

The azide incorporated polysaccharide species, Pn3CDAP, was developed using a cyanylating reagent (CDAP, 2:1 mass equivalent CDAP to polysaccharide) to activate the Pn3 polysaccharide and conjugate 3-azidopropylamine (1:1 mass equivalent 3-azidopropylamine to polysaccharide) to the activated Pn3 polysaccharide. Modifications to the process included: replacing the buffer:CDAP steps of Triethylamine buffer (TEA) buffer for sodium bicarbonate buffer, ranging the polysaccharide concentration (0.5-2 mg/ml) in the reaction to vary the azide content, and removing the saline addition step to the polysaccharide prior to adding CDAP.

2. Replacing TEA Buffer with Sodium Bicarbonate Buffer

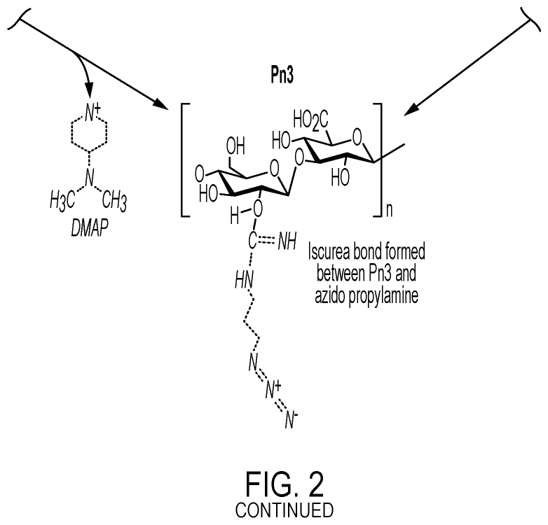

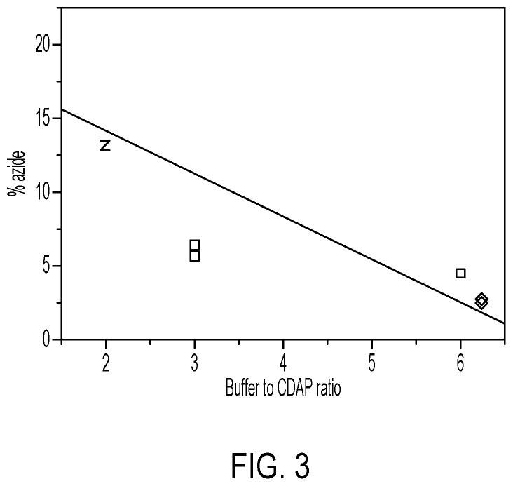

To avoid potential toxicity concerns of TEA buffer (including moderate acute toxicity from inhalation, moderate to high acute toxicity from oral exposure, and high acute toxicity from dermal exposure), 0.2 M TEA at pH 11.9 was replaced with 0.8 M sodium bicarbonate buffer at pH 9.5. The variation in the buffer strength and capacity required a new buffer:CDAP ratio. Experiments were conducted to determine a model for 3-azidopropylamine incorporation based on sodium biocarbonate buffer:CDAP ratio (2-6.25). Azide incorporation from 2.4-13.1% into Pn3 polysaccharide over biocarbonate buffer:CDAP ratios (vol:vol) ranging from 2-6.25 was achieved (FIG. 3). The use of a buffer with a higher ionic strength also allowed for the titration of the CDAP activated species without over diluting the reaction which could limit the extent of 3-azidopropylamine incorporation due to a depressed kinetic coupling rate. The addition of bicarbonate buffer to the CDAP activated polysaccharide raises the pH of the reaction prior to the addition of the azidopropylamine. This increase in pH is necessary to ensure enough amine groups on the 3-azidopropylamine are deprotonated and therefore are available to couple with the cyanoester-activated polysaccharide. However, the cyanoester intermediate itself is unstable at high pH and over time can hydrolyze. FIG. 3 demonstrates that adding too much bicarbonate buffer (pH 9.5) to the reaction prior to adding the azidopropylamine causes a decrease in overall coupling efficiency resulting in low azide incorporation values.

### Example 2: S. pneumoniae Serotype 3 Capsular Polysaccharide DMTMM Activation and Incorporation

### 1. DMTMM Activation and 3-Azidopropylamine Incorporation

The azide incorporated polysaccharide species, Pn3DMTMM, was developed using 4-(4,6-dimethoxy [1,3,5]triazin-2-yl)-4-methyl-morpholinium (DMTMM, 0.11-0.58 mass equivalent polysaccharide:DMTMM) to activate the Pn3 polysaccharide and conjugate 3-azidopropylamine (1:0.5 mass equivalent polysaccharide:amine) to the activated Pn3 polysaccharide. DMTMM is a zero-length crosslinker which eliminates any potential immune response from the crosslinking structure of the vaccine construct. N-substituted carbodiimides are widely used for peptide incorporation. Activation of carboxylic acids with carbodiimides (such as EDC) is complicated by hydrolysis and irreversible intramolecular rearrangement of the desired reactive o-acylisourea derivative to an unreactive N-acylurea derivative. Newer reagents such as DMTMM overcome this complication using a one pot formation of a highly stable super active ester.

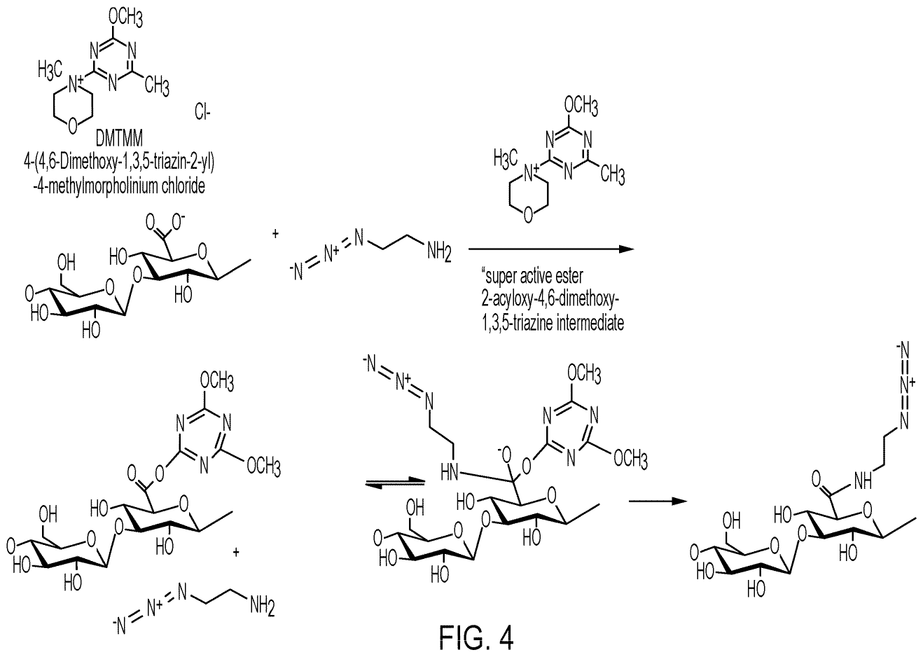

As shown in FIG. 4, DMTMM targets the carboxylic acid group of Pn3 polysaccharide through a nucleophilic attack by triazine to form active Pn3DMTMM. The morpholinium group of DMTMM, NMM, serves as a leaving group when a nucleophilic aromatic substitution takes place [Montalbetti and Falque (2005) Tetrahedron 61(46):10827-10852]. An ester bond is formed between Pn3 and DMTMM. The resulting molecule is referred to as an “super active ester” (2-acyloxy-4,6-dimethoxy-1,3,5-triazine) due to its ability to react further and form amides and esters.

In the second step of the reaction, a nucleophilic amine (demonstrated here with 3-azidopropylamine) attacks the carboxylic carbon on the active ester to form an amide bond as present in FIG. 4. The amine is present in the solution during the formation of the super active ester. This deviates from traditional EDC process which creates an unstable O-acylisourea via EDC carbocation incorporation at the carboxyl acid site of the target molecule. This O-acylisourea can be converted to various side products (Urea, N-Acylurea, and Acid Anhydride) which impacts the overall efficiency of EDC coupling chemistry. The “super active ester” evades these side reactions providing a super-efficient and green coupling chemistry. The insoluble hydroxytriazine by-product is formed and can be removed by filtration easily.

### Example 3. TEMPO/NCS and TEMPOX Oxidation of Pn3 Polysaccharide

Oxidation of alcohols to aldehyde groups via nitroxyl or nitroxide radicals allows chemical modification of primary alcohols on polysaccharides [Bragd et al. (2004) Topics in Catalysis 27:49-66]. 2,2,6,6-tetramethylpiperidine-noxyl (TEMPO) and its derivatives belong to a family of cyclic nitroxyl and nitroxide radicals. For TEMPO mediated oxidation of primary alcohols on polysaccharides, one of two methods are used. Polysaccharides are either 1) oxidized by catalytic amounts of the nitrosonium salt that are generated in situ by primary oxidation of the nitroxyl radical via chlorine or bromine or 2) are directly oxidized by adding stoichiometric amounts of the nitrosonium ion salt. In TEMPO/NCS mediated oxidation, the nitrosonium ion salt is generated through oxidation of the TEMPO radical by NCS. Throughout the reaction, the NCS is consumed and the TEMPO radical is regenerated.

TEMPO/NCS oxidation of Pn3 polysaccharide was observed to proportionally reduce Pn3 polysaccharide molecular weight with increasing NCS molar equivalency. Briefly, native Pn3 polysaccharide was diluted and mixed in sodium carbonate buffer pH 8.6 at a temperature of 23±2° C. TEMPO was added at a final molar equivalent (Meq) of 0.074 with respect to the polysaccharide. Within 15 minutes of the TEMPO addition, the oxidation reaction began with the addition of NCS for a final Meq of 0.6, 1.0 and 4.3 with respect to the polysaccharide. The oxidation reaction continued for 120±15 minutes.

The reaction was quenched via the addition of n-propanol at a final 50±10 Meq with respect to the polysaccharide for a target 90±30 minutes. The oxidized polysaccharide was purified by ultrafiltration/diafiltration (UFDF) against chilled water for injection (WFI) using a 30 kDa molecular weight cut-off (MWCO) polyethersulfone (PES) tangential flow filtration (TFF) membrane. Purified polysaccharide was filtered using a 0.45/0.22 μm filter and stored at 2-8° C. Characterization results for Pn3 polysaccharide oxidized with TEMPO/NCS are in Table 1. Since NCS Meq is the primary driver for the oxidation reaction, it is expected that the condition with the highest NCS Meq has both the lowest molecular weight and is the most chemically modified.

TABLE 1
TEMPO/NCS Oxidation of Serotype Pn3 Polysaccharide

| Sample | | Molecular Weight | Degree of Oxidation |
|---|---|---|---|
| ID | NCS Meq | (kDa) | (DO) |
| 1 | 0.6 | NT | 4.6 |
| 2 | 1.0 | 248 | 4.5 |
| 3 | 4.3 | 169 | 2.6 |

Previous work with serotype 12F polysaccharide also demonstrated that the molecular weight of the 12F polysaccharide decreases during TEMPO/NCS oxidation in an NCS dependent fashion. Potential mechanisms include a-chlorination and free radical generation leading to chain scission of the polysaccharide.

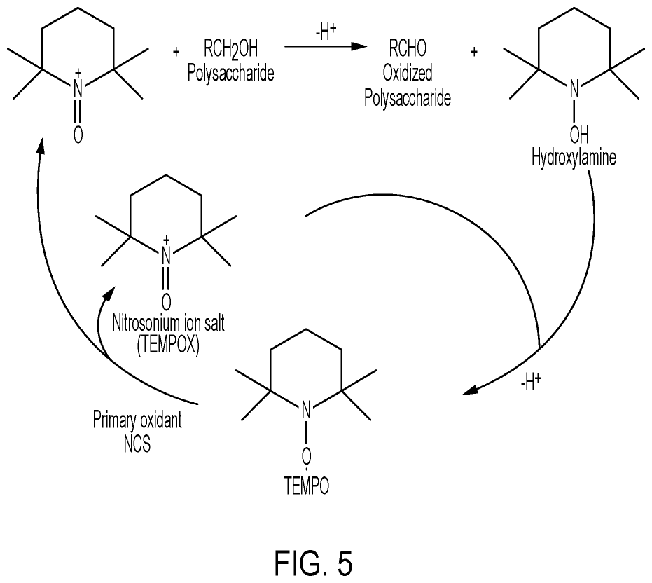

To avoid NCS-induced molecular weight reduction, direct oxidation of the serotype 3 polysaccharide by the nitrosonium ion salt 4-Acetamido-2,2,6,6-tetramethyl-1-oxopiperidinium tetrafluoroborate (TEMPOX) was also performed. In this mechanism, as shown in in FIG. 5, TEMPOX is added directly to the polysaccharide and is consumed during the reaction. The resulting activated Pn3 polysaccharide intermediate from TEMPOX oxidation is referred to as Pn3TEMPOX.

In this work the Pn3 polysaccharide size was first reduced prior to oxidation by acid hydrolysis. Briefly, native Pn3 polysaccharide was thawed and diluted to a final concentration of 2 mg/ml in 0.2M acetate buffer. The reaction was then heated to 85° C. for 60±15 minutes with ramp up/down times targeting s 90 minutes. Following cool down, the polysaccharide was immediately purified by UFDF against 20 diavolumes WFI using 100 kDa MWCO PES TFF membranes. The molecular weight of the hydrolyzed polysaccharide was determined by SEC-MALLs and then diluted to a final concentration of 1 mg/ml in bicarbonate buffer pH 8.6. The hydrolyzed polysaccharide was oxidized by 0.2-1.9 Meq TEMPOX for 120 or 1037 minutes as described in Table 2. Following oxidation, TEMPOX was quenched by the addition of n-propanol and the activated polysaccharides were purified by UFDF against WFI using 30 kDa PES TFF membranes. The oxidized polysaccharides were 0.45/0.22 μm filtered and characterization results are shown in Table 2.

TABLE 2
TEMPOX Oxidation of Serotype Pn3 Polysaccharide

| | | Hydro- | | Oxi- | | Degree |
|---|---|---|---|---|---|---|
| | | lyzed | | dation | Oxidized | of Oxi- |
| Sample | Scale | MW | TEMPOX | Time | Poly MW | dation |
| ID | (L) | (kDa) | Meq | (min) | (kDa) | (DO) |
| 5A | 0.1 | 822 | 0.26 | 120 | Not tested | 299 |
| 5B | 0.1 | 784 | 0.43 | 120 | 823 | 93 |
| 5C | 0.1 | 767 | 0.60 | 120 | 730 | 76 |
| 6 | 0.1 | 825 | 0.19 | 1037 | 460 | 21 |
| 7 | 0.1 | 758 | 0.96 | 1037 | 223 | 6 |
| 8 | 0.1 | 811 | 1.92 | 1037 | 347 | 3 |
| 9 | 2 | 917 | 1 | 1080 | 25 | 2 |
| 14 | 2 | 1285 | 0.2 | 1020 | 42 | 8 |

During the first 2 hours of the TEMPOX oxidation reaction, the molecular weight of the polysaccharide remained essentially unchanged and the activation level of the polysaccharide level remained low as shown by the high degree of oxidation. Extending the reaction to 17 hours produced polysaccharide with high degrees of activation (DO<10) depending upon the TEMPOX Meq. However, even in the absence of NCS, a significant reduction in molecular weight during the reaction was observed with increasing TEMPOX meq. and reaction time.

It was hypothesized that a competing side reaction (base-catalyzed beta elimination) leading to polysaccharide chain scission was occurring during TEMPOX activation, and that reaction pH and reaction temperature were the most likely parameters affecting the kinetics of this side reaction. An experiment was next performed to optimize reaction parameters in order to achieve the target Pn3 polysaccharide degree of oxidation without a large decrease in molecular weight. Conditions for the experimental runs are shown in Table 3. The target polysaccharide concentration in the each of the reactions was 1.25 mg/ml.

TABLE 3
Conditions for TEMPOX Oxidation
of Serotype Pn3 Polysaccharide

| Sample | DOE pattern (pH, | | TEMPOX | Temp |
|---|---|---|---|---|
| ID | TEMPOX, Temp) | pH | Meq | (° C.) |
| 21-4 | +−+ | 8.5 | 0.1 | 23 |
| 21-5 | −++ | 7.5 | 0.4 | 23 |
| 21-6 | −−+ | 7.5 | 0.1 | 23 |
| 21-7 | +−− | 8.5 | 0.1 | 5 |
| 21-8 | ++− | 8.5 | 0.4 | 5 |
| 21-9 | +++ | 8.5 | 1.4 | 23 |
| 21-10 | 0 | 8 | 0.25 | 14 |
| 21-11 | −+− | 7.5 | 0.4 | 5 |
| 21-12 | −−− | 7.5 | 0.1 | 5 |

SEC-MALLS MW and Degree of Oxidation results for the runs are shown in Table 4. The starting molecular weight for the hydrolyzed Pn3 polysaccharide was 967 kDa.

TABLE 4
Results for TEMPOX Oxidation of Serotype Pn3 Polysaccharide

| | Oxidized Polysaccharide Molecular | Degree of |
|---|---|---|
| Sample ID | Weight (kDa) | Oxidation |
| 21-4 | 158 | 11 |
| 21-5 | 216 | 4 |
| 21-6 | 345 | 13 |
| 21-7 | 232 | 9 |
| 21-8 | 169 | 3 |
| 21-9 | 245 | 5 |
| 21-10 | 326 | 5 |
| 21-11 | 495 | 4 |
| 21-12 | 597 | 11 |

It was found that overall, Meq strongly drives 1/DO (aldehye percentage while pH and temperature significantly contribute to the change in molecular weight. Reaction parameter targets of 0.1 Meq TEMPOX, pH 7.5, and a reaction temperature of 5° C. were identified as the optimal conditions.

Table 5 shows the results for runs under these optimized conditions. The starting molecular weight for the hydrolyzed Pn3 polysaccharide was 967 kDa and the target polysaccharide concentration in the each of the reactions was 1.25 mg/ml. The oxidized polysaccharides were purified as described above. As shown in Table 5, the DO results were near the target of 10, whereas the molecular weight of the polysaccharide did not change significantly during the oxidation.

TABLE 5
Scale-up of Optimized TEMPOX Oxidation
of Serotype Pn3 Polysaccharide

| | Hydrolyzed | | | | Oxidized | Degree of |
|---|---|---|---|---|---|---|
| Sample | MW | TEMPOX | Temp | | Poly MW | Oxidation |
| ID | (kDa) | Meq | (° C.) | pH | (kDa) | (DO) |
| 11-2 | 967 | 0.1 | 5 | 7.5 | 1137 | 11 |
| 11-3 | 967 | 0.1 | 5 | 7.5 | 978 | 11 |

### Example 4: Reductive Amination Chemistry (RAC) Conjugation for Azidopropylamine Incorporation

The overall process for TEMPOX oxidation/RAC involves (i) conversion of primary alcohols to carbonyls on Pn3 polysaccharide, (ii) incorporation of 3-azidopropylamine into Pn3 polysaccharide via imine bonds and finally (iii) reduction of the imine bond using sodium cyanoborohydride (NaBH3CN) to a secondary amine bond using RAC.

The TEMPOX oxidized polysaccharide (Sample IDs 11-2 and 11-3) were pooled to proceed to RAC conjugation with azidopropylamine. Briefly, Pn3_{TEMPOX} was mixed with buffer and adjusted with WFI to a final concentration of 5 mg/ml polysaccharide in 0.1M phosphate pH 6.5. 3-azidopropylamine in dimethyl sulfoxide (DMSO) was added to the reaction at a molar ratio of 10 Meq 3-azidopropylamine:aldehyde. The reaction temperature was increased to 37° C. and aged sodium cyanoborohydride (NaBH₃CN) was added at a ratio of 1.2 Meq NaBH₃CN:Pn3. The conjugation proceeded for 48±4 hrs. In some experiments, sodium borohydride (NaBH₄) was added at 2 Meq NaBH₄:Pn3 for 3 hrs at the end of conjugation prior to purification to reduce (cap) any unreacted aldehydes on the polysaccharide backbone to hydroxyl groups. Purification via UFDF using 50 kDa MWCO membranes and 20× diavolumes against 100 mM phosphate buffer, pH 7 was performed to purify the azide functionalized polysaccharide. SEC-MALLs Mw and percent azide incorporation results are shown in Table 6.

TABLE 6
Characterization of TEMPOX Derived
Azido Serotype Pn3 Polysaccharide

| Sample ID | Azido Poly MW (kDa) | Percent Azide (%) |
|---|---|---|
| 23-4 | 610 | 3.5 |

### Example 5: Click Conjugation of Azido-Pn3 and Alkyne-CRM₁₉₇

The azido incorporated Pn3 polysaccharide intermediates (generated via TEMPOX, CDAP, or DMTMM chemistry) and alkyne-CRM₁₉₇ underwent [3+2] click cycloaddition when catalyzed with Cu(I). Alkynated CRM₁₉₇ was added to the reaction slowly over time using a syringe pump while mixing in a 100 mL automated laboratory reactor system under N2 to limit the undesirable alkyne-alkyne homocoupling and polysaccharide depolymeration by radical oxygen species. Generalized conditions for the click conjugation reaction are shown in Table 7.

TABLE 7
Conditions for Optimum Click Conjugation

| | | Reaction | |
|---|---|---|---|
| Reactants | Oxygen Control | conditions | Purification |
| Azide:Alkyne Molar | Headspace swept | Temp: 35° C. | pH adjustment to |
| Ratio: 1.1:1 | with N₂ gas | pH: 7 | 7.8, UFDF 100 kDa |
| [Aminoguanidine](mM): 5 | phosphate N₂ | CRM₁₉₇- | MWCO |
| [Sodium | degassing of | PPGₙ: dosing | Ultracellulose or |
| Abscorbate](mM): 5 | sodium | over 16 hrs of | PES Biomax |
| [THPTA]:[CuSO₄]: 5:1 | abscorbate for 30 | 18 hr reaction | membrane with 10 |
| CRM₁₉₇-PPGₙ: 3-9 | min prior to | Quench with | mM phosphate pH 7 |
| [EDTA]: 45 mM | addition | EDTA and | |
| | | hold for 1 hr | |
| | | under N₂ | |

### Example 6: PADRE and Bromelain Derivatized CRM₁₉₇

CD4+ T cells play a central role in orchestrating immunity and in priming and maintaining CD8+ T cell effector functions [Ghaffari-Nazari et al. (2015) PLoS One 10(11):e0142563].

Immune responses have been enhanced by including CD4+ T cell epitopes in peptides vaccines [Muranski and Restifo (2009) Curr Opin Immunol 21(2):200-208; Lai et al. (2011) ISRN Immunology ID:497397]. Pan DR-binding epitope (PADRE) is a universal synthetic 13-15 amino acid peptide that activates CD4+ T cells. This synthetic peptide was designed to bind most of the human HLA-DR receptors, providing “universal” immune stimulation in heterogeneous populations [Alexander et al. (1994) Immunity 1:751-761]. The presence of a universal T helper epitope such as PADRE greatly improved antibody immune responses in variety of preclinical vaccines such as the malaria recombinant antigen vaccine [Rosa et al. (2004) Immunology letters 92(3):259-268], the dengue peptide vaccine [Chan et al. (2020) Vaccines 8(417):18], and the Alzheimer's vaccine [Agadjanyan et al. (2005) J Immunology 174(3):1580-1586].

The PADRE peptides used in our studies were given the names PADRE 1, PADRE 2, and PADRE 3. The sequences of each PADRE peptide are described in Table 8. PADRE 1 utilized two unnatural amino acids, cyclohexylalanine {Ahx} and aminocaproic acid {CHA}. All the PADRE peptides possess D-alanine at the N-terminus. Substitution of D-alanine residues may minimize exopeptidase degradation of PADRE peptides in APCs [Tugyi et al. (2005) PNAS 102(2):413-8]. PADRE 1 and 3 are novel versions of PADRE synthesized (“tagged”) to possess a propargyl glycine {Pra} on the C-terminus of the peptide to enable use in click conjugation to a specific site on the peptide. PADRE 1 and 3 were tagged for click conjugation to Pn3-azido activated polysaccharide, and PADRE 2 was tagged for direct conjugation using either CDAP or DMTMM chemistry.

TABLE 8
PADRE sequences utilized in direct conjugation and click
conjugation experiments

| Name | Sequence | Length |
|---|---|---|
| PADRE 1 | {d-ALA}K{CHA}VAAWTLKAA{d-ALA}{Ahx}{Pra} | 15 |
| PADRE 2 | {d-ALA}KVAAWTLKAA{d-ALA} | 13 |
| PADRE 3 | {d-ALA}KVAAWTLKAA{d-ALA}{Pra} | 14 |

Cross-reactive material 197 (CRM₁₉₇) contains T cell helper epitopes that play a critical role in Prevnar 13© and Prevnar20™ immune response. CRM₁₉₇ is a nontoxic mutant with a single amino acid substitution that inactivates the enzyme [Malito et al. (2012) PNAS 109(14):5229-5234]. From the 40 possible primary amines on CRM₁₉₇, only a few (K103, K498, and K526) participated in p-nitrophenol (PNP) and di-succinimido-adipate (DSA) synthesized conjugates [Moginger, U., Resemann, A., Martin, C. et al. Sci Rep 6, 20488 (2016):1-13]. Only surface accessible lysines exhibited moderate to high conjugation frequency. Based on amino acid databases, additional T-cell epitopes may exist within the hydrophobic core of CRM₁₉₇ but lysine accessibility within the core structure of the CRM₁₉₇ may limit the utilization of these conjugation sites. To access these underutilized conjugation sites, CRM₁₉₇ was cleaved into a heterogenous size distribution of peptides via proteolysis. These CRM₁₉₇ peptides, carrying once inaccessible lysine conjugation sites, were utilized in click conjugation to serotype 3 polysaccharide.

Cathepsins found in APCs are enzymes involved in antigen processing. Cathepsins include four classes of enzymes with unique substrate specificity: cysteine endopeptidases (Cathepsins B, C, F, H, K, L, O, S, V, W, and X), aspartyl cathespsins (D,E), serine cathepsins (A, G), and asparagine endopeptidase. Cathepsin S is an elastase and is critical to the final step of invariant (Ii) chain processing to form a class-II-associated invariant chain (CLIP) prior to antigen presentation in endosomal APCs [S. van Kasteren (2014) Curr Opin Chem Biol 23:8-15]. Cathepsin S cleaves hydrophobic Ii chain intermediates at the P2 position (valine, methionine and norleucine) and P1′ (between Gly and Leu residues) positions [Turk et al. (2012) Biochimica et Biophysica Acta 1824(1):68-88]. Within the Cathepsin S class of proteases lies commercially available and affordable protease candidates, papain (papaya tree) and bromelain (pineapple). Bromelain and papain both possess cleavage sites (Glu-Phe-Leu) that are more specific than the observed cleavage site sequences found in proteases such as trypsin and thus may preserve MHCII epitope sequences within CRM₁₉₇. An additional benefit of these proteases involve their plant based origin which eliminates potential concerns usually associated with intermediates from animal-derived raw materials. Based on this reasoning, CRM₁₉₇ was derivatized with bromelain (stem), the resulting peptides were purified, alkynated, and the activated CRM₁₉₇ peptides were click conjugated to azide incorporated Pn3 polysaccharides (CDAP and DMTMM derived).

### Example 7: PADRE Gellinq Events and Solubility

CDAP and DMTMM direct incorporation of PADRE 2 peptide resulted in significant gelling of the PADRE 2 and the Pn3 polysaccharide (unreported studies). The DMSO based reaction with Pn3 polysaccharide results in a gelation event. It has been demonstrated that ionic gelation is possible using polysaccharide and PADRE peptides in the formation of nanoparticles [Correia-Pinto et al. (2015) Vaccines 3(3):730-750]. The ionic gelation kinetics may outpace the CDAP or DMTMM kinetics based on the ionic environment in the reaction. The treatment of alkynated PADRE with azide incorporated Pn3 polysaccharide disclosed herein eliminates the need for the direct incorporation of PADRE using either CDAP or DMTMM in addition to the low ionic strength buffer used in the current click conjugation strategy disclosed herein.

It is possible that the PADRE constructs disclosed herein may have solubility limitations in aqueous solutions (<5 mg/ml max concentration) versus DMSO solutions (<10 mg/ml max concentration), so conditions for PADRE solubility were adjusted to achieve optimum click conjugation efficiency. Ionic gelling may have occurred during the CDAP or DMTMM direct incorporation of PADRE 2, as buffer matrix with high ionic strength in higher concentrated Pn3 solutions promotes gelling. Aggregation with an alkynated CRM₁₉₇ batch fed approach was also observed in click conjugation reactions. To avoid potential solubility issues, the following conditions with PADRE 3 were proposed: 1. Lower the ionic strength of the reaction matrix and diafiltration matrix to 10 mM phosphate pH 7.0, 2. Slow dose addition of alkynated PADRE 3 into the reaction, and 3. make stock solution of alkynated PADRE 3 in water instead of DMSO to eliminate the need for a lyophilization step. As a result of these efforts, the PADRE stock did not come out of solution after 18 hr at room temperature. No gelling or aggregation events were observed with the PADRE 3 during click conjugation.

### Example 8: Limiting Alkyne-Alkyne Homocouplinq

Early click conjugation work exhibited Pn3 click conjugates that were large in MW and contained high unconjugated polysaccharide. SEC-MALLs results confirmed that the resulting conjugates carried significantly more conjugated protein than conjugated polysaccharide. It has been determined that the potential for the Glaser coupling between CRM₁₉₇-PPGₙ occurs due to an increased concentration of alkyne in the presence of 02 and copper during the click conjugation reaction [A. Hay (1962) J Org Chem 27(9):3320-3321; Bock et al. (2006) Eur J Org Chem 1:51-68]. The Glaser coupling side reaction may outcompete the desired click reaction for alkynated species of larger molecular size due to steric hindrance and/or diffusion limitations and so Glaser coupling may also be minimized by use of smaller alkynated peptides.

To limit the effect of Glaser coupling, CRM₁₉₇-PPGₙ was dosed at a slow addition rate over 16 hr of the 18 hr click conjugation reaction to lower the concentration of unreacted CRM₁₉₇-PPGₙ available during the click conjugation. Application of this reaction strategy to slowly add CRM₁₉₇-PPGₙ resulted in click conjugates that not only met the molecular weight target of >1000 kDa, but also met the S:P (0.5-2.5) and free saccharide % (<30%) targets.

### Example 9: Incorporation of 3-azidopropylamine Controlled by Pn3 Polysaccharide:DMTMM Ratio

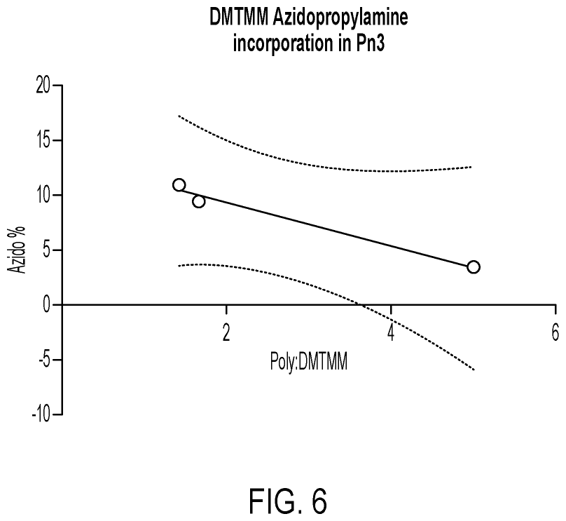

DMTMM incorporates 3-azidopropylamine into Pn3 polysaccharide proportional to increasing DMTMM concentration. To determine the optimum conditions for 3-azidopropylamine incorporation, the Pn3 polysaccharide to DMTMM ratio by mass was varied from 0.143, 0.167, and 5 (FIG. 6). The number of carboxylic groups in Pn3 serving as triazine activation sites were in excess for this reaction. Higher concentrations of DMTMM allow for higher degrees of substitution as long as sufficient triazine activation sites are available [S. Rydergren (2013) Doctoral Thesis, Uppsala University]. The use of 3-azidopropylamine as the amine bypasses the precipitation observed in conjugation reactions with DMTMM at high concentrations with bulkier and more hydrophobic amine constituents, although it was a challenge to find the appropriate Pn3 polysaccharide:DMTMM ratio for the desired 3-azidopropylamine incorporation. Lyophilization was utilized to concentrate Pn3 polysaccharide prior to DMTMM activation.

DMTMM facilitates an efficient one-step condensation of biopolymers over a wide pH range in comparison to the pH dependent EDC/NHS chemistry. EDC/NHS coupling requires accurate pH control. The generation of the NHS ester at the carboxylic acid group of the polymer target requires an acidic pH, while the nucleophilic attack of the amine is only efficient at neutral or alkaline pH where the NHS ester is hydrolytically liable [Schante et al. (2011) Carbohydrate 85:469-489]. Therefore EDC/NHS driven amidation favors slightly acidic environments [E. Sturabotti (2020) Doctoral Thesis, Department of Chemistry, Sapienza University, Rome]. Use of DMTMM for bioconjugations have been reported at both alkaline [Pelet and Putnam (2011) Bioconjugate Chem 22:329-337] and acidic pH [Nimmo et al. (2011) Biomacromolecules 12:824-830; Yu et al. (2014) Polymer Chemistry 5:1082-1090], though polysaccharides treated with DMTMM at mildly acidic pH have exhibited increased crosslinking.

DMTMM provides a simple green chemistry alternative for incorporating functional amine into polysaccharides. The primary by-product of EDC/NHS amidation is a N-acylurea from the electrostatic displacement of the o-acylisurea intermediate. N-acylurea has demonstrated insecticidal and anti-tumoral properties. Proper removal and elimination of this side product is a vital and cost saving aspect in the formulation of drug product. DMTMM amidation replaces potentially harmful side products observed in EDC/NHS chemistry in lieu of an insoluble hydroxytriazine by-product which can be easily removed by filtration or other common purification techniques.

### Example 10: Synthesis of VOB Click Conjugates for Murine Immunogenicity Studies

Novel Streptococcus pneumoniae serotype 3 (Pn3) conjugates were generated. Pn3 was conjugated to CRM₁₉₇ carrier protein using different Click chemistries to target the conjugation of Pn3 polysaccharide to the carrier protein from either polysaccharide carboxylate, primary alcohol, or hydroxyl functional groups as described in Table 9. These Pn3 conjugates were evaluated for impact on murine immunogenicity compared to a Pn3-CRM₁₉₇ conjugate generated using standard reductive amination chemistry in aqueous buffer (RAC/aqueous) with a CRM₁₉₇ carrier protein, referred to here as “RAC/aqueous process” (see e.g. example 1 of Application Number: PCT/IB2022/054914).

Table 9 also details select experimental parameters and results for the click conjugates included in the murine immunogenicity studies. Samples with similar azide inputs (3 and 3.5% vs 6.5 and 9.5%) demonstrate a similar SPR and free saccharide %. The Pn3_{TEMPOxRAC} (“Click A”) conjugate exhibited the lowest conjugate size, % saccharide <0.3 K_{d} and conjugate molecular weight compared to Pn3_{CDAP} (“Click B”) and Pn3_{DMTMM} (“Click C” and “Click D”) conjugates. The lower conjugate molecular weight Pn3_{TEMPOxRAC} could stem from limited number of azides on the Pn3 polysaccharide, the location of the pendant azides and their limited accessibility during click conjugation. Pn3_{CDAP} and Pn3_{DMTMM} displays a full extent of reaction with higher azide inputs (6.5 and 9% respectively). The azide concentration was the rate limiting component for the click conjugation reaction with alkynated CRM₁₉₇ as the excess reagent. Increasing alkyne content greater than 7.88 mol alkyne/mol CRM₁₉₇ promotes alkyne-alkyne homocoupling. Regardless of scale or chemistry, low free saccharide (<30%) of the conjugate, high molecular size, %</=0.3 K_{d} (>40%) and molecular weight (>1000 kDa) was achieved for all click conjugates except for Pn3_{TEMPOxRAC}.

TABLE 9
Assay results for Serotype 3 click conjugates used in murine studies

| | RAC/aqueous | | | | |
|---|---|---|---|---|---|
| | Process | Click A | Click B | Click C | Click D |
| Pn3 Azido | RAC/Aqueous | TEMPOx/ | CDAP | DMTMM | DMTMM |
| Derivitization | | RAC/Aqueous | | | |
| Chemistry | | | | | |
| Azide Target | Glc C2, C3 | Glc C6 | OH | GlcA C6 | GlcA C6 |
| Location in | | | (non-specific) | | |
| Pn3 Structure | | | | | |
| Pn3 Azide | N/A | 3 | 6.5 | 3.5 | 9.5 |
| Content | | | | | |
| (mol %/PSRU) | | | | | |
| Conjugate | 2467 | 686 | 1224 | 1767 | 1855 |
| MW (kDa) | | | | | |
| SPR Ratio | 0.90 | 1.65 | 0.64 | 1.30 | 0.71 |
| Free | <5 | 25 | 17 | 29 | 17 |
| Saccharide, % | | | | | |
| Saccharide | 0.55 | 1.05 | 0.73 | 1.06 | 1.12 |
| Anthrone | | | | | |
| (mg/mL) | | | | | |
| Protein Lowry | 0.60 | 0.64 | 1.14 | 0.80 | 1.70 |
| (mg/mL) | | | | | |

MW: molecular weight;
SPR: Saccharide to protein ratio

Female, Swiss Webster mice (6-8 weeks of age,) were immunized two times subcutaneously (250 μL) at weeks 0 and 3 with Pn3 glycoconjugates to evaluate immunogenicity. Glycoconjugates were administered at 0.01 or 0.1 μg/dose with 100 μg/dose of AIPO₄ as an adjuvant. Vaccine doses were calculated based on anthrone saccharide concentration for each conjugate. All preclinical immunogenicity studies were powered to detect a 4 to 5-fold difference in opsonophagocytic (OPA) titers using 25 mice per group. Whole blood was collected from mice two weeks after the second vaccination (Week 5, PD 2) and sera used for OPA analyses. OPA data is presented as titer and % mice that did not reach a titer above limit of detection (LOD, % non- responders). Significant differences (P<0.05) in immunogenicity (increased fold GMT) as determined by robust linear regression and decrease in % responders were used as criteria for subsequent evaluation of candidates.

OPA assays have been previously described (Gray 1990, Romero-Steiner, Libutti et al. 1997, Hu, Yu et al. 2005, Gooper, Yu et al. 2011). The OPA assay for S. pneumoniae serotype 3 is briefly described as follows: Heat-inactivated sera were serially diluted 2.5-fold in Hank's balanced saline solution supplemented with 0.1% gelatin. Target bacteria were added to assay plates and were incubated for 30 min at 25° C. on a shaker. Baby rabbit complement (3- to 4-week old, Pel-Freez, 12% final concentration) and differentiated HL-60 cells were then added to each well at an approximate effector to target ratio of 200:1. Assay plates were incubated for 45 minutes at 37° C. with shaking. 10-μL aliquot was transferred to the wells of Millipore, MultiScreenHTS HV filter plates containing 50 μL of 2% HL-60 lysate in distilled water. Liquid was filtered through the plates under vacuum, and 50 μL of HySoy medium supplemented with Kellogs solution was added to each well and filtered through. The filter plates were then incubated at 37° C., 5% C02 overnight and the resulting colonies were then stained with Coomassie Brilliant Blue stain. Colonies were imaged and enumerated on a Cellular Technology Limited (CTL) ImmunoSpot Analyzer®. The interpolated OPA antibody titer is the reciprocal of dilution that yields a 50% reduction in the number of bacterial colonies when compared to the control wells that did not contain immune serum.

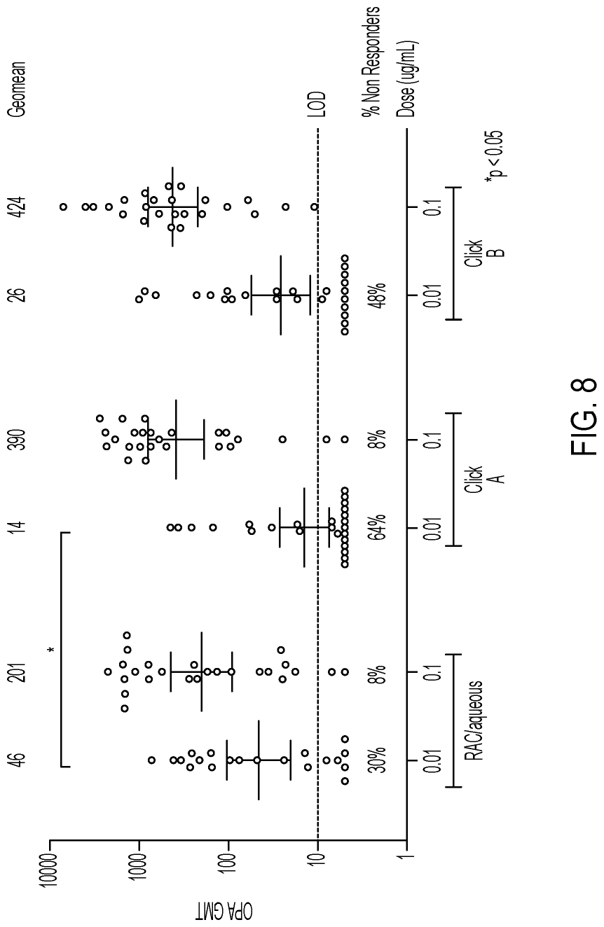

The first study compared the RAC/aqueous Pn3 with two Click conjugates (Click A and B; see FIG. 8) which were generated by altering the conjugation process by which the CRM₁₉₇ carrier protein was conjugated to the Pn3 polysaccharide. Conjugation targeted either the primary alcohol of glucose (Click A) or non-specific hydroxyl group activation (Click B). Overall, the responses were dose dependent and resulted in an increased geometric mean titer as measured by the OPA assay and decreased % non-responders with higher conjugate dose. At the 0.01 μg dose level, the RAC/aqueous process Pn3 elicited a higher OPA response and lower % non-responders compared to the Click A and Click B conjugates. The difference in OPA was significantly higher (P=0.0215) between the RAC/aqueous and Click A. At the 0.1 μg dose, Click A and Click B elicited a higher OPA response compared to the RAC/aqueous process, although these differences were not statistically significant.

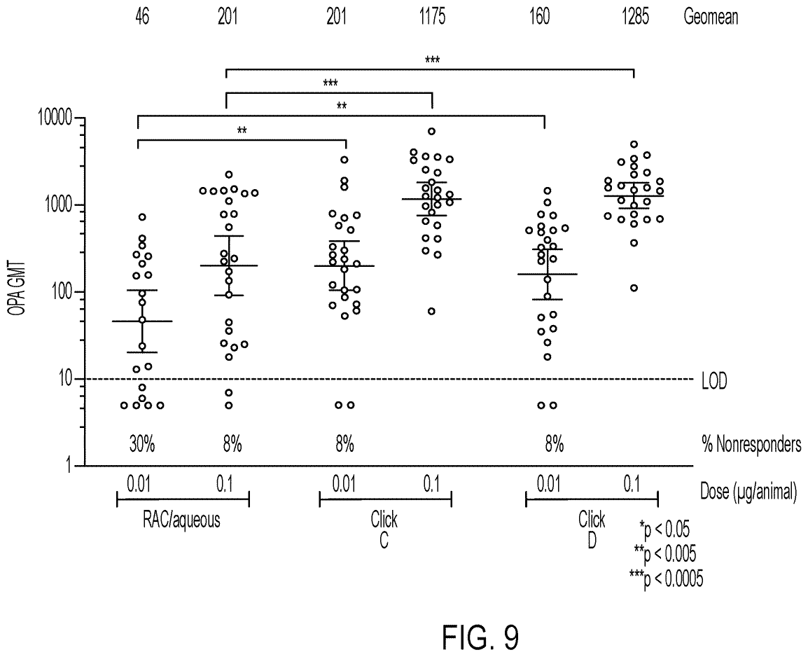

A second study was run to compare the RAC/aqueous process Pn3 to two Click conjugates (Click C and D; see FIG. 9) that conjugate Pn3 polysaccharide to CRM₁₉₇ carrier protein at the carboxylate group of glucuronic acid at a molar azide incorporation content of 3.5% or 9.5% on a molar basis per Pn3 repeat unit, respectively. Similar to the results displayed in FIG. 8, the responses were dose dependent and resulted in an increased geomean and decreased % non-responders with higher conjugate dose. At the 0.1 μg dose, Click C and Click D both elicited a significantly higher antibody response than the RAC/aqueous process (P=0.0011 and P=0.0055, respectively). The % non-responders was also lower with Click C and Click D compared to the RAC/aqueous process. Results were similar at the 0.01 μg dose with Click C and D eliciting a significantly higher GMT than the RAC/aqueous process and lower % non-responders (P=0.0001 and P<0.0001, respectively).

In summary, the Pn3-CRM₁₉₇ click conjugates targeting Pn3 polysaccharide conjugation specifically to the primary hydroxyl group at glucose carbon 6 (Click A) elicited a statistically lower immune response in mice compared to Pn3 RAC/aqueous process glycoconjugate at 0.01 μg dose. Pn3-CRM₁₉₇ click conjugates targeting Pn3 non-specific conjugation to hydroxyl groups (Click B) elicited a comparable immune response in mice compared to the Pn3 RAC/aqueous process glycoconjugate at 0.01 and 0.1 μg doses. In contrast, Pn3-CRM₁₉₇ Click conjugates targeting Pn3 conjugation specifically to the carboxylate group at glucuronic acid carbon 6 (Click C and Click D) resulted in an improved immune response in mice compared with RAC/aqueous process serotype 3. Vaccination with improved Click conjugates (Click C and Click D) elicited a statistically higher OPA response and a lower % non-responder at two vaccination doses compared to RAC/aqueous process Pn3.

This data demonstrates that immunogenicity of S. pneumoniae vaccine conjugates, such as Pn3 may be improved by changing the process used for conjugation.

### Example 11. Bromelain Derivatization of CRM₁₉₇ and Bromelain Derivatized CRM₁₉₇ Alkynation with PPG-NHS Ester

Significant development work was conducted to identify the proper units of bromelain activity to add to the CRM₁₉₇ digests to achieve a heterogenous size distribution of CRM₁₉₇ peptides. The formulated bromelain powder used in these studies possessed 5.0 Units of hydrolytic activity per mg of enzyme. For these digests, a working stock solution of bromelain of 100 mg/ml was prepared. A reaction concentration of 10 mg/ml of bromelain was incubated with varying CRM₁₉₇ reaction concentrations (0.88 to 3.53 mg/ml) at 37° C. in pH 4.5. The reactions proceeded for 2 hrs with samples pulled every 30 minutes for protein concentration by Lowry, and purity by both SDS-PAGE and BEH300-HPLC analysis. The hydrolyzed CRM₁₉₇ peptides were diafiltered (5 diavolumes with phosphate buffered saline) using a the 5 KDa MWCO cellulose filter. The 5 k CRM₁₉₇ peptide permeate was concentrated (2 mg/ml to 4 mg/ml) and diafiltered (5 diavolumes with phosphate buffered saline) with a 1 KDa MWCO cellulose filter step at a membrane challenge of 20 gram/m². For post 5K filtration, the Lowry result for all four samples ranged between 0.5-1.42 mg/ml. Based on the Lowry results of the 5 k samples, the 1 K filtration step was conducted and obtained Lowry results for the 1K step ranging from 0.05 mg/ml, 0.14 mg/ml, and 1.45 mg/ml. The 1K filtrate underwent HPLC analysis using a XBridge BEH300 C18 column set at 50° C. A filtrate sample contained CRM₁₉₇ peptides with molecular weights with an average molecular weight of 24 kDa. The higher concentrated peptide filtrate (1.45 mg/ml concentration) were utilized for alkynation. The derivatized CRM₁₉₇ peptides were alkynated in the presence of PPG-NHS ester. The target peptide concentration of 2 mg/ml and 20 Meq of PPG-NHS were added to the reaction. The reaction was agitated at 200 rpm while the temperature was held at 23° C. overnight. The alkynated CRM₁₉₇ peptides were diafiltered over a 0.1 m² 1 KDa MWCO filtration step with 10 diavolumes of phosphate buffered saline and at a 2 mg/ml retentate concentration. The recovered retentate fed subsequent DMTMM and CDAP click conjugate reactions.

### Example 12. PADRE and Bromelain Derivatized CRM₁₉₇ Peptide Click Conjugation into CDAP and DMTMM Activated Pn3 Polysaccharide

A reaction was run omitting DMSO from a click conjugate protocol for alkynated trimeric gp120 Epitope mimic peptides using 5 mM Sodium ascorbate, 5 mM aminoguanidine, 0.1 mM CuSO4, and 0.5 mM THPTA ligand in 10 mM phosphate buffer at pH 7.4 containing 5% DMSO for 18 hr [Schellinger et al. (2011) J Am Chem Soc 133(10):3230-3233]. The PADRE peptide solution stayed soluble at 37° C. in the incubator at 200 rpm agitation. For the CDAP Pn3-PADRE 3 click conjugation, azide to alkyne ratio 0.5-1.0 (mol/mol) was targeted using a Pn3 polysaccharide with a 13% incorporated azide and PADRE with 1 mol alkyne per mol peptide. For the DMTMM Pn3-PADRE 3 click conjugation, azide to alkyne ratio 0.5 (mol/mol) was targeted using a Pn3 polysaccharide with a 11-13% incorporated azide and PADRE with 1 mol alkyne per mol peptide. The PADRE peptide was used to prime the dosing pump with a N2 overlay at room temperature. For the CDAP/DMTMM Pn3-CRM₁₉₇ peptide click conjugation, azide to alkyne ratio 0.5 (mol/mol) was targeted using a Pn3 polysaccharide with a 11-13% incorporated azide and PADRE with 1 mol alkyne per mol peptide. The dosing was allowed to continue from 2-16 hrs at 37-55° C. followed by a 2 hr hold. EDTA was added to cease the reaction. A 5 ml sample of the crude conjugate was removed prior to diafiltration. For the diafiltration, we used 100 kDa MWCO regenerated cellulose 88 cm² ultrafiltration cassettes with a membrane challenge of 5.4 g/m². We targeted a retentate concentration of 1 mg/ml with an expected yield across the TFF step of 75%.

TABLE 10
Assay results for Serotype 3 PADRE 3 peptide click conjugates

| | Conjugate # | | | |
|---|---|---|---|---|
| | 1 | 2 | 3 | 4 |
| | Conjugation Reaction Conditions | | | |
| | CDAP 37° C. | CDAP 37° C. | | |
| | 18 hr, 500 mM | 18 hr, 5 mM | | |
| | sodium | sodium | DMTMM 55° C. | CDAP 55° C. |
| | abscorbate | abscorbate | 2 hr | 2 hr |
| Pn3-Azide % (mol/mol PSRU) | 13% | 13% | 11% | 13% |
| Pn3-Azide MW (kDa) | 3586 | 3586 | 545 | 3586 |
| Conjugate MW (kDa) | 325 | 582 | 290 | 693 |
| Conjugate Saccharide:Protein | 0.83 | 0.96 | 0.56 | 0.65 |
| Ratio, w/w (SPR) | | | | |
| Conjugate Free Saccharide | 38% | 29% | 16% | 18% |
| (%), DOC-HCl method | | | | |
| Conjugate Molecular Size % | 35% | 54% | 27% | 65% |
| Saccharide </= 0.3 Kd | | | | |
| Conjugate Endotoxin (EU/μg | N/D | N/D | N/D | 0.05 |
| poly repeat unit) | | | | |

Table 10 details select experimental parameters and results for click conjugates Pn3_{PADRE3-CDAP}, Pn3_{PADRE3-DMTMM} (ranging from 11-13% azide) in a 60 ml reaction volume. Comparing Pn3_{PADRE3-CDAP} and Pn3_{PADRE3-DMTMM} conjugates, Pn3_{PADRE3-CDAP} conjugates demonstrate a higher conjugate molecular weight. Pn3_{PADRE3-CDAP} conjugates also exhibit limited extent of reaction when incubated at 18 hr resulting in higher SPR, higher free saccharide, and lower K_{d}. The SPR and free saccharide decreased while K_{d} increased as the reaction temperature was increased to 55° C. and reaction time reduced to 2 hr. The extent of reaction increased as the increase in reaction temperature enhanced the click reaction rate. The Pn3_{PADRE3-DMTMM} conjugation demonstrated a conjugate characteristic (higher molecular weight, lower SPR, and lower free saccharide) similar to the Pn3_{PADRE3-CDAP} conjugate produced at an increased reaction temperature and reduced reaction times. The reduced size of the carrier (PADRE peptide vs CRM₁₉₇ protein) allowed for higher reaction temperatures to be used during the click conjugation reaction without fear of heat induced denaturation of the carrier protein. Lower than expected peptide conjugate molecular weights were observed with starting polysaccharide molecular weights decreasing by 50% or more. This may have been a result of oxidative depolymerization of polysaccharide occurring during the click conjugation reaction.

TABLE 11
Assay results for Serotype 3 CRM₁₉₇ click conjugates

| | Conjugate # | |
|---|---|---|
| | 1 | 2 |
| | Conjugation Reaction Conditions | |
| | DMTMM 55° C. | CDAP 55° C. |
| | 2 hr | 2 hr |
| Pn3-Azide % (mol/mol PSRU) | 11 | 13 |
| Pn3-Azide MW (kDa) | 545 | 1335 |
| Conjugate MW (kDa) | 966 | 1224 |
| Conjugate Saccharide:Protein Ratio, | 1.06 | 0.64 |
| w/w (SPR) | | |
| Conjugate Free Saccharide (%), | 16.8 | 17.3 |
| DOC-HCl method | | |
| Conjugate Molecular Size % | 45 | 40 |
| Saccharide </= 0.3 Kd | | |
| Conjugate Endotoxin (EU/μg poly | N/D | N/D |
| repeat unit) | | |

Table 11 details select experimental parameters and results for click peptide conjugates Pn3_{CRMPep-CDAP}, Pn3_{CRMPep-DMTMM} (ranging from 11-13% azide) in a 60 ml reaction volume. Preferred conditions from the PADRE peptide conjugates click reactions (high temperature and short reaction times) were utilized to create CRM₁₉₇ peptide conjugates. Comparing PADRE peptide and CRM₁₉₇ peptide conjugates, CRM₁₉₇ peptide conjugate demonstrates a larger conjugate molecular weight with little to no drop in starting polysaccharide molecular weight. The free saccharide and K_{d} between both conjugates made in similar conditions were virtually identical. In the case of CRM₁₉₇ peptide conjugates made from DMTMM and CDAP derived Pn3 polysaccharides, the SPR is higher for the DMTMM CRM₁₉₇ peptide conjugate compared to the CDAP CRM₁₉₇ peptide conjugate. The lower SPR may indicate a higher degree of conjugation in the case of the CDAP CRM₁₉₇ peptide conjugate. The DMTMM CRM₁₉₇ peptide conjugate exhibited higher than expected K_{d} compared to the DMTMM PADRE peptide conjugate. Alternatively, the CDAP CRM₁₉₇ peptide conjugate exhibited lower than expected K_{d} compared to the CDAP PADRE peptide conjugate.

### Example 13. Effect of Nitrogen Sparqed and Sodium Ascorbate Dosed Solutions on Oxidative Depolymerization in Click Conjugations

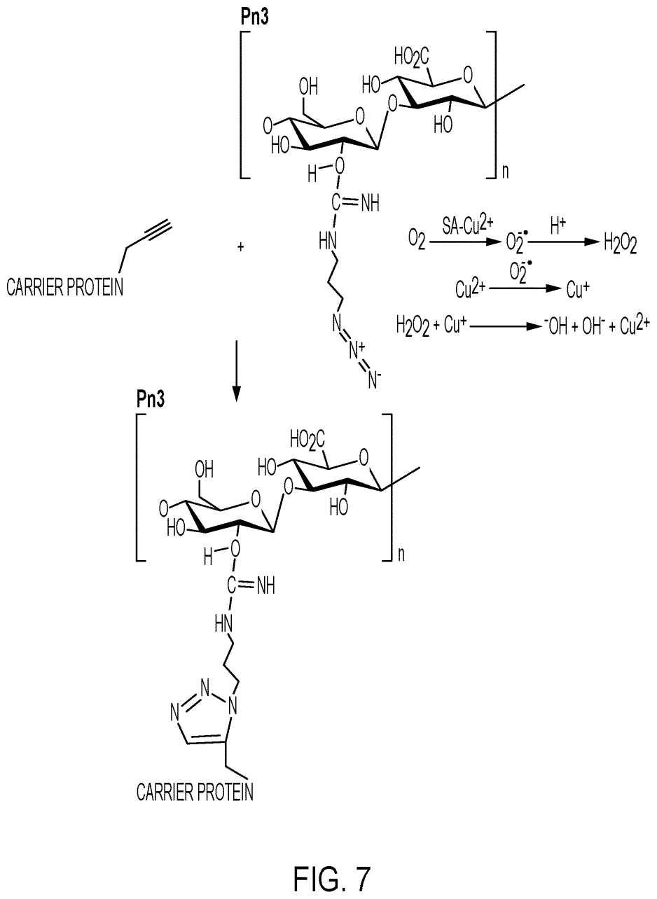

Oxygen radical generating systems (such as ascorbic acid-copper ion) have an effect on the oxidative depolymerization of several polysaccharides [Uchida and Kawakishi (1986) Agricultural and Biological Chemistry 50(10):2579-2583]. The ascorbic acid-copper ion system converts diatomic oxygen to subsequent oxygen free radicals (FIG. 7). The oxygen free radicals combine with diatomic hydrogens to form the hydrogen peroxide species. The copper (II) is simultaneously reduced to copper (I) for use in the click conjugation. The resulting copper (I) species is consumed in a side reaction that competes with the click conjugation reaction. Copper (I) reacts with the formed hydrogen peroxide species to create hydroxide free radical species which may result in the depolymerization of the polysaccharide. In previous attempts of forming the PADRE peptide click conjugate, unexplained drops in polysaccharide size and conjugate K_{d} were observed.

This drop in molecular weight and K_{d} may be a byproduct of the ascorbic acid-copper radical generating system. The conditions used to test this hypothesis were the following: 500 mM sodium ascorbate and a lower concentration of 5 mM sodium ascorbate. In Table 10, the molecular weight for the PADRE peptide conjugate produced using 5 mM sodium ascorbate is higher (582 kDa) compared to PADRE peptide conjugate using 500 mM sodium ascorbate (325 kDa). This evidence confirms free radical depolymerization of the polysaccharide as a source of the reduction in conjugate molecular weight and reinforces the value of controlling oxygen levels during click bioconjugation reactions.

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. A method of making a capsular saccharide glycoconjugate, comprising the steps of:
(a) contacting an isolated Streptococcus pneumoniae capsular saccharide with an azido linker and 4-(4,6-dimethoxy [1,3,5]triazin-2-yl)-4-methyl-morpholinium (DMTMM) to produce an azido incorporated saccharide;
(b) contacting a carrier protein with an agent bearing an N-hydroxysuccinimide (NHS) moiety and an alkyne group to produce an alkyne functionalized carrier protein; and
(c) reacting the azido incorporated saccharide with the alkyne functionalized carrier protein by Cu⁺¹ mediated azide-alkyne cycloaddition reaction,
thereby forming the capsular saccharide glycoconjugate.

2. A method of making a capsular saccharide glycoconjugate, comprising the steps of:
(a) contacting an isolated Streptococcus pneumoniae capsular saccharide with 1-cyano-4-dimethylaminopyridinium tetrafluoroborate (CDAP) to produce a reactive intermediate;
(b) contacting the reactive intermediate with an azido linker to produce an azido incorporated saccharide;
(c) contacting a carrier protein with an agent bearing an N-hydroxysuccinimide (NHS) moiety and an alkyne group to produce an alkyne functionalized carrier protein; and
(d) reacting the azido incorporated saccharide with the alkyne functionalized carrier protein by Cu⁺¹ mediated azide-alkyne cycloaddition reaction,
thereby forming the capsular saccharide glycoconjugate.

3. A method of making a capsular saccharide glycoconjugate, comprising the steps of:
(a) contacting a primary alcohol of an isolated Streptococcus pneumoniae capsular saccharide with 4-Acetamido-2,2,6,6-tetramethyl-1-oxopiperidinium tetrafluoroborate (TEMPOX) to produce a reactive saccharide intermediate;
(b) contacting the reactive saccharide intermediate with an azido linker to form an imine bond and produce an azido saccharide intermediate;
(c) contacting the azido saccharide intermediate with sodium cyanoborohydride (NaBH₃CN) to reduce the imine bond to an amine bond and produce a reduced azido incorporated saccharide;
(d) contacting a carrier protein with an agent bearing an N-hydroxysuccinimide (NHS) moiety and an alkyne group to produce an alkyne functionalized carrier protein; and
(e) reacting the reduced azido incorporated saccharide with the alkyne functionalized carrier protein by Cu⁺¹ mediated azide-alkyne cycloaddition reaction,
thereby forming the capsular saccharide glycoconjugate.

4. The method of any one of claims 1-3, wherein the Streptococcus pneumoniae is selected from the group consisting of Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.

5. The method of any one of claims 1-4, wherein the azido linker is a compound of formula (I): 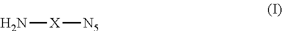
wherein X is selected from the group consisting of CH₂(CH₂)ₙ,
(CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.

6. The method of any one of claims 1-4, wherein the azido linker is a compound of formula (II):

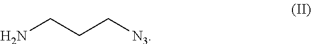

7. The method of any one of claims 1-6, wherein the agent bearing an N-Hydroxysuccinimide (NHS) moiety and an alkyne group is an agent bearing an N-Hydroxysuccinimide (NHS) moiety and a terminal alkyne.

8. The method of any one of claims 1-6, wherein the agent bearing an N-Hydroxysuccinimide (NHS) moiety and an alkyne group is a compound of formula (III): 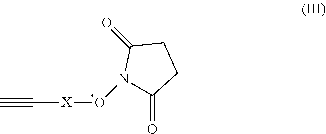
where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

9. The method of any one of claims 1-6, wherein the agent bearing an N-Hydroxysuccinimide (NHS) moiety and an alkyne group is a compound of formula (IV):

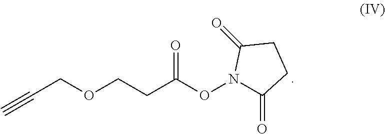 [SMILES: C#CCOCCC(=O)ON1C(=O)CCC1=O]

10. The method of any one of claims 1-9, wherein the carrier protein is selected from the group consisting of CRM₁₉₇, Streptococcus C5a peptidase (SCP), diphtheria toxoid (DT), tetanus toxoid (TT), meningococcal outer membrane protein (OMP) complex, non-typeable Haemophilus influenzae protein D, and pan HLA DR-binding epitope (PADRE).

11. The method of any one of claims 1-10, wherein the capsular saccharide glycoconjugate has a molecular weight of >1000 kDA.

12. The method of any one of claims 1-11, wherein the capsular saccharide glycoconjugate has a free saccharide % of <30%.

13. The method of any one of claims 1-12, wherein the capsular saccharide glycoconjugate has a conjugate saccharide to protein ratio of 0.5-2.5.

14. The method of any one of claims 1-13, wherein the azido linker is present in an amount that is between 0.01-10 molar equivalent to an amount of polysaccharide Repeat Unit of the azido incorporated saccharide.

15. The method of any one of claims 1-14, wherein the agent bearing an N-Hydroxysuccinimide (NHS) moiety and an alkyne group is present in an amount that is between 0.1-10 molar equivalents to an amount of lysines on the carrier protein.

16. The method of any one of claims 1-15, wherein step (c) is carried out in an aqueous buffer in the presence of copper (I) as catalyst.

17. The method of any one of claims 1-16, further comprising purifying the capsular saccharide glycoconjugate after step (c).

18. A capsular saccharide glycoconjugate produced according to any one of the methods of claims 1 to 17.


SEQUENCE LISTING (42 sequences)

SEQ ID NO: 1 - DNA, 21 bp, synthetic construct
  misc_feature 1..21: note = "A class" CpG oligonucleotide
  source 1..21: mol_type = other DNA
ggggacgacgtcgtggggggg

SEQ ID NO: 2 - DNA, 21 bp, synthetic construct
  misc_feature 1..21: note = "A class" CpG oligonucleotide
  source 1..21: mol_type = other DNA
ggggacgacgtcgtggggggg

SEQ ID NO: 3 - DNA, 21 bp, synthetic construct
  misc_feature 1..21: note = "B class" CpG oligonucleotide
  source 1..21: mol_type = other DNA
tcgtcgtttttcggtgctttt

SEQ ID NO: 4 - DNA, 21 bp, synthetic construct
  misc_feature 1..21: note = "B class" CpG oligonucleotide
  source 1..21: mol_type = other DNA
tcgtcgtttttcggtcgtttt

SEQ ID NO: 5 - DNA, 24 bp, synthetic construct
  misc_feature 1..24: note = "B class" CpG oligonucleotide
  source 1..24: mol_type = other DNA
tcgtcgttttgtcgttttgtcgtt

SEQ ID NO: 6 - DNA, 24 bp, synthetic construct
  misc_feature 1..24: note = "B class" CpG oligonucleotide
  source 1..24: mol_type = other DNA
tcgtcgtttcgtcgttttgtcgtt

SEQ ID NO: 7 - DNA, 24 bp, synthetic construct
  misc_feature 1..24: note = "B class" CpG oligonucleotide
  source 1..24: mol_type = other DNA
tcgtcgttttgtcgtttttttcga

SEQ ID NO: 8 - DNA, 21 bp, synthetic construct
  misc_feature 1..21: note = B-Class oligonucleotide
  source 1..21: mol_type = other DNA
tcgtcgtttttcggtgctttt

SEQ ID NO: 9 - DNA, 21 bp, synthetic construct
  misc_feature 1..21: note = B-Class oligonucleotide
  source 1..21: mol_type = other DNA
tcgtcgtttttcggtcgtttt

SEQ ID NO: 10 - DNA, 24 bp, synthetic construct
  misc_feature 1..24: note = B-Class oligonucleotide
  source 1..24: mol_type = other DNA
tcgtcgttttgtcgttttgtcgtt

SEQ ID NO: 11 - DNA, 24 bp, synthetic construct
  misc_feature 1..24: note = B-Class oligonucleotide
  source 1..24: mol_type = other DNA
tcgtcgtttcgtcgttttgtcgtt

SEQ ID NO: 12 - DNA, 24 bp, synthetic construct
  misc_feature 1..24: note = B-Class oligonucleotide
  source 1..24: mol_type = other DNA
tcgtcgttttgtcgtttttttcga

SEQ ID NO: 13 - DNA, 22 bp, synthetic construct
  misc_feature 1..22: note = C class CpG Oligonucleotide
  source 1..22: mol_type = other DNA
tcgcgtcgttcggcgcgcgccg

SEQ ID NO: 14 - DNA, 23 bp, synthetic construct
  misc_feature 1..23: note = C class CpG Oligonucleotide
  source 1..23: mol_type = other DNA
tcgtcgacgttcggcgcgcgccg

SEQ ID NO: 15 - DNA, 21 bp, synthetic construct
  misc_feature 1..21: note = C class CpG Oligonucleotide
  source 1..21: mol_type = other DNA
tcggacgttcggcgcgcgccg

SEQ ID NO: 16 - DNA, 19 bp, synthetic construct
  misc_feature 1..19: note = C class CpG Oligonucleotide
  source 1..19: mol_type = other DNA
tcggacgttcggcgcgccg

SEQ ID NO: 17 - DNA, 20 bp, synthetic construct
  misc_feature 1..20: note = C class CpG Oligonucleotide
  source 1..20: mol_type = other DNA
tcgcgtcgttcggcgcgccg

SEQ ID NO: 18 - DNA, 20 bp, synthetic construct
  misc_feature 1..20: note = C class CpG Oligonucleotide
  source 1..20: mol_type = other DNA
tcgacgttcggcgcgcgccg

SEQ ID NO: 19 - DNA, 18 bp, synthetic construct
  misc_feature 1..18: note = C class CpG Oligonucleotide
  source 1..18: mol_type = other DNA
tcgacgttcggcgcgccg

SEQ ID NO: 20 - DNA, 18 bp, synthetic construct
  misc_feature 1..18: note = C class CpG Oligonucleotide
  source 1..18: mol_type = other DNA
tcgcgtcgttcggcgccg

SEQ ID NO: 21 - DNA, 22 bp, synthetic construct
  misc_feature 1..22: note = C class CpG Oligonucleotide
  source 1..22: mol_type = other DNA
tcgcgacgttcggcgcgcgccg

SEQ ID NO: 22 - DNA, 22 bp, synthetic construct
  misc_feature 1..22: note = C class CpG Oligonucleotide
  source 1..22: mol_type = other DNA
tcgtcgttttcggcgcgcgccg

SEQ ID NO: 23 - DNA, 22 bp, synthetic construct
  misc_feature 1..22: note = C class CpG Oligonucleotide
  source 1..22: mol_type = other DNA
tcgtcgttttcggcggccgccg

SEQ ID NO: 24 - DNA, 24 bp, synthetic construct
  misc_feature 1..24: note = C class CpG Oligonucleotide
  source 1..24: mol_type = other DNA
tcgtcgttttacggcgccgtgccg

SEQ ID NO: 25 - DNA, 23 bp, synthetic construct
  misc_feature 1..23: note = C class CpG Oligonucleotide
  source 1..23: mol_type = other DNA
tcgtcgttttcggcgcgcgccgt

SEQ ID NO: 26 - DNA, 22 bp, synthetic construct
  misc_feature 1..22: note = C-Class oligonucleotides
  source 1..22: mol_type = other DNA
tcgcgtcgttcggcgcgcgccg

SEQ ID NO: 27 - DNA, 23 bp, synthetic construct
  misc_feature 1..23: note = C-Class oligonucleotide
  source 1..23: mol_type = other DNA
tcgtcgacgttcggcgcgcgccg

SEQ ID NO: 28 - DNA, 21 bp, synthetic construct
  misc_feature 1..21: note = C-Class oligonucleotide
  source 1..21: mol_type = other DNA
tcggacgttcggcgcgcgccg

SEQ ID NO: 29 - DNA, 19 bp, synthetic construct
  misc_feature 1..19: note = C-Class oligonucleotide
  source 1..19: mol_type = other DNA
tcggacgttcggcgcgccg

SEQ ID NO: 30 - DNA, 20 bp, synthetic construct
  misc_feature 1..20: note = C-Class oligonucleotide
  source 1..20: mol_type = other DNA
tcgcgtcgttcggcgcgccg

SEQ ID NO: 31 - DNA, 20 bp, synthetic construct
  misc_feature 1..20: note = C-Class oligonucleotide
  source 1..20: mol_type = other DNA
tcgacgttcggcgcgcgccg

SEQ ID NO: 32 - DNA, 18 bp, synthetic construct
  misc_feature 1..18: note = C-Class oligonucleotide
  source 1..18: mol_type = other DNA
tcgacgttcggcgcgccg

SEQ ID NO: 33 - DNA, 18 bp, synthetic construct
  misc_feature 1..18: note = C-Class oligonucleotide
  source 1..18: mol_type = other DNA
tcgcgtcgttcggcgccg

SEQ ID NO: 34 - DNA, 22 bp, synthetic construct
  misc_feature 1..22: note = C-Class oligonucleotide
  source 1..22: mol_type = other DNA
tcgcgacgttcggcgcgcgccg

SEQ ID NO: 35 - DNA, 22 bp, synthetic construct
  misc_feature 1..22: note = C-Class oligonucleotide
  source 1..22: mol_type = other DNA
tcgtcgttttcggcgcgcgccg

SEQ ID NO: 36 - DNA, 22 bp, synthetic construct
  misc_feature 1..22: note = C-Class oligonucleotide
  source 1..22: mol_type = other DNA
tcgtcgttttcggcggccgccg

SEQ ID NO: 37 - DNA, 24 bp, synthetic construct
  misc_feature 1..24: note = C-Class oligonucleotide
  source 1..24: mol_type = other DNA
tcgtcgttttacggcgccgtgccg

SEQ ID NO: 38 - DNA, 23 bp, synthetic construct
  misc_feature 1..23: note = C-Class oligonucleotide
  source 1..23: mol_type = other DNA
tcgtcgttttcggcgcgcgccgt

SEQ ID NO: 39 - DNA, 23 bp, synthetic construct
  misc_feature 1..23: note = P class CpG oligonucleotide
  source 1..23: mol_type = other DNA
tcgtcgacgatcggcgcgcgccg

SEQ ID NO: 40 - DNA, 23 bp, synthetic construct
  misc_feature 1..23: note = P class CpG oligonucleotide
  source 1..23: mol_type = other DNA
tcgtcgacgatcggcgcgcgccg

SEQ ID NO: 41 - protein, 950 aa, synthetic construct
  REGION 1..950: note = Enzymatically inactive fragment of SCP
MAKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

SEQ ID NO: 42 - protein, 949 aa, synthetic construct
  REGION 1..949: note = Enzymatically inactive fragment of SCP
AKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ